SEQUENCE LISTING (202 sequences)

SEQ ID NO: 1 - DNA, 1157 bp, synthetic construct
  source 1..1157: mol_type = other DNA
agcaaaatcctgatgcagtatcaggcccgactggagcagagtgagaagaggctaaggcagcagcaggcagagaaggattcccagatcaagagcatcattggcaggtgaggggcggcctggggagggggttgtgagggagagcctgaggctggagagagcaagtgggcgagctactcctctgactcccatccccaaactcaggagcccaccaggagagcccaccactctcctccccaggaagccacccactcactcatcaccagatggagagaaaccccaacctgcttagtgcattaaatatctctttaccaaaccctgacctctcttctgatagagtagcttcggaagcccttggaaaatgtacctgttcctgtccaaccatcactgcatttgcatttaccctaggccagagctccccactagttattctcaacttaacctgtgatgttcactccaaacctaagcagggctccctagccagagtaggccctgcccttcctgggtggaccctccctctctagccttggaaaggtgttctgttagaaagggtcttttagcctgtgtatgttttcagctgctccagcaagtcctgggctccaaagagggtatcctcagcaaagaggtcaattatcttcagagtggtggggtcggggtgggggggaccctgggcgcactccaaccagagccacctccattttgatccattctaaatgtattttatgtaagatttaattagaagaaaagggcttcttgaaatattttttgaaaaccactgctctaattgatatcctttatgataaatcacctcgaggatcttcacagtgaggtgacatgggggatgcagaaggcaggtcctcagccatggaaggtctggggaaggggcactgctgtcctgattgggacgatggaggcctggaggtgtctggatggtagaagtctttgaggcacagaaagctgccttagcagggaggtgtaagggttcctgggaggaaggtggagagcatgatcctgaggaacagggagtcttgcatcacggcaatggagggactctgattctaagggataaggatgcctgaggtttttccagagagctatggggttccatgggcaggctctgagcctgtgcccgccactaaccccactgaagcccgtccctt

SEQ ID NO: 2 - DNA, 2191 bp, synthetic construct
  source 1..2191: mol_type = other DNA
gagaattcatccgtgctctgtatgaatctgaggaaaactgcgaggtagaccctatcaagtgcacagcatccagtttggcagagcaccaggccaacctgcgaatgtgctgtgagttggccctgtgcaaggtggtcaactcccactggtgagactgggaacgctgggctggggggccagggtcgggggaattatgtgttcatctgttcatctatctgtccatcctcaaagaggactgagcaccatttatgggcaaagcattgttctaggcgctatagagcaaacaggtgaaagaggcctggtccctgccctcagagggcctccaccagaatggggacaaattagaagaaaaaaaaaaaaagccacagagccataatggtgtgtaagtgctgagtaagggtccccccaacctctgtgtgacataaggtcagagagaaggcagagctttgagataagtggggaagaggtgcccccttgggtaggctttgaagactggtttaggttctgatatatggacatagttggcaagaaagacatttcagaagaaggctgtgagaaaggcacatgtgtgatggtgaaaaggcccaggagttttcaggggacattaaagtaggttagtagcaattacatcaggtttagtggagcatgtgcctcataatggggagtggcgggagagatgtctgggcaggaagattagctttagaaactggaaggcctcaaggagtctgaggtcattggtaggccttgggatgccattaaaggtgtcagaaatattgtgatttagaagattaatccataggctgggcacggtggctcacacctgtaataccagcactttgggaggctgaggcgggcagaccacctgaggtcaggagtttgagaccaggctgaccaacatggagaaaccctgtctctactaaaaatacaaaattagccagatgtggtggcacatgcctgtaatcccagctattcgggaggctaaggcaggagaatcacttgaatctgggaggtggaggttgtagtgagccgagatcacacgattgcactctagcctgggcaacaagagtgaaactccatctcaaaaaaaaaaaattaatccataagtagagtgtatgtagtagagtagagggatgtatgttgggggatgactggaatagagccagctgggggctcggaggcatgaaagtcagatcctgaatcagaacagtgacagaagttaggaaggagctactggaaggaccctctcgggaaaggatcagtaggaattgtcagcttattggaaaggagggagcatgtctgtggggagtcacggatgactcaagaggccatgaggctggtggttgggagaccggtggcctcattgacaaccaggaaagtcaggaggaggagccagttggaggtgtgggggcgatggtgagctctgctttacaccagccgagtttaaggtgtcagtgggacattgaagtggaactgggaggtgcggggagtgagctctgagccattccagggactggggatcatgcctggggcacctccatccccatttccctggaatccagaagagttggggggtccgagctccctgtacctcaagtgaccctccatctctctcccatctctgtctctccctggtgtctgtttttcttctcctcctctccttgtctctctcccacacccctccatctctctcccacgtgtctctcccctcaccttctctccccctccatttctctctccctaatctgtctgttccctctgccatggcccccttcttcaagcagcctcccatcttgctcctgcggtccctccttccctgtctctctcacccctgtttccacaccctcacctcctaccacccccctcagcatgttccctggaagctgagggtctctggggctcagtcccggtctctctctttctctctctctctctctgtctccccgacccttccccccagcgtgttcccgagggagctgaaggaggtgtttgcttcgtggcggctgcgctgcgcagagcgaggccgggaggacatcgcagacaggcttatcagcgcctcactcttcctgcgcttcctctgcccagcgattatgtcgcccagtctctttgggcttatgcaggagtacccagatgagcagacctcacgaaccctcaccctcattgccaaggtcatccagaacctggccaacttttccaa

SEQ ID NO: 3 - DNA, 3950 bp, synthetic construct
  source 1..3950: mol_type = other DNA
atgctggtggaggaggagctgcgccgggaccaccccgccatggctgagccgctgccagaacccaagaagaggctgctcgacgctcaggtggaaattacaatgtcatttatcttctccgtgtcccatccccatccatcccactgtctttcgtgcactcactacaccagccacctagccccatcaccatctgtctctcatagtctgctgtttgtccactggctgctcctggcagccccctagtgaccccatcttcatcccatcgtctgtgcctttgtcactcctggcagcgtcagcccaactcctgtgccttcccatccagtcttcccactcctctctgcatctcaggaccttctctaccagaaccttggtctttctgcccctagaccccacctagttccaagaacccctgccccttctttgctcactcctattcaagccacgttgttcagcttcctctgcgctcttgggccagagggctagaagctgccgttttctggaatagagcacagggcagtatgatctgtagtttctccaggccctggccggtaccctgaaaacttggggacccatcacctctgttctcttggctccctaattttcctgtctccttggcagctcctgcatagcttcctcttcctgactcttcagatcttgaaggccttccatcctgtaacctccctttgccctcagtatttaagtccagcctccctctggcctccctcccactctggccctcagaccttcccagctgcctgctgcccagcctctcttctcacaagccagcttctaggacctcccttctgcacccttaccccttgctttcccaaaattctgctcattttcctacccatactcctctttgctctgactgctaggctccccccgcctgccatccccccaccaaggctcctgaccccatgaccccactctctcccactgcagctcctcatcaggtaattctcctggttccgctttggccacgggcggaggacacagggggaggtgactccggaccactgcaggttggtcgtgaagcccactccctccaacacctccggagcctctcccctctcactgctgccctccacacccagagaacctccacagactccagccctccgacacctgcacagatccatctcccaagacaccacccaaagagagcatttgctgctgcttcccagaactgtccaacaataccttagcaacaccaagagttgggccctagatgggcccagcacattcacaggtcacacccacttccctgcaaaacccaccccctcccagcctcctcctgactctaagccctcctcttcctctacctctccagtgtatgtctgtcaccccccatttcaccagagcgtccttaggggctgggggtgggtttgttaatggggtggaggcaatgatgggttggaggatcttggctataggggctgtgctgactgcagcaggtaggttgggtttccctcttccttccctaatcttggttctctaccctcctttccactcctcacctgattctctctcttcctcctccttatatctgtgaggcagaaggcatctgaagctcatattagcccccattgggtgggaattaggagtgggtagttaactcagggagacttgagataccctggaaaaaatgctattgagatgtcctgacattaggcagggtggatggaacaagaaggagcaagaaaggaacctcaggcagatgttaggacatggacttgatcatgtggcctgggagtttagaaatggggagagacatcctcctagatcagatcgtgggctcagtaggcatgttgattcccagggagaggtgccaggaacagcatggtaaagaatgtactcttcacagctcacatccccaggttgctgatgccactcactccccctctcctgccatcgagtggccttgccggacacatcaccctacctaaaaagccagtaaatgagaacctgtcagctatagccatcatttctgagatgcgattttctttgggattgagctgcagtgggcagtggctccttacactgtaattttaattctctgcctgcccagcctctctgtcaaagtagctggtgatctataaagatgctaaaaggcaccaggggactttgccatttaaaggactcctgcagtgaattcttttgtaaaatgaataatggcaccctaatttatccactttctaaatttgggtccatgggggtgtccagggcatgcttatgtgctgtcaccagcagacaaacagagggaatggaatctgggggttccttccctgctctcccgccatactcaggataccctaccataagtgatttcctctcactgacttgcagaaaatgtgtgagatacccagcaagctaagaaggcagttttgctgggtatctcatacccaaggctggggtttgggtgatctgagaggttagctccttgatcctaggatggaagggagagcttatatagaagcttttacttggaaggttttgtatcctaaggtcagacatagctatattaccaagcctaaatgccatgtggcccaggaaataatttggacatttgttctaaaccacttgtggtaggtattggtctctctgcaactcagccattaattagaaattagttttgagcctgaattttaaaaagccaagtgttgcccccagcccacacacacacacacggacatgtacagtacaaaccccagataattacaacagccaaagagagaaggaagtgaatttcccaaccagaagcgtagggaaattcagatggctttcttttctcccagcagaggaacagaaggcggagctaagggcaggaaccaggagttggtcaaggagctataggaggtgatgagagtagaaccaggggtaggagctggtctggtacccctcaccctctaattgggagcccagggagaaggactgaaaagaagatgggagtggaaaagaataaagccagtttctgcttcccagggatgcagagattggggcatgctgtgtctgcagaagctcctagtcatttccgccataattgtgagagagaggggcagccctcccacaagatttttcccttcccatatcacttccctgaatccccttcctttccccccagtacagttaaacctctctctaatttggaatgttatatttgagaagatggccactgtgaataagtgacaaacggaatgacagtgtctatttaatgaaatgcatgtcttcaaaatatatacagaactcgatgaacaaggctttttccactcctcagggagcatgcattaatgaatagataggattcaaaagtctgttttctggtatgggttaaatatcccctcctacagacatatttccaccactaatttgcttagtacaccttttcttcacagataaaggaaaatgcaagctcagtttttcttcagattatgaagaaattccaaatccacaggggtttggattaatgaggttttgctgtactgcctccccttattcctcaacatgaagttcccacctcggattggggatgggtgggagggggtttcaagaggaggagggtgggatgggcaaggaatatacacaggtgaagccagagaagggttaggttgggggtgcggtgggaacttgctgttttgatctggtttcctggtgtgacactctgggttaaaggcttgaaggcccctgttaggagtctaggggtgagattctcttctctctgatcccagaggacgttaacttctactgcaggtgagaaacaaaataggaggatggtggggactgtcctgggaggagggggtggtccatggcttgtggtgtgggctggctataggggaggccactgctaggggactggcatccaggcccccttgaagcgtctcaataagtccgcgctctcctttttggtgtcttgcaggagaggcagcttccccccttgggtccaacaaacccgc

SEQ ID NO: 4 - DNA, 60 bp, Homo sapiens
  source 1..60: mol_type = other DNA
gctcaggtggaaattacaatgtcatttatcttctccgtgtcccatccccatccatcccac

SEQ ID NO: 5 - DNA, 12 bp, Homo sapiens
  source 1..12: mol_type = other DNA
gctcaggtggaa

SEQ ID NO: 6 - DNA, 43 bp, Homo sapiens
  source 1..43: mol_type = other DNA
ttgcaggagaggcagcttccccccttgggtccaacaaacccgc

SEQ ID NO: 7 - DNA, 12 bp, Homo sapiens
  source 1..12: mol_type = other DNA
ttgcaggagagg

SEQ ID NO: 8 - DNA, 20 bp, synthetic construct
  source 1..20: mol_type = other DNA
ggctgcttgaagaagggggc

SEQ ID NO: 9 - DNA, 20 bp, synthetic construct
  source 1..20: mol_type = other DNA
caagatgggaggctgcttga

SEQ ID NO: 10 - DNA, 20 bp, synthetic construct
  source 1..20: mol_type = other DNA
aggagcaagatgggaggctg

SEQ ID NO: 11 - DNA, 20 bp, synthetic construct
  source 1..20: mol_type = other DNA
accgcaggagcaagatggga

SEQ ID NO: 12 - DNA, 20 bp, synthetic construct
  source 1..20: mol_type = other DNA
agaccctcagcttccaggga

SEQ ID NO: 13 - DNA, 20 bp, synthetic construct
  source 1..20: mol_type = other DNA
aaacacctccttcagctccc

SEQ ID NO: 14 - DNA, 20 bp, synthetic construct
  source 1..20: mol_type = other DNA
gggcttcagtggggttagtg

SEQ ID NO: 15 - DNA, 20 bp, synthetic construct
  source 1..20: mol_type = other DNA
gacgggcttcagtggggtta

SEQ ID NO: 16 - DNA, 20 bp, synthetic construct
  source 1..20: mol_type = other DNA
ctgaagggacgggcttcagt

SEQ ID NO: 17 - DNA, 20 bp, synthetic construct
  source 1..20: mol_type = other DNA
ttgtaatttccacctgagcg

SEQ ID NO: 18 - DNA, 20 bp, synthetic construct
  source 1..20: mol_type = other DNA
ggctcaggcagcggctcagc

SEQ ID NO: 19 - DNA, 20 bp, synthetic construct
  source 1..20: mol_type = other DNA
aatgacattgtaatttccac

SEQ ID NO: 20 - DNA, 20 bp, synthetic construct
  source 1..20: mol_type = other DNA
agataaatgacattgtaatt

SEQ ID NO: 21 - DNA, 20 bp, synthetic construct
  source 1..20: mol_type = other DNA
ggagaagataaatgacattg

SEQ ID NO: 22 - DNA, 20 bp, synthetic construct
  source 1..20: mol_type = other DNA
gatgggacacggagaagata

SEQ ID NO: 23 - DNA, 20 bp, synthetic construct
  source 1..20: mol_type = other DNA
gatggatggggatgggacac

SEQ ID NO: 24 - DNA, 20 bp, synthetic construct
  source 1..20: mol_type = other DNA
aagacagtgggatggatggg

SEQ ID NO: 25 - DNA, 20 bp, synthetic construct
  source 1..20: mol_type = other DNA
gagtgcacgaaagacagtgg

SEQ ID NO: 26 - DNA, 20 bp, synthetic construct
  source 1..20: mol_type = other DNA
ctggtgtagtgagtgcacga

SEQ ID NO: 27 - DNA, 20 bp, synthetic construct
  source 1..20: mol_type = other DNA
ggctaggtggctggtgtagt

SEQ ID NO: 28 - DNA, 20 bp, synthetic construct
  source 1..20: mol_type = other DNA
gatgaggagctgcagtggga

SEQ ID NO: 29 - DNA, 20 bp, synthetic construct
  source 1..20: mol_type = other DNA
aattacctgatgaggagctg

SEQ ID NO: 30 - DNA, 20 bp, synthetic construct
  source 1..20: mol_type = other DNA
accaggagaattacctgatg

SEQ ID NO: 31 - DNA, 18 bp, synthetic construct
  source 1..18: mol_type = other DNA
cctccgcccgtggccaaa

SEQ ID NO: 32 - DNA, 18 bp, synthetic construct
  source 1..18: mol_type = other DNA
cctgtgtcctccgcccgt

SEQ ID NO: 33 - DNA, 19 bp, synthetic construct
  source 1..19: mol_type = other DNA
tggtccggagtcacctccc

SEQ ID NO: 34 - DNA, 19 bp, synthetic construct
  source 1..19: mol_type = other DNA
ctgcagtggtccggagtca

SEQ ID NO: 35 - DNA, 20 bp, synthetic construct
  source 1..20: mol_type = other DNA
cctagcagtggcctccccta

SEQ ID NO: 36 - DNA, 20 bp, synthetic construct
  source 1..20: mol_type = other DNA
atgccagtcccctagcagtg

SEQ ID NO: 37 - DNA, 20 bp, synthetic construct
  source 1..20: mol_type = other DNA
ctgcctctcctgcaagacac

SEQ ID NO: 38 - DNA, 19 bp, synthetic construct
  source 1..19: mol_type = other DNA
ccaaggggggaagctgcct

SEQ ID NO: 39 - DNA, 19 bp, synthetic construct
  source 1..19: mol_type = other DNA
ggtttgttggacccaaggg

SEQ ID NO: 40 - DNA, 12 bp, Homo sapiens
  source 1..12: mol_type = other DNA
gtacctgtaagc

SEQ ID NO: 41 - DNA, 12 bp, Homo sapiens
  source 1..12: mol_type = other DNA
tctcaggatgtg

SEQ ID NO: 42 - DNA, 12 bp, Homo sapiens
  source 1..12: mol_type = other DNA
agccaggtacct

SEQ ID NO: 43 - DNA, 12 bp, Homo sapiens
  source 1..12: mol_type = other DNA
gaagaggtggaa

SEQ ID NO: 44 - DNA, 12 bp, Homo sapiens
  source 1..12: mol_type = other DNA
ccccaggatcag

SEQ ID NO: 45 - DNA, 12 bp, Homo sapiens
  source 1..12: mol_type = other DNA
gcccaggtggtg

SEQ ID NO: 46 - DNA, 12 bp, Homo sapiens
  source 1..12: mol_type = other DNA
cctcaggttcta

SEQ ID NO: 47 - DNA, 12 bp, Homo sapiens
  source 1..12: mol_type = other DNA
gatcaggtactc

SEQ ID NO: 48 - DNA, 12 bp, Homo sapiens
  source 1..12: mol_type = other DNA
tctcaggatggg

SEQ ID NO: 49 - DNA, 12 bp, Homo sapiens
  source 1..12: mol_type = other DNA
agaaaagtgagg

SEQ ID NO: 50 - DNA, 12 bp, Homo sapiens
  source 1..12: mol_type = other DNA
ttcaagaactag

SEQ ID NO: 51 - DNA, 15 bp, synthetic construct
  source 1..15: mol_type = other DNA
gctcaggtggaaatt

SEQ ID NO: 52 - DNA, 13 bp, synthetic construct
  source 1..13: mol_type = other DNA
ttgcaggagaggc

SEQ ID NO: 53 - DNA, 13 bp, synthetic construct
  source 1..13: mol_type = other DNA
gctcaggagaggc

SEQ ID NO: 54 - DNA, 12 bp, synthetic construct
  source 1..12: mol_type = other DNA
gctcagagaggc

SEQ ID NO: 55 - DNA, 15 bp, synthetic construct
  source 1..15: mol_type = other DNA
gctcaggtggagagg

SEQ ID NO: 56 - DNA, 189 bp, synthetic construct
  source 1..189: mol_type = other DNA
gtgcccgccactaaccccactgaagcccgtcccttcaggctgatgctggtggaggaggagctgcgccgggaccaccccgccatggctgagccgctgccagaacccaagaggctgctcgacgctcaggtggaaattacaatgtcatttatcttctccgtgtcccatccccatccatcccactgtctttcg

SEQ ID NO: 57 - DNA, 189 bp, Homo sapiens; identical to SEQ ID NO: 56
  source 1..189: mol_type = genomic DNA

SEQ ID NO: 58 - DNA, 189 bp, Macaca mulatta; identical to SEQ ID NO: 56
  source 1..189: mol_type = genomic DNA

SEQ ID NO: 59 - DNA, 189 bp, Mus musculus
  source 1..189: mol_type = genomic DNA
gtgcccgccactaaccccactgaagcccgtcccttcaggctgatgctggtggaggaggagctgcgtcgggaccaccccgccatggctgagccgctgcctgagcccaagaggctgctcgacgctcaggtggaaattacaatgtcatttatcttctccgtgtcccatccccatccatcccactgtctttcg

SEQ ID NO: 60 - DNA, 189 bp, Rattus rattus
  source 1..189: mol_type = genomic DNA
gtgcccgccactaaccccactgaagcccgtcccttcaggctgatgctggtggaggaggagctgcgccgggaccaccccgccatggctgagccgctgcctgaacccaagaggctgctcgacgctcaggtggaaattacaatgtcatttatcttctccgtgtcccatccccatccatcccactgtctttcg

SEQ ID NO: 61 - DNA, 189 bp, Sus domesticus
  source 1..189: mol_type = genomic DNA
gtgcccgccactaaccccactgaagcccgtcccttcaggctgatgctggtggaggaggagctgcgccgggaccaccctgccatggctgagccgctgcccgaacccaagaggctgctcgacgctcaggtggaaattacaatgtcatttatcttctccgtgtcccatccccatccatcccactgtctttcg

SEQ ID NO: 62 - DNA, 189 bp, Bos taurus
  source 1..189: mol_type = genomic DNA
gtgcccgccactaaccccactgaagcccgtcccttcaggctgatgctggtggaggaggagctgcgccgggaccaccccaccatggctgagccgctgccagaacccaagaggctgctcgacgctcaggtggaaattacaatgtcatttatcttctccgtgtcccatccccatccatcccactgtctttcg

SEQ ID NO: 63 - DNA, 189 bp, Felis catus
  source 1..189: mol_type = genomic DNA
gtgcccgccactaaccccactgaagcccgtcccttcaggctgatgctggtggaggaggagctgcgccgggaccaccccgccatggctgagccgctgcccgaacccaagaggctgctcgacgctcaggtggaaattacaatgtcatttatcttctccgtgtcccatccccatccatcccactgtctttcg

SEQ ID NO: 64 - DNA, 189 bp, Canis lupus; identical to SEQ ID NO: 60
  source 1..189: mol_type = genomic DNA

SEQ ID NO: 65 - DNA, 189 bp, unidentified
  source 1..189: mol_type = genomic DNA
gtgcccgccactaaccccactgaagcccgtcccttcaggctgatgctggtggaggaggagctgcgccgggaccaccccaccatggctgagccgctgcctgaacccaagaggctgctcgacgctcaggtggaaattacaatgtcatttatcttctccgtgtcccatccccatccatcccactgtctttcg

SEQ ID NO: 66 - DNA, 185 bp, Ornithorhynchus anatinus
  source 1..185: mol_type = genomic DNA
gagttttagcgctgacatggcttctgtttcctcaggttaatgtctgtggaggaagagttaaagaaagaccacgctgagatgcaggcggccgtggattccaaacagaagattatcgatgctcaggttgggggtcgttaccattcgtcctccctcccctcactctcctcacctcccccccttcttct

SEQ ID NO: 67 - protein, 30 aa, synthetic construct
LMLVEEELRRDHPAMAEPLPEPKKRLLDAD

SEQ ID NO: 68 - protein, 20 aa, synthetic construct
PSLQADRGGGQAASGPPKHG

SEQ ID NO: 69 - DNA, 27 bp, Homo sapiens
  source 1..27: mol_type = other DNA
cccactgcagctcctcatcaggtaatt

SEQ ID NO: 70 - DNA, 20 bp, Homo sapiens
  source 1..20: mol_type = other DNA
cccactgaagcccgtccctt

SEQ ID NO: 71 - DNA, 19 bp, synthetic construct
  source 1..19: mol_type = other DNA
tcgagcagcctcttcttgg

SEQ ID NO: 72 - DNA, 19 bp, synthetic construct
  source 1..19: mol_type = other DNA
cgtcgagcagcctcttctt

SEQ ID NO: 73 - DNA, 19 bp, synthetic construct
  source 1..19: mol_type = other DNA
gagcgtcgagcagcctctt

SEQ ID NO: 74 - DNA, 18 bp, synthetic construct
  source 1..18: mol_type = other DNA
tgagcgtcgagcagcctc

SEQ ID NO: 75 - DNA, 18 bp, synthetic construct
  source 1..18: mol_type = other DNA
acctgagcgtcgagcagc

SEQ ID NO: 76 - DNA, 20 bp, synthetic construct
  source 1..20: mol_type = other DNA
aatttccacctgagcgtcga

SEQ ID NO: 77 - DNA, 20 bp, synthetic construct
  source 1..20: mol_type = other DNA
tgtaatttccacctgagcgt

SEQ ID NO: 78 - DNA, 22 bp, synthetic construct
  source 1..22: mol_type = other DNA
tgacattgtaatttccacctga

SEQ ID NO: 79 - DNA, 22 bp, synthetic construct
  source 1..22: mol_type = other DNA
gataaatgacattgtaatttcc

SEQ ID NO: 80 - DNA, 22 bp, synthetic construct
  source 1..22: mol_type = other DNA
ggagaagataaatgacattgta

SEQ ID NO: 81 - DNA, 20 bp, synthetic construct
  source 1..20: mol_type = other DNA
cacggagaagataaatgaca

SEQ ID NO: 82 - DNA, 20 bp, synthetic construct
  source 1..20: mol_type = other DNA
gacacggagaagataaatga

SEQ ID NO: 83 - DNA, 20 bp, synthetic construct
  source 1..20: mol_type = other DNA
ggacacggagaagataaatg

SEQ ID NO: 84 - DNA, 20 bp, synthetic construct
  source 1..20: mol_type = other DNA
gggacacggagaagataaat

SEQ ID NO: 85 - DNA, 20 bp, synthetic construct
  source 1..20: mol_type = other DNA
tgggacacggagaagataaa

SEQ ID NO: 86 - DNA, 22 bp, synthetic construct
  source 1..22: mol_type = other DNA
gatgggacacggagaagataaa

SEQ ID NO: 87 - DNA, 20 bp, synthetic construct
  source 1..20: mol_type = other DNA
atgggacacggagaagataa

SEQ ID NO: 88 - DNA, 21 bp, synthetic construct
  source 1..21: mol_type = other DNA
gatgggacacggagaagataa

SEQ ID NO: 89 - DNA, 21 bp, synthetic construct
  source 1..21: mol_type = other DNA
ggatgggacacggagaagata

SEQ ID NO: 90 - DNA, 22 bp, synthetic construct
  source 1..22: mol_type = other DNA
gggatgggacacggagaagata

SEQ ID NO: 91 - DNA, 20 bp, synthetic construct
  source 1..20: mol_type = other DNA
ggatgggacacggagaagat

SEQ ID NO: 92 - DNA, 19 bp, synthetic construct
  source 1..19: mol_type = other DNA
ggatgggacacggagaaga

SEQ ID NO: 93 - DNA, 18 bp, synthetic construct
  source 1..18: mol_type = other DNA
ggatgggacacggagaag

SEQ ID NO: 94 - DNA, 18 bp, synthetic construct
  source 1..18: mol_type = other DNA
gggatgggacacggagaa

SEQ ID NO: 95 - DNA, 19 bp, synthetic construct
  source 1..19: mol_type = other DNA
atgggacacggagaagata

SEQ ID NO: 96 - DNA, 19 bp, synthetic construct
  source 1..19: mol_type = other DNA
gatgggacacggagaagat

SEQ ID NO: 97 - DNA, 18 bp, synthetic construct
  source 1..18: mol_type = other DNA
tgggacacggagaagata

SEQ ID NO: 98 - DNA, 18 bp, synthetic construct
  source 1..18: mol_type = other DNA
atgggacacggagaagat

SEQ ID NO: 99 - DNA, 18 bp, synthetic construct
  source 1..18: mol_type = other DNA
gatgggacacggagaaga

SEQ ID NO: 100 - DNA, 17 bp, synthetic construct
  source 1..17: mol_type = other DNA
gggacacggagaagata

SEQ ID NO: 101 - DNA, 17 bp, synthetic construct
  source 1..17: mol_type = other DNA
tgggacacggagaagat

SEQ ID NO: 102 - DNA, 17 bp, synthetic construct
  source 1..17: mol_type = other DNA
atgggacacggagaaga

SEQ ID NO: 103 - DNA, 17 bp, synthetic construct
  source 1..17: mol_type = other DNA
gatgggacacggagaag

SEQ ID NO: 104 - DNA, 16 bp, synthetic construct
  source 1..16: mol_type = other DNA
ggacacggagaagata

SEQ ID NO: 105 - DNA, 16 bp, synthetic construct
  source 1..16: mol_type = other DNA
gggacacggagaagat

SEQ ID NO: 106 - DNA, 16 bp, synthetic construct
  source 1..16: mol_type = other DNA
tgggacacggagaaga

SEQ ID NO: 107 - DNA, 16 bp, synthetic construct
  source 1..16: mol_type = other DNA
atgggacacggagaag

SEQ ID NO: 108 - DNA, 16 bp, synthetic construct
  source 1..16: mol_type = other DNA
gatgggacacggagaa

SEQ ID NO: 109 - DNA, 15 bp, synthetic construct
  source 1..15: mol_type = other DNA
gacacggagaagata

SEQ ID NO: 110 - DNA, 15 bp, synthetic construct
  source 1..15: mol_type = other DNA
ggacacggagaagat

SEQ ID NO: 111 - DNA, 15 bp, synthetic construct
  source 1..15: mol_type = other DNA
gggacacggagaaga

SEQ ID NO: 112 - DNA, 15 bp, synthetic construct
  source 1..15: mol_type = other DNA
tgggacacggagaag

SEQ ID NO: 113 - DNA, 15 bp, synthetic construct
  source 1..15: mol_type = other DNA
atgggacacggagaa

SEQ ID NO: 114 - DNA, 15 bp, synthetic construct
  source 1..15: mol_type = other DNA
gatgggacacggaga

SEQ ID NO: 115 - DNA, 20 bp, Homo sapiens
  source 1..20: mol_type = other DNA
gcccccttcttcaagcagcc

SEQ ID NO: 116 - DNA, 20 bp, Homo sapiens
  source 1..20: mol_type = other DNA
tcaagcagcctcccatcttg

SEQ ID NO: 117 - DNA, 20 bp, Homo sapiens
  source 1..20: mol_type = other DNA
cagcctcccatcttgctcct

SEQ ID NO: 118 - DNA, 20 bp, Homo sapiens
  source 1..20: mol_type = other DNA
tcccatcttgctcctgcggt

SEQ ID NO: 119 - DNA, 20 bp, Homo sapiens
  source 1..20: mol_type = other DNA
tccctggaagctgagggtct

SEQ ID NO: 120 - DNA, 20 bp, Homo sapiens
  source 1..20: mol_type = other DNA
gggagctgaaggaggtgttt

SEQ ID NO: 121 - DNA, 20 bp, Homo sapiens
  source 1..20: mol_type = other DNA
cactaaccccactgaagccc

SEQ ID NO: 122 - DNA, 20 bp, Homo sapiens
  source 1..20: mol_type = other DNA
taaccccactgaagcccgtc

SEQ ID NO: 123 - DNA, 20 bp, Homo sapiens
  source 1..20: mol_type = other DNA
actgaagcccgtcccttcag

SEQ ID NO: 124 - DNA, 20 bp, Homo sapiens
  source 1..20: mol_type = other DNA
cgctcaggtggaaattacaa

SEQ ID NO: 125 - DNA, 20 bp, Homo sapiens
  source 1..20: mol_type = other DNA
gctgagccgctgcctgagcc

SEQ ID NO: 126 - DNA, 20 bp, Homo sapiens
  source 1..20: mol_type = other DNA
gtggaaattacaatgtcatt

SEQ ID NO: 127 - DNA, 20 bp, Homo sapiens
  source 1..20: mol_type = other DNA
aattacaatgtcatttatct

SEQ ID NO: 128 - DNA, 20 bp, Homo sapiens
  source 1..20: mol_type = other DNA
caatgtcatttatcttctcc

SEQ ID NO: 129 - DNA, 20 bp, Homo sapiens
  source 1..20: mol_type = other DNA
tatcttctccgtgtcccatc

SEQ ID NO: 130 - DNA, 20 bp, Homo sapiens
  source 1..20: mol_type = other DNA
gtgtcccatccccatccatc

SEQ ID NO: 131 - DNA, 20 bp, Homo sapiens
  source 1..20: mol_type = other DNA
cccatccatcccactgtctt

SEQ ID NO: 132 - DNA, 20 bp, Homo sapiens
  source 1..20: mol_type = other DNA
ccactgtctttcgtgcactc

SEQ ID NO: 133 - DNA, 20 bp, Homo sapiens
  source 1..20: mol_type = other DNA
tcgtgcactcactacaccag

SEQ ID NO: 134 - DNA, 20 bp, Homo sapiens
  source 1..20: mol_type = other DNA
actacaccagccacctagcc

SEQ ID NO: 135 - DNA, 19 bp, Homo sapiens
  source 1..19: mol_type = other DNA
ccaagaagaggctgctcga

SEQ ID NO: 136 - DNA, 19 bp, Homo sapiens
  source 1..19: mol_type = other DNA
aagaagaggctgctcgacg

SEQ ID NO: 137 - DNA, 19 bp, Homo sapiens
  source 1..19: mol_type = other DNA
aagaggctgctcgacgctc

SEQ ID NO: 138 - DNA, 18 bp, Homo sapiens
  source 1..18: mol_type = other DNA
gaggctgctcgacgctca

SEQ ID NO: 139 - DNA, 18 bp, Homo sapiens
  source 1..18: mol_type = other DNA
gctgctcgacgctcaggt

SEQ ID NO: 140 - DNA, 20 bp, Homo sapiens
  source 1..20: mol_type = other DNA
tcgacgctcaggtggaaatt

SEQ ID NO: 141 - DNA, 20 bp, Homo sapiens
  source 1..20: mol_type = other DNA
acgctcaggtggaaattaca

SEQ ID NO: 142 - DNA, 22 bp, Homo sapiens
  source 1..22: mol_type = other DNA
tcaggtggaaattacaatgtca

SEQ ID NO: 143 - DNA, 22 bp, Homo sapiens
  source 1..22: mol_type = other DNA
ggaaattacaatgtcatttatc

SEQ ID NO: 144 - DNA, 22 bp, Homo sapiens
  source 1..22: mol_type = other DNA
tacaatgtcatttatcttctcc

SEQ ID NO: 145 - DNA, 20 bp, Homo sapiens
  source 1..20: mol_type = other DNA
tgtcatttatcttctccgtg

SEQ ID NO: 146 - DNA, 20 bp, Homo sapiens
  source 1..20: mol_type = other DNA
tcatttatcttctccgtgtc

SEQ ID NO: 147 - DNA, 20 bp, Homo sapiens
  source 1..20: mol_type = other DNA
catttatcttctccgtgtcc

SEQ ID NO: 148 - DNA, 20 bp, Homo sapiens
  source 1..20: mol_type = other DNA
atttatcttctccgtgtccc

SEQ ID NO: 149 - DNA, 20 bp, Homo sapiens
  source 1..20: mol_type = other DNA
tttatcttctccgtgtccca

SEQ ID NO: 150 - DNA, 22 bp, Homo sapiens
  source 1..22: mol_type = other DNA
tttatcttctccgtgtcccatc

SEQ ID NO: 151 - DNA, 20 bp, Homo sapiens
  source 1..20: mol_type = other DNA
ttatcttctccgtgtcccat

SEQ ID NO: 152 - DNA, 21 bp, Homo sapiens
  source 1..21: mol_type = other DNA
ttatcttctccgtgtcccatc

SEQ ID NO: 153 - DNA, 21 bp, Homo sapiens
  source 1..21: mol_type = other DNA
tatcttctccgtgtcccatcc

SEQ ID NO: 154 - DNA, 22 bp, Homo sapiens
  source 1..22: mol_type = other DNA
tatcttctccgtgtcccatccc

SEQ ID NO: 155 - DNA, 20 bp, Homo sapiens
  source 1..20: mol_type = other DNA
atcttctccgtgtcccatcc

SEQ ID NO: 156 - DNA, 19 bp, Homo sapiens
  source 1..19: mol_type = other DNA
tcttctccgtgtcccatcc

SEQ ID NO: 157 - DNA, 18 bp, Homo sapiens
  source 1..18: mol_type = other DNA
cttctccgtgtcccatcc

SEQ ID NO: 158 - DNA, 18 bp, Homo sapiens
  source 1..18: mol_type = other DNA
ttctccgtgtcccatccc

SEQ ID NO: 159 - DNA, 19 bp, Homo sapiens
  source 1..19: mol_type = other DNA
tatcttctccgtgtcccat

SEQ ID NO: 160 - DNA, 19 bp, Homo sapiens
  source 1..19: mol_type = other DNA
atcttctccgtgtcccatc

SEQ ID NO: 161 - DNA, 18 bp, Homo sapiens
  source 1..18: mol_type = other DNA
tatcttctccgtgtccca

SEQ ID NO: 162 - DNA, 18 bp, Homo sapiens
  source 1..18: mol_type = other DNA
atcttctccgtgtcccat

SEQ ID NO: 163 - DNA, 18 bp, Homo sapiens
  source 1..18: mol_type = other DNA
tcttctccgtgtcccatc

SEQ ID NO: 164 - DNA, 17 bp, Homo sapiens
  source 1..17: mol_type = other DNA
tatcttctccgtgtccc

SEQ ID NO: 165 - DNA, 17 bp, Homo sapiens
  source 1..17: mol_type = other DNA
atcttctccgtgtccca

SEQ ID NO: 166 - DNA, 17 bp, Homo sapiens
  source 1..17: mol_type = other DNA
tcttctccgtgtcccat

SEQ ID NO: 167 - DNA, 17 bp, Homo sapiens
  source 1..17: mol_type = other DNA
cttctccgtgtcccatc

SEQ ID NO: 168 - DNA, 16 bp, Homo sapiens
  source 1..16: mol_type = other DNA
tatcttctccgtgtcc

SEQ ID NO: 169 - DNA, 16 bp, Homo sapiens
  source 1..16: mol_type = other DNA
atcttctccgtgtccc

SEQ ID NO: 170 - DNA, 16 bp, Homo sapiens
  source 1..16: mol_type = other DNA
tcttctccgtgtccca

SEQ ID NO: 171 - DNA, 16 bp, Homo sapiens
  source 1..16: mol_type = other DNA
cttctccgtgtcccat

SEQ ID NO: 172 - DNA, 16 bp, Homo sapiens
  source 1..16: mol_type = other DNA
ttctccgtgtcccatc

SEQ ID NO: 173 - DNA, 15 bp, Homo sapiens
  source 1..15: mol_type = other DNA
tatcttctccgtgtc

SEQ ID NO: 174 - DNA, 15 bp, Homo sapiens
  source 1..15: mol_type = other DNA
atcttctccgtgtcc

SEQ ID NO: 175 - DNA, 15 bp, Homo sapiens
  source 1..15: mol_type = other DNA
tcttctccgtgtccc

SEQ ID NO: 176 - DNA, 15 bp, Homo sapiens
  source 1..15: mol_type = other DNA
cttctccgtgtccca

SEQ ID NO: 177 - DNA, 15 bp, Homo sapiens
  source 1..15: mol_type = other DNA
ttctccgtgtcccat

SEQ ID NO: 178 - DNA, 15 bp, Homo sapiens
  source 1..15: mol_type = other DNA
tctccgtgtcccatc

SEQ ID NO: 179 - DNA, 20 bp, Homo sapiens
  source 1..20: mol_type = other DNA
tcccactgcagctcctcatc

SEQ ID NO: 180 - DNA, 20 bp, Homo sapiens
  source 1..20: mol_type = other DNA
cagctcctcatcaggtaatt

SEQ ID NO: 181 - DNA, 20 bp, Homo sapiens
  source 1..20: mol_type = other DNA
catcaggtaattctcctggt

SEQ ID NO: 182 - DNA, 18 bp, Homo sapiens
  source 1..18: mol_type = other DNA
tttggccacgggcggagg

SEQ ID NO: 183 - DNA, 18 bp, Homo sapiens
  source 1..18: mol_type = other DNA
acgggcggaggacacagg

SEQ ID NO: 184 - DNA, 19 bp, Homo sapiens
  source 1..19: mol_type = other DNA
gggaggtgactccggacca

SEQ ID NO: 185 - DNA, 19 bp, Homo sapiens
  source 1..19: mol_type = other DNA
tgactccggaccactgcag

SEQ ID NO: 186 - DNA, 20 bp, Homo sapiens
  source 1..20: mol_type = other DNA
taggggaggccactgctagg

SEQ ID NO: 187 - DNA, 20 bp, Homo sapiens
  source 1..20: mol_type = other DNA
cactgctaggggactggcat

SEQ ID NO: 188 - DNA, 20 bp, Homo sapiens
  source 1..20: mol_type = other DNA
gtgtcttgcaggagaggcag

SEQ ID NO: 189 - DNA, 19 bp, Homo sapiens
  source 1..19: mol_type = other DNA
aggcagcttccccccttgg

SEQ ID NO: 190 - DNA, 19 bp, Homo sapiens
  source 1..19: mol_type = other DNA
cccttgggtccaacaaacc

SEQ ID NO: 191 - DNA, 38 bp, synthetic construct
  source 1..38: mol_type = other DNA
ctcgacgctcaggtggaaattacaatgtcatttatctt

SEQ ID NO: 192 - DNA, 12 bp, synthetic construct
  source 1..12: mol_type = other DNA
gcccaggtggaa

SEQ ID NO: 193 - DNA, 12 bp, synthetic construct
  source 1..12: mol_type = other DNA
gctgaggtggaa

SEQ ID NO: 194 - DNA, 12 bp, synthetic construct
  source 1..12: mol_type = other DNA
gctccggtggaa

SEQ ID NO: 195 - DNA, 12 bp, synthetic construct
  source 1..12: mol_type = other DNA
gctcatgtggaa

SEQ ID NO: 196 - DNA, 12 bp, synthetic construct
  source 1..12: mol_type = other DNA
gctcagatggaa

SEQ ID NO: 197 - DNA, 12 bp, synthetic construct
  source 1..12: mol_type = other DNA
gctcaggcggaa

SEQ ID NO: 198 - DNA, 12 bp, synthetic construct
  source 1..12: mol_type = other DNA
gctcaggtagaa

SEQ ID NO: 199 - DNA, 12 bp, synthetic construct
  source 1..12: mol_type = other DNA
gctcaggtgcaa

SEQ ID NO: 200 - DNA, 32 bp, synthetic construct
  source 1..32: mol_type = other DNA
ctcgacgctcaggtggaaattacaatgtcatt

SEQ ID NO: 201 - DNA, 32 bp, synthetic construct; identical to SEQ ID NO: 200
  source 1..32: mol_type = other DNA

SEQ ID NO: 202 - DNA, 21 bp, synthetic construct
  source 1..21: mol_type = other DNA
ctcgacgctcaggtggaaatt

(19) United States
(12) Patent Application Publication
(10) Pub. No.: US 2026/0168040 A1
(43) Pub. Date: Jun. 18, 2026

(54) AGENTS FOR MODULATING SYNGAP1 SPLICING

(71) Applicant: The Johns Hopkins University, Baltimore, MD (US)

(72) Inventors: Ingie Hong, North Bethesda, MD (US); Yoichi Araki, Cockeysville, MD (US); Richard C. Johnson, Baltimore, MD (US); Richard Huganir, Cockeysville, MD (US); Yinuo Han, Baltimore, MD (US)

(21) Appl. No.: 18/854,135

(22) PCT Filed: Apr. 5, 2023
(86) PCT No.: PCT/US2023/065370

§ 371 Date: Oct. 4, 2024

Related U.S. Application Data
(60) Provisional application No. 63/327,570, filed on Apr. 5, 2022.

(51) Int. Cl. C12Q 1/6897; A61P 25/00; C12N 15/113

(52) U.S. Cl. CPC C12Q 1/6897; A61P 25/00; C12N 15/113; C12N 2310/11; C12N 2320/33

(57) ABSTRACT

This document relates to splice reporter nucleic acid constructs (e.g., splice reporter plasmids). For example, splice reporter nucleic acid constructs that can be used to (e.g., are designed to) screen for agents (e.g., anti-sense oligonucleotides (ASOs)) that can modulate SYNGAP1 splicing are provided. Also provided herein are methods and materials for modulating SYNGAP1 splicing. For example, agents that can modulate SYNGAP1 splicing (e.g., splice-switching ASOs (SSOs)) can be administered to a mammal (e.g., a human) having, or at risk of developing, a SYNGAP1-associated neurodevelopmental disorders (NDD) to increase expression of a SynGAP1-α1 polypeptide and/or a SynGAP1-α2 polypeptide in cells within that mammal (e.g., to treat the mammal).

### CROSS-REFERENCE TO RELATED APPLICATIONS

This application is a U.S. National Stage application under 35 U.S.C. § 371 of International Application No. PCT/US2023/065370 filed on Apr. 5, 2023, which claims the benefit of U.S. Patent Application Ser. No. 63/327,570, filed on Apr. 5, 2022. The disclosures of the prior applications are considered part of, and are incorporated by reference in, the disclosure of this application.

### STATEMENT REGARDING FEDERAL FUNDING

This invention was made with government support under MH112151 awarded by the National Institutes of Health. The government has certain rights in the invention.

### SEQUENCE LISTING

This application contains a Sequence Listing that has been submitted electronically as an XML file named “44807-0417US1_SL_ST26.XML.” The XML file, created on Oct. 10, 2024, is 187,085 bytes in size. The material in the XML file is hereby incorporated by reference in its entirety.

### TECHNICAL FIELD

This document relates to splice reporter nucleic acid constructs (e.g., splice reporter plasmids). For example, this document provides splice reporter nucleic acid constructs that can be used to (e.g., are designed to) screen for agents (e.g., anti-sense oligonucleotides (ASOs)) that can modulate SYNGAP1 splicing. Also provided herein are methods and materials for modulating SYNGAP1 splicing. For example, agents that can modulate SYNGAP1 splicing (e.g., splice-switching ASOs (SSOs)) can be administered to a mammal (e.g., a human) to increase expression of a SynGAP1-α1 polypeptide and/or a SynGAP1-α2 polypeptide. For example, one or more agents that can modulate SYNGAP1 splicing (e.g., one or more SSOs) can be administered to a mammal (e.g., a human) to increase expression of a SynGAP1-α1 polypeptide by reducing or eliminating expression of a SynGAP1-β polypeptide, a SynGAP1-γ polypeptide, a SynGAP1-α3 polypeptide, and/or a SynGAP1-α2 polypeptide.

### BACKGROUND INFORMATION

SYNGAP1 is a gene essential for mental health, and mutations in the gene lead to severe intellectual disability, epilepsy, and autism. SYNGAP1 has several transcriptional start sites and multiple alternatively spliced exons (FIG. 1) producing at least 7 isoforms (Li et al., J. Biol. Chem., 276:21417-21424 (2001); and McMahon et al., Nat. Commun., 3:900 (2012)). Alternative splicing at the 5′ and 3′ ends of exon 18 generates at least 5 distinct SynGAP1 polypeptide isoforms (e.g., a SynGAP1-α1 polypeptide, a SynGAP1-α2 polypeptide, a SynGAP1-α3 polypeptide, a SynGAP1-β polypeptide, and a SynGAP1-γ polypeptide), each of which possesses different protein binding motifs and differentially regulates synaptic function (McMahon et al., Nat. Commun., 3:900 (2012); and Araki et al., eLife, 9: e56273 (2020)).

SYNGAP1-related Intellectual Disability (SRID, MRD5) is a severe neurodevelopmental disorder (NDD) characterized by intellectual disability (ID), autism spectrum disorder (ASD), and epilepsy (Vlaskamp et al., Neurology, 92(2): e96-e107 (2019); and Jimenez-Gomez et al., J. Neurodev. Disord., 11(1): 18 (2019)). SRID is estimated to account for 0.5-1% of all NDD and ˜1% of the ˜200 million ID cases worldwide (UK-DDD-study, Nature, 519(7542):223-8 (2015); Carvill et al., Nat. Genet., 45(7):825-30 (2013); Berryer et al., Hum. Mutat., 34(2): 385-94 (2013); Hamdan et al., Am. J. Hum. Genet., 88(3): 306-16 (2011); Hamdan et al., N. Engl. J. Med., 360 (6): 599-605 (2009); and Lopez-Rivera et al., Brain, 143 (4): 1099-105 (2020)) with no disease modifying treatment available.

### SUMMARY

SynGAP1-α1 polypeptide has a major role in synapse function (e.g., as compared to SynGAP1-α2 polypeptides, SynGAP1-β polypeptides, and SynGAP1-γ polypeptides). This disclosure is based, at least in part, on the development of splice reporter nucleic acid constructs (e.g., splice reporter plasmids) that allow the rapid screening of agents (e.g., ASOs) that can reduce or eliminate expression of SynGAP1-α2, SynGAP1-α3, SynGAP1-B, and SynGAP1-γ polypeptide isoforms and/or can increase expression of a SynGAP1-α1 polypeptide isoform and/or a SynGAP1-α2 polypeptide isoform, and the discovery that ASOs that can be used to modulate SYNGAP1 splicing (e.g., SSOs) to increase expression of a SynGAP1-α1 polypeptide and/or a SynGAP1-α2 polypeptide in a mammal (e.g., a human) having, or at risk of developing, a SYNGAP1-associated neurodevelopmental disorders (NDD) to treat the mammal.

This document provides splice reporter nucleic acid constructs (e.g., splice reporter plasmids). For example, this document provides splice reporter plasmids that can (e.g., are designed to) identify which isoform(s) of a SynGAP1 polypeptide are being expressed by a cell (e.g., a neuron) and can therefore be used to screen for agents (e.g., ASOs) that can modulate SYNGAP1 splicing. In some cases, a splice reporter nucleic acid construct (e.g., splice reporter plasmid) can include (a) a promoter sequence operably linked to a nucleic acid encoding a first reporter polypeptide; (b) a SYNGAP1 minigene comprising (i) two or more exons and the intervening intron(s), (ii) a first splice donor/acceptor pair, and (iii) a second splice donor/acceptor pair; (c) nucleic acid encoding a second reporter polypeptide in frame with the first splice donor/acceptor pair; and (d) nucleic acid encoding a third reporter polypeptide in frame with the second splice donor/acceptor pair; where the first reporter polypeptide, the second reporter polypeptide, and the third reporter polypeptide are each a different reporter polypeptide. For example, a splice reporter nucleic acid construct (e.g., splice reporter plasmid) can be a chromatic reporter that can include a promoter sequence operably linked to a nucleic acid encoding a first reporter polypeptide (e.g., a TagBFP2 polypeptide), a SYNGAP1 minigene (e.g., a SYNGAP1 c-terminal minigene), a nucleic acid encoding a second reporter polypeptide (e.g., an eGFP polypeptide) in a −1 reading frame relative to the SYNGAP1 minigene and lacking a stop codon, and a nucleic acid encoding a third reporter polypeptide (e.g., a mCherry polypeptide) in a +0 reading frame, where a first splice event results in expression of a fusion polypeptide including the first reporter polypeptide, a fragment of a first SynGAP1 polypeptide isoform, and the second reporter polypeptide, and where a second splice event results in expression of a fusion polypeptide including the first reporter polypeptide, a fragment of a second SynGAP1 polypeptide isoform, and the third reporter polypeptide. Such a construct is also referred to herein as a trichromatic splice reporter nucleic acid construct. In some cases, a splice reporter nucleic acid construct and a candidate splice modulating agent (e.g., a candidate SSO) can be delivered to a cell, and a splice event can be rapidly determined based, at least in part, on which reporter polypeptides are detected.

This document also provides methods and materials for modulating SYNGAP1 splicing. For example, one or more agents that can modulate SYNGAP1 splicing (e.g., one or more SSOs) can be delivered to a cell to reduce or eliminate expression of a SynGAP1-α2 polypeptide, a SynGAP1-α3 polypeptide, a SynGAP1-β polypeptide, and/or a SynGAP1-γ polypeptide within that cell. For example, one or more agents that can modulate SYNGAP1 splicing (e.g., one or more SSOs) can be delivered to a cell to increase expression of a SynGAP1-α1 polypeptide and/or a SynGAP1-α2 polypeptide within that cell. For example, one or more agents that can modulate SYNGAP1 splicing (e.g., one or more SSOs) can be administered to a mammal (e.g., a human) having, or at risk of developing a SYNGAP1-associated NDD, to reduce or eliminate expression of a SynGAP1-α2 polypeptide, a SynGAP1-α3 polypeptide, a SynGAP1-β polypeptide, and/or a SynGAP1-γ polypeptide in cells within that mammal (e.g., to treat the mammal). For example, one or more agents that can modulate SYNGAP1 splicing (e.g., one or more SSOs) can be administered to a mammal (e.g., a human) having, or at risk of developing a SYNGAP1-associated NDD, to increase expression of a SynGAP1-α1 polypeptide and/or a SynGAP1-α2 polypeptide in cells within that mammal (e.g., to treat the mammal).

As demonstrated herein, splice reporter nucleic acid constructs (e.g., splice reporter plasmids) described herein can be used to distinguish which isoform of a SynGAP1 polypeptide is being expressed by a cell, and can be used to screen for agents (e.g., ASOs) that can modulate SYNGAP1 splicing (e.g., SSOs). Also as demonstrated herein, one or more agents that can modulate SYNGAP1 splicing (e.g., one or more SSOs) can be used to suppress (e.g., reduce or eliminate) expression of a SynGAP1-α2 polypeptide, a SynGAP1-α3 polypeptide, a SynGAP1-β polypeptide, and/or a SynGAP1-γ polypeptide, and can be used to increase expression of a SynGAP1-α1 polypeptide and/or a SynGAP1-α2 polypeptide.

Having the ability to modulate SYNGAP1 splicing using one or more SSOs described herein can allow one to reduce or eliminate expression of one or more non-functional isoforms of a SynGAP1 polypeptide (e.g., SynGAP1-α2 polypeptide, a SynGAP1-α3 polypeptide, a SynGAP1-β polypeptide, and/or a SynGAP1-γ polypeptide), and can increase expression of a functional isoform of a SynGAP1 polypeptide (e.g., a SynGAP1-α1 polypeptide and/or a SynGAP1-α2 polypeptide) providing a unique and unrealized opportunity to treat SYNGAP1-associated NDDs such as intellectual disability, autism, epilepsy, and schizophrenia. Further, the splice reporter nucleic acid constructs (e.g., splice reporter plasmids) described herein can be used to easily and efficiently screen additional candidate agents to identify additional agents having the ability to modulate SYNGAP1 splicing. Splice reporter nucleic acid constructs provided herein also can be used to characterize the highly non-canonical splicing event at the 3′ end of the SYNGAP1 gene.

In general, one aspect of this document features nucleic acid constructs for identifying a SYNGAP1 splice event. Such a nucleic acid construct can include (a) a promoter sequence operably linked to a nucleic acid encoding a first reporter polypeptide; (b) a SYNGAP1 minigene comprising (i) at least two exons and intervening intron(s), (ii) a first splice donor/acceptor pair, and (iii) a second splice donor/acceptor pair; (c) nucleic acid encoding a second reporter polypeptide in frame with the first splice donor/acceptor pair; and (d) nucleic acid encoding a third reporter polypeptide in frame with the second splice donor/acceptor pair; where the first reporter polypeptide, the second reporter polypeptide, and the third reporter polypeptide are each a different reporter polypeptide. The nucleic acid construct can be a plasmid. The promoter can be a CAG promoter, a tTA promoter, a CaMKII promoter, or a Syn1 promoter. The first splice donor/acceptor pair can be a canonical splice donor/acceptor pair. The second splice donor/acceptor pair can be a non-canonical splice donor/acceptor pair. The first reporter polypeptide, the second reporter polypeptide, and the third reporter polypeptide can each be a fluorescent polypeptide. The first reporter polypeptide, the second reporter polypeptide, and the third reporter polypeptide can each independently be a blue fluorescent polypeptide, a green fluorescent polypeptide, a mCherry polypeptide, a emiRFP670 polypeptide, a firefly luciferase polypeptide, or a Renilla luciferase polypeptide. The SYNGAP1 minigene can include exons 17 and 18 and the intervening intron. The SYNGAP1 minigene can include a sequence set forth in SEQ ID NO: 1. The SYNGAP1 minigene can include exons 10 and 11 and the intervening intron. The SYNGAP1 minigene can include a sequence set forth in SEQ ID NO:2. The SYNGAP1 minigene can include exons 18, 19, and 20, and the intervening introns. The SYNGAP1 minigene can include a sequence set forth in SEQ ID NO:3. The nucleic acid construct also can include a nucleic acid encoding a fourth reporter polypeptide in frame with a third splice donor/acceptor pair.

In another aspect, this document features methods for identifying a splice-switching SSO that can modulate SYNGAP1 gene splicing. The methods can include, or consist essentially of, (a) delivering a candidate SSO to a cell; (b) delivering the nucleic acid construct for identifying a SYNGAP1 splice event to the cell; and (c) detecting the first reporter polypeptide, the second reporter polypeptide, and the third reporter polypeptide, where a SSO that modulates SYNGAP1 splicing at the first splice donor/acceptor pair is identified when the first reporter polypeptide and the second reporter polypeptide are detected; and where a SSO that modulates SYNGAP1 splicing at the second splice donor/acceptor pair is identified when the first reporter polypeptide and the third reporter polypeptide are detected. The nucleic acid construct can include (a) a promoter sequence operably linked to a nucleic acid encoding a first reporter polypeptide; (b) a SYNGAP1 minigene comprising (i) at least two exons and intervening intron(s), (ii) a first splice donor/acceptor pair, and (iii) a second splice donor/acceptor pair; (c) nucleic acid encoding a second reporter polypeptide in frame with the first splice donor/acceptor pair; and (d) nucleic acid encoding a third reporter polypeptide in frame with the second splice donor/acceptor pair; where the first reporter polypeptide, the second reporter polypeptide, and the third reporter polypeptide are each a different reporter polypeptide. The nucleic acid construct can be a plasmid. The promoter can be a CAG promoter, a tTA promoter, a CaMKII promoter, or a Syn1 promoter. The first splice donor/acceptor pair can be a canonical splice donor/acceptor pair. The second splice donor/acceptor pair can be a non-canonical splice donor/acceptor pair. The first reporter polypeptide, the second reporter polypeptide, and the third reporter polypeptide can each be a fluorescent polypeptide. The first reporter polypeptide, the second reporter polypeptide, and the third reporter polypeptide can each independently be a blue fluorescent polypeptide, a green fluorescent polypeptide, a mCherry polypeptide, a emiRFP670 polypeptide, a firefly luciferase polypeptide, or a Renilla luciferase polypeptide. The SYNGAP1 minigene can include exons 17 and 18 and the intervening intron. The SYNGAP1 minigene can include a sequence set forth in SEQ ID NO: 1. The SYNGAP1 minigene can include exons 10 and 11 and the intervening intron. The SYNGAP1 minigene can include a sequence set forth in SEQ ID NO:2. The SYNGAP1 minigene can include exons 18, 19, and 20, and the intervening introns. The SYNGAP1 minigene can include a sequence set forth in SEQ ID NO:3. The nucleic acid construct also can include a nucleic acid encoding a fourth reporter polypeptide in frame with a third splice donor/acceptor pair.

In another aspect, this document features methods for modulating SYNGAP1 gene splicing in a cell. The methods can include, or consist essentially of, administering to a cell an SSO that can reduce or eliminate expression of a SynGAP1-α2 polypeptide, a SynGAP1-α3 polypeptide, a SynGAP1-β polypeptide, or a SynGAP1-γ polypeptide, where the SSO targets a splice site within a SYNGAP1 gene. The splice site can be a splice donor site, a splice acceptor site, an exonic splice enhancer, an exonic splice silencer, an intronic splice enhancer, or an intronic splice silencer. The splice site can include the nucleic acid sequence GCTCAGgtggaa (SEQ ID NO:5). The splice site can include the nucleic acid sequence ttgcagGAGAGG (SEQ ID NO:7). The SSO can include a nucleic acid sequence set forth in any one of SEQ ID NOs: 8-39 or 71-114.

In another aspect, this document features methods for increasing a level of a SynGAP1-α1 polypeptide or a SynGAP1-α2 polypeptide in a cell. The methods can include, or consist essentially of, administering to a cell an SSO that targets a splice site within a SYNGAP1 gene. The splice site can be a splice donor site, a splice acceptor site, an exonic splice enhancer, an exonic splice silencer, an intronic splice enhancer, or an intronic splice silencer. The splice site can include the nucleic acid sequence GCTCAGgtggaa (SEQ ID NO: 5). The splice site can include the nucleic acid sequence ttgcagGAGAGG (SEQ ID NO: 7). The SSO can include a nucleic acid sequence set forth in any one of SEQ ID NOs: 8-39 or 71-114.

In another aspect, this document features methods for treating a mammal having or at risk of developing a SYNGAP1-associated NDD. The methods can include, or consist essentially of, administering to a mammal having or at risk of developing a SYNGAP1-associated NDD an SSO that targets a splice site within a SYNGAP1 gene. The mammal can be a human. The human can be an infant (e.g., a newborn). The SYNGAP1-associated NDD can be a SRID, SYNGAP1-related ASD, SYNGAP1-related epilepsy, sleep disorders, intellectual disability, or schizophrenia. The administering can be an intracerebroventricular (ICV) injection, an intracerebral injection, a retroorbital injection, an intravenous injection, a sinus injection, or an intrathecal injection. The splice site can be a splice donor site, a splice acceptor site, an exonic splice enhancer, an exonic splice silencer, an intronic splice enhancer, or an intronic splice silencer. The splice site can include the nucleic acid sequence GCTCAGgtggaa (SEQ ID NO:5). The splice site can include the nucleic acid sequence ttgcagGAGAGG (SEQ ID NO:7). The SSO can include a nucleic acid sequence set forth in any one of SEQ ID NOs: 8-39 or 71-114.

In another aspect, this document features uses of an SSO that targets a splice site within a SYNGAP1 gene to treat a mammal having a SYNGAP1-associated NDD.

In another aspect, this document features SSOs that targets a splice site within a SYNGAP1 gene for use in the preparation of a medicament for treating a mammal having a SYNGAP1-associated NDD.

In another aspect, this document features SSOs that targets a splice site within a SYNGAP1 gene for use in the treatment of a mammal having a SYNGAP1-associated NDD.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1. Gene and exon structure of SYNGAP1, annotated with corresponding alternative splice sites and isoforms. Two introns display inefficient splicing and are often retained, preventing mRNA maturation and expression. C-terminal splicing at exon 18 and 19 is expanded at the bottom.

FIGS. 2A-2D. SYNGAP1 α1 arises from a non-canonical splicing event unique to brain cells and conserved across many mammalian species. FIG. 2A) Three c-terminal isoforms arising from exon 18-exon 20 splice junctions. A canonical (normal) splice donor sequence (SEQ ID NO:51) and splice acceptor sequence (SEQ ID NO:52) are shown. Also shown are an α2 splice junction (SEQ ID NO:53), an α1 splice junction (SEQ ID NO:54), and an α3 splice junction (SEQ ID NO:55). α1 and α3 are non-canonical, and α3 is novel. FIG. 2A bottom right) No non-canonical splicing was observed outside of the brain. α1 non-canonical splicing is observed across multiple mammalian species where brain RNA-seq data is available. FIGS. 2B and 2C) Composition of SYNGAP1 exon 18-exon 20 splice isoforms across human tissues (FIG. 2B) and mouse tissues (FIG. 2C) from published RNA-seq data. *: No reads were mapped to the target region in heart across all replicates. FIG. 2D) Alignment of exon 18 and adjacent sequences from several mammalian species are shown, from top to bottom are SEQ ID NOs: 56-66.

FIGS. 3A-3D. Trichromatic splice reporter construct for SYNGAP1 c-terminal alternative splicing. FIG. 3A) Schematic of SYNGAP1 splice reporter construct that fluorescently reports relative expression of SYNGAP-α1 (eGFP) and α2 (mCherry) through the frameshift arising from alternative splicing. FIG. 3B) Confocal images of primary cultured rat hippocampal neurons that express the trichromatic splice reporter construct. Total SYNGAP1 minigene expression is reported by mTagBFP2 expression which is in all minigene transcripts. A deletion of ˜800 bp in intron 18 (Δ1) abrogated eGFP expression. FIG. 3C) The blue, green and red fluorescence values of each cell, when plotted in 3D space, fall on a single plane, as expected by design. FIG. 3D) Using coefficients estimated from (FIG. 3C), the normalized ratio of eGFP/TagBFP2 can be plotted as an estimate of the portion of α1-splicing in this minigene reporter. The results show the Δ1 intronic region is necessary for α1 non-canonical splicing. Note that this ratio is only an estimate of the ratio of non-canonical splicing, due to other possible minor splice events in reporter. FIG. 3E) The normalized (eGFP+mCherry)/TagBFP2 values are unchanged with the intronic deletions, including D2 which includes the gamma exon. The ΔAll deletion, spanning from intron region 1 to 5, leads to the activation of a cryptic splice donor that shifts the reading frame of the spliced transcript, resulting in eGFP expression under α2 splicing conditions rather than α1.

FIGS. 4A-4C. A trichromatic splice reporter reveals SYNGAP1 c-terminal non-canonical alternative splicing depends on intronic region Δ1A (+13-+244). FIG. 4A) Schematic of SYNGAP1 splice reporter construct and deletions. FIG. 4B) Large deletions of Δ1-4 and D2-5 confirm that intronic region Δ1 is necessary for α1 non-canonical splicing. FIG. 4C) Small deletions of Δ1A reveal that the intronic region Δ(+13-+244) is necessary for α1 non-canonical splicing.

FIGS. 5A-5D. SYNGAP1 c-terminal α1 non-canonical splicing requires a conserved intronic region Δ1A-conserved (+13-+54). FIG. 5A) Schematic of SYNGAP1 splice reporter construct and deletions. FIGS. 5B-5D) Small deletions of Δ1A-conserved and Δ1A-non-conserved reveal that the conserved intronic region is necessary for α1 non-canonical splicing. Δ1BC, Δ2-5 and ΔExon20 deletions increase α1:(α1+α2) ratio, suggesting a suppressive effect on α1 non-canonical splicing. Δ1A and Δ2-5 constructs display a drop in (mCherry+eGFP)/TagBFP2, suggesting an increase in cryptic splicing or γ (exon 19) splicing (exon 19 inclusion).

FIGS. 6A-6E. Point mutations in the SYNGAP1 splice reporter abolish α1 splicing. FIG. 6A) Schematic of SYNGAP1 splice reporter construct and mutations/deletions. FIGS. 6B-6D) Several point mutations and their impact on splice reporter fluorescence ratios. ΔPal1 and ΔPal2 mutate key bases in palindromic repeats within Exon 18, and ΔPal2 specifically abolishes eGFP expression (α1 splicing). The GTA (Intron 18+3G>A) mutation also prevents eGFP expression, while the AGT (Exon 20±1G>T) mutation does not. A large deletion coupled with the mutation of a cryptic splice donor Δ1-5A>T results in very little eGFP expression, as expected by the inclusion of the Δ1 region in the deletion. Note the y-axis in (FIG. 6B) and (FIG. 6C) use TagBFP2 FL as the denominator to accommodate a construct (AGT) where the mutation introduces a stop codon in the mCherry reading frame. FIG. 6E) Amplicon sequencing of cDNA from the control splice reporter and Δ1A, Δ1-5A>T reveal that the initial conserved intronic region is necessary for α1 and α3 non-canonical splicing. ΔPal2 mutation abolishes α1 in favor for α3, while GTA mutation decimates both. The AGT mutation preferentially suppresses α3 splicing.

FIGS. 7A-7H. Trichromatic splice reporter construct for SYNGAP1 β/non-β alternative splicing. FIG. 7A) Schematic of SYNGAP1 splice reporter construct that fluorescently reports relative expression of SYNGAP1 β (mCherry) and non-β (eGFP) and through the frameshift arising from alternative splicing. FIGS. 7B-7C) FL ratios from primary cultured rat hippocampal cells that are transfected with the trichromatic splice reporter construct. Aspiny neurons display lower mCherry and higher eGFP ratios compared to spiny pyramidal neurons and glial cells. FIG. 7D) The values of (mCherry+eGFP)/TagBFP2 are lower than unity, suggesting the existence of splicing outcomes other than the expected β and non-β or unstable protein products. FIG. 7E) Published single-cell RNA-seq data shows lower β usage in PV+interneurons in comparison to (spiny) pyramidal neurons and astrocytes (glial), consistent with the results from the β splice reporter (FIGS. 7B and 7C). Sequences shown are SEQ ID NOs: 67 and 68. FIGS. 7F-7H) A splice acceptor mutation in the β acceptor leads to abolished mCherry expression, demonstrating the ability of the reporter to reflect splice changes in fluorescence levels.

FIGS. 8A-8D. Titration of ASOs and co-transfected SYNGAP1 trichromatic splice reporter. FIG. 8A-8C) A negative control ASO (NC5) and two SYNGAP1 ASOs (A2/A3) were co-transfected using Lipofectamine with the SYNGAP1 trichromatic splice reporter (SGR; 1 μg unless noted otherwise) at various doses (pmol). The ratios of FL remained stable across 3-150 pmol of ASO treatment. FIG. 8D) The FL expression was low at higher doses of ASO, and 150 pmol of ASO treatment led to a single neuron transfected, demonstrating a decrease of plasmid transfection rate at high doses of ASOs.

FIGS. 9A-9D. Screen for SYNGAP1 SSOs using a trichromatic splice reporter. FIG. 9A) 2 μg of splice reporter plasmid was co-transfected together with 15 pmol of each ASO to screen for impact on SYNGAP1 c-terminal splicing. The mCherry/(mCherry+eGFP) level, indicative of the portion of α2 splicing, was only increased in the Δ1A group. FIG. 9B) The eGFP/(mCherry+eGFP) level, indicative of the portion of α1 splicing, was decimated in the Δ1A group but relatively stable in other groups. FIG. 9C) The (mCherry+eGFP)/TagBFP2 values were significantly increased with the treatment of ASOs Exon 18+84 and Intron 18+20. FIG. 9D) Schematic of SYNGAP1 splice reporter construct and ASO target areas.

FIGS. 10A-10G. Systematic deletions revealed an evolutionarily conserved region necessary for α1 splicing. FIG. 10A) Schematic representation of the reporter regions tested in the systematic deletion approach. FIGS. 10B-10C) Quantification of normalized eGFP/(eGFP+mCherry) (FIG. 10B) and normalized (eGFP+mCherry)/TagBFP2 ratios (FIG. 10C) based on confocal microscopy. FIG. 10D) ratios of reads corresponding to each isoform based on amplicon sequencing data from neurons transfected with respective reporter constructs. FIGS. 10E-10F) Quantification of normalized eGFP/(eGFP+mCherry) (FIG. 10E) and normalized (eGFP+mCherry)/TagBFP2 ratios (FIG. 10F) based on confocal microscopy. FIG. 10G) ratios of reads corresponding to each isoform based on amplicon sequencing data from neurons transfected with respective reporter constructs.

FIGS. 11A-11D. Specific deletions and mutations highlighted the role of one nucleotide instead of a predicted RNA secondary structure. FIG. 11A) Schematic representation of the region of focus (SEQ ID NO:191) on the trichromatic reporter. FIGS. 11B-11C) Quantification of normalized eGFP/(eGFP+mCherry) (FIG. 11B) and normalized (eGFP+mCherry)/TagBFP2 ratios (FIG. 11C) based on confocal microscopy. FIG. 11D) Ratios of reads corresponding to each isoform based on amplicon sequencing data from neurons transfected with the respective reporter constructs.

FIGS. 12A-12C. Manipulations of the splice donor site disrupted α1 splicing and induced the use of an alternative donor site. FIG. 12A) Schematic representation of the mutations (SEQ ID NOs: 192-199, numbered from top to bottom) tested near the splice donor site (SEQ ID NO:191). FIG. 12B) Quantification of ratios of reads corresponding to each isoform based on amplicon sequencing data from neurons transfected with respective reporter construct. FIG. 12C) Schematic representation of the splice variants commonly observed (top; SEQ ID NO:200) and generated after manipulating the original splice donor site (bottom; SEQ ID NO:201).

FIGS. 13A-13D. Large deletions disrupted α1 splicing and triggered cryptic splicing. FIG. 13A) Schematic representation of the large deletions, the cryptic acceptor and donor, and associated mutations to correct for cryptic splicing. FIGS. 13B-13C) Quantification of normalized eGFP/(eGFP+mCherry) (FIG. 13B) and normalized (eGFP+mCherry)/TagBFP2 ratios (FIG. 13C) based on confocal microscopy. FIG. 13D) Ratios of reads corresponding to each isoform based on amplicon sequencing data from neurons transfected with respective reporter constructs.

FIGS. 14A-14D. A systematic ASO walk identified α1-regulating ASOs with therapeutic potential. FIG. 14A) Schematic representation of the ASOs screened, their target site (SEQ ID NO:202), and their corresponding effects. Red: decreases α1 splicing. Green: increases α1 splicing. FIGS. 14B-14C) Quantification of normalized eGFP/(eGFP+mCherry) (FIG. 14B) and normalized (eGFP+mCherry)/TagBFP2 ratios (FIG. 14C) based on confocal microscopy. FIG. 14D) ratios of reads corresponding to each isoform based on amplicon sequencing data from neurons transfected with respective reporter constructs.

FIGS. 15A-15B. One candidate ASO mediated a dose-dependent increase of α1 splicing. FIG. 15A) Quantification of normalized eGFP/(eGFP+mCherry) based on confocal microscopy. FIG. 15B) Quantification of ratios of reads corresponding to each isoform based on amplicon sequencing data from neurons co-transfected with the wildtype reporter and respective ASOs.

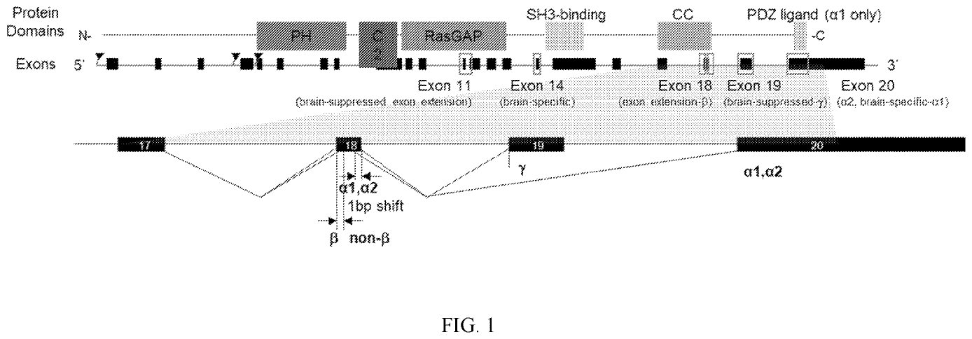

### DETAILED DESCRIPTION

This document provides splice reporter nucleic acid constructs (e.g., splice reporter plasmids). For example, this document provides splice reporter plasmids that can (e.g., are designed to) identify which isoform(s) of a SynGAP1 polypeptide are being expressed by a cell (e.g., a neuron). Splice reporter plasmids provided herein (e.g., splice reporter plasmids that can identify which isoform(s) of a SynGAP1 polypeptide are being expressed by a cell such as a neuron) can be used to screen for agents (e.g., ASOs) that can modulate SYNGAP1 splicing. In some cases, a splice reporter nucleic acid construct (e.g., splice reporter plasmid) can include (a) a promoter sequence operably linked to a nucleic acid encoding a first reporter polypeptide; (b) a SYNGAP1 minigene comprising (i) two or more exons and the intervening intron(s), (ii) a first splice donor/acceptor pair, and (iii) a second splice donor/acceptor pair; (c) nucleic acid encoding a second reporter polypeptide in frame with the first splice donor/acceptor pair; and (d) nucleic acid encoding a third reporter polypeptide in frame with the second splice donor/acceptor pair; where the first reporter polypeptide, the second reporter polypeptide, and the third reporter polypeptide are each a different reporter polypeptide. For example, a splice reporter nucleic acid construct (e.g., splice reporter plasmid) can include a promoter sequence operably linked to a nucleic acid encoding a first reporter polypeptide, a SYNGAP1 minigene (e.g., a SYNGAP1 c-terminal minigene), a nucleic acid encoding a second reporter polypeptide in a −1 reading frame relative to the SYNGAP1 minigene and lacking a stop codon, and a nucleic acid encoding a third reporter polypeptide, where a first splice event results in expression of a fusion polypeptide including the first reporter polypeptide, a fragment of a first SynGAP1 polypeptide isoform, and the second reporter polypeptide, and where a second splice event results in expression of a fusion polypeptide including the first reporter polypeptide, a fragment of a second SynGAP1 polypeptide isoform, and the third reporter polypeptide.

A splice reporter nucleic acid construct provided herein (e.g., a splice reporter plasmid that can identify which isoform(s) of a SynGAP1 polypeptide are being expressed by a cell such as a neuron) can include any appropriate SYNGAP1 minigene. In some cases, a splice reporter nucleic acid construct provided herein can include a SYNGAP1 c-terminal minigene. As used herein, a “SYNGAP1 minigene” can be any fragment of an endogenous SYNGAP1 gene that includes two or more contiguous exons and the intervening intron(s) which contain two or more splice donor/acceptor pairs, such that each donor/acceptor pair present in the SYNGAP1 minigene can produce a different mRNA which can, in turn, each encode a fragment of a distinct SynGAP1 polypeptide isoform. For example, a SYNGAP1 minigene can include two or more contiguous exons and the intervening intron(s) which contain two or more alternative splice sites such that a single SYNGAP1 minigene can express two or more SynGAP1 polypeptide isoforms. In some cases, a SYNGAP1 minigene that can be included in a splice reporter nucleic acid construct provided herein can include two exons and the intervening intron and can contain two or more alternative splice sites. For example, a SYNGAP1 minigene that can be included in a splice reporter nucleic acid construct provided herein can include exons 17 and 18 and the intervening intron. For example, a SYNGAP1 minigene that can be included in a splice reporter nucleic acid construct provided herein can include the nucleic acid sequence set forth in SEQ ID NO:1 (see, e.g., Example 3). For example, a SYNGAP1 minigene that can be included in a splice reporter nucleic acid construct provided herein can include exons 10 and 11 and the intervening intron. For example, a SYNGAP1 minigene that can be included in a splice reporter nucleic acid construct provided herein can include the nucleic acid sequence set forth in SEQ ID NO:2 (see, e.g., Example 3). In some cases, a SYNGAP1 minigene that can be included in a splice reporter nucleic acid construct provided herein can include three exons and the intervening introns and can contain two or more alternative splice sites. For example, a SYNGAP1 minigene that can be included in a splice reporter nucleic acid construct provided herein can include exons 18, 19, and 20 and the intervening introns. For example, a SYNGAP1 minigene that can be included in a splice reporter nucleic acid construct provided herein can include the nucleic acid sequence set forth in SEQ ID NO:3 (see, e.g., Example 3).

A splice reporter nucleic acid construct provided herein (e.g., a splice reporter plasmid that can identify which isoform(s) of a SynGAP1 polypeptide are being expressed by a cell such as a neuron) can include nucleic acid encoding any appropriate reporter polypeptide. In some cases, each reporter polypeptide encoded by nucleic acid present in a splice reporter nucleic acid construct provided herein is different. In some cases, a reporter polypeptide encoded by nucleic acid present in a splice reporter nucleic acid construct provided herein can be fluorescent polypeptide. Examples of nucleic acid encoding a fluorescent polypeptide that can be included in a splice reporter nucleic acid construct provided herein include, without limitation, nucleic acid encoding a blue fluorescent polypeptide (BFP; e.g., nucleic acid encoding a constitutive BFP such as TagBFP2), nucleic acid encoding a green fluorescent polypeptide (GFP; e.g., nucleic acid encoding an enhanced GFP (eGFP), nucleic acid encoding a mCherry polypeptide, nucleic acid encoding a emiRFP670 polypeptide, and luciferases (e.g., firefly luciferases and Renilla luciferases). In cases where a splice reporter nucleic acid construct provided herein includes nucleic acid encoding one or more fluorescent polypeptides, the construct can be referred to as a chromatic splice reporter nucleic acid construct. In cases where a splice reporter nucleic acid construct provided herein includes nucleic acid encoding one or more enzymatic polypeptides, the construct can be referred to as as enzymatic splice reporter nucleic acid construct.

A splice reporter nucleic acid construct provided herein (e.g., a splice reporter plasmid that can identify which isoform(s) of a SynGAP1 polypeptide are being expressed by a cell such as a neuron) can include any appropriate promoter sequence. A promoter can be a naturally occurring promoter or a recombinant promoter. A promoter can be a constitutive promoter or an inducible promoter. A promoter can be a ubiquitous promoter or a tissue/cell-specific promoter (e.g., a neuron-specific promoter). Examples of promoters that can be used to drive expression of a fusion polypeptide (e.g., a fusion polypeptide including a first reporter polypeptide, a fragment of a SynGAP1 polypeptide isoform, and a second reporter polypeptide) from a splice reporter nucleic acid construct provided herein include, without limitation, CAG promoters, tTA promoters, CaMKII promoters, and Syn1 promoters. As used herein, “operably linked” refers to positioning of a promoter within a splice reporter nucleic acid construct provided herein in such a way as to permit or facilitate expression of a fusion polypeptide including a first reporter polypeptide (e.g., a fusion polypeptide including a first reporter polypeptide, a fragment of a first SynGAP1 polypeptide isoform, and a second reporter polypeptide or a fusion polypeptide including a first reporter polypeptide, a fragment of a second SynGAP1 polypeptide isoform, and a third reporter polypeptide).

In some cases, a splice reporter nucleic acid construct provided herein can include (a) a CAG promoter sequence operably linked to a nucleic acid encoding a BFP polypeptide (e.g., a TagBFP2 polypeptide), (b) a SYNGAP1 minigene including exons 18, 19, and 20 and the intervening introns, (c) a nucleic acid encoding a GFP polypeptide (e.g., an eGFP polypeptide) in a −1 reading frame relative to the SYNGAP1 minigene and lacking a stop codon, and (d) a nucleic acid encoding a mCherry polypeptide in a +0 reading frame. A first splice event can result in expression of a fusion polypeptide including a BFP polypeptide (e.g., a TagBFP2 polypeptide), a fragment of a first SynGAP1 polypeptide isoform, and a GFP polypeptide (e.g., an eGFP polypeptide). A second splice event results in expression of a fusion polypeptide including a BFP polypeptide (e.g., a TagBFP2 polypeptide), a fragment of a second SynGAP1 polypeptide isoform, and a mCherry polypeptide.

In some cases, a splice reporter nucleic acid construct provided herein can include (a) a CAG promoter sequence operably linked to a nucleic acid encoding a BFP polypeptide (e.g., a TagBFP2 polypeptide), (b) a SYNGAP1 minigene including exons 10 and 11 and the intervening introns, (c) a nucleic acid encoding a GFP polypeptide (e.g., an eGFP polypeptide) in a −1 reading frame relative to the SYNGAP1 minigene and lacking a stop codon, and (d) a nucleic acid encoding a mCherry polypeptide in a +0 reading frame. A first splice event can result in expression of a fusion polypeptide including a BFP polypeptide (e.g., a TagBFP2 polypeptide), a fragment of a first SynGAP1 polypeptide isoform, and a GFP polypeptide (e.g., an eGFP polypeptide). A second splice event results in expression of a fusion polypeptide including a BFP polypeptide (e.g., a TagBFP2 polypeptide), a fragment of a second SynGAP1 polypeptide isoform, and a mCherry polypeptide.

In some cases, a splice reporter nucleic acid construct provided herein can include (a) a CAG promoter sequence operably linked to a nucleic acid encoding a BFP polypeptide (e.g., a TagBFP2 polypeptide), (b) a SYNGAP1 minigene including exons 17 and 18 and the intervening introns, (c) a nucleic acid encoding a GFP polypeptide (e.g., an eGFP polypeptide) in a −1 reading frame relative to the SYNGAP1 minigene and lacking a stop codon, and (d) a nucleic acid encoding a mCherry polypeptide in a +0 reading frame. A first splice event can result in expression of a fusion polypeptide including a BFP polypeptide (e.g., a TagBFP2 polypeptide), a fragment of a first SynGAP1 polypeptide isoform, and a GFP polypeptide (e.g., an eGFP polypeptide). A second splice event results in expression of a fusion polypeptide including a BFP polypeptide (e.g., a TagBFP2 polypeptide), a fragment of a second SynGAP1 polypeptide isoform, and a mCherry polypeptide.

In some cases, a splice reporter nucleic acid construct provided herein can include (a) a CAG promoter sequence operably linked to a nucleic acid encoding a BFP polypeptide (e.g., a TagBFP2 polypeptide), (b) a SYNGAP1 minigene including exons 13, 14, and 15 and the intervening introns, (c) a nucleic acid encoding a GFP polypeptide (e.g., an eGFP polypeptide) in a −1 reading frame relative to the SYNGAP1 minigene and lacking a stop codon, and (d) a nucleic acid encoding a mCherry polypeptide in a +0 reading frame. A first splice event can result in expression of a fusion polypeptide including a BFP polypeptide (e.g., a TagBFP2 polypeptide), a fragment of a first SynGAP1 polypeptide isoform, and a GFP polypeptide (e.g., an eGFP polypeptide). A second splice event results in expression of a fusion polypeptide including a BFP polypeptide (e.g., a TagBFP2 polypeptide), a fragment of a second SynGAP1 polypeptide isoform, and a mCherry polypeptide.

This document also provides methods and materials for making and using splice reporter nucleic acid constructs provided herein (e.g., a splice reporter plasmid that can identify which isoform(s) of a SynGAP1 polypeptide are being expressed by a cell such as a neuron). In some cases, a splice reporter nucleic acid construct provided herein can be used to identify (e.g., to screen for) one or more agents that can modulate SYNGAP1 splicing. For example, a splice reporter nucleic acid construct and a candidate splice modulating agent (e.g., a candidate SSO) can be delivered to a cell, and a splice event can be determined based, at least in part, on which reporter polypeptides are detected. In some cases, a splice reporter nucleic acid construct including (a) a promoter sequence operably linked to a nucleic acid encoding a first reporter polypeptide, (b) a SYNGAP1 minigene (e.g., a SYNGAP1 c-terminal minigene), (c) a nucleic acid encoding a second reporter polypeptide in a −1 reading frame relative to the SYNGAP1 minigene and lacking a stop codon, and (d) a nucleic acid encoding a third reporter polypeptide in a +0 reading frame, and a candidate splice modulating agent (e.g., a candidate SSO) can be delivered to a cell, and detection of a fusion polypeptide including the first reporter polypeptide and the second reporter polypeptide can indicate that a first splice event resulting in expression of a first SynGAP1 polypeptide isoform has occurred. In some cases, a splice reporter nucleic acid construct including (a) a promoter sequence operably linked to a nucleic acid encoding a first reporter polypeptide, (b) a SYNGAP1 minigene (e.g., a SYNGAP1 c-terminal minigene), (c) a nucleic acid encoding a second reporter polypeptide in a −1 reading frame relative to the SYNGAP1 minigene and lacking a stop codon, and (d) a nucleic acid encoding a third reporter polypeptide in a +0 reading frame, and a candidate splice modulating agent (e.g., a candidate SSO) can be delivered to a cell, and detection of a fusion polypeptide including the first reporter polypeptide and the third reporter polypeptide can indicate that a second splice event resulting in expression of a second SynGAP1 polypeptide isoform has occurred. In some cases, an agent can be screened for the ability to modulate SYNGAP1 splicing as described in Example 1.

Any appropriate type of agent can be screened for the ability to modulate SYNGAP1 splicing. Examples of agents that can be screened for the ability to modulate SYNGAP1 splicing as described herein (e.g., using a splice reporter nucleic acid construct provided herein) include, without limitation, nucleic acid molecules (e.g., ASOs), small molecules, gene therapy vectors designed to alter splice factor expression, and targeted RNA-editing enzymes. In some cases, an agent that can be screened for the ability to modulate SYNGAP1 splicing as described herein can be a nucleic acid molecule that can target (e.g., can target and bind) a sequence present in a SYNGAP1 minigene included in a splice reporter nucleic acid construct provided herein.

In some cases, one or more agents that can modulate SYNGAP1 splicing (e.g., one or more SSOs) described herein can be formulated into a composition (e.g., a pharmaceutically acceptable composition) for administration to a mammal (e.g., a human) having, or at risk of developing a SYNGAP1-associated NDD. For example, a therapeutically effective amount of one or more agents that can modulate SYNGAP1 splicing (e.g., one or more SSOs) can be formulated together with one or more pharmaceutically acceptable carriers (additives) and/or diluents. A pharmaceutical composition can be formulated for administration in solid or liquid form including, without limitation, in the form of sterile solutions, suspensions, sustained-release formulations, tablets, capsules, pills, powders, or granules. Pharmaceutically acceptable carriers, fillers, and vehicles that can be used in a pharmaceutical composition described herein can include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

A pharmaceutical composition containing one or more agents that can modulate SYNGAP1 splicing (e.g., one or more SSOs) can be designed for oral or parenteral (including subcutaneous, intramuscular, intravenous intracerebral, retroorbital, sinus, intrathecal, and intradermal) administration. When being administered orally, a pharmaceutical composition can be in the form of a pill, tablet, or capsule. Compositions suitable for parenteral administration can include aqueous and non-aqueous sterile injection solutions that can contain anti-oxidants, buffers, bacteriostats, and solutes that render the formulation isotonic with the blood of the intended recipient. The formulations can be presented in unit-dose or multi-dose containers, for example, sealed ampules and vials, and can be stored in a freeze dried (lyophilized) condition requiring the addition of the sterile liquid carrier, for example, water for injections, immediately prior to use. Extemporaneous injection solutions and suspensions can be prepared from sterile powders, granules, and tablets.

This document also provides methods and materials for modulating SYNGAP1 splicing. For example, one or more agents that can modulate SYNGAP1 splicing (e.g., one or more SSOs) can be delivered to a cell to reduce or eliminate expression of a SynGAP1-α2 polypeptide, a SynGAP1-α3 polypeptide, a SynGAP1-B polypeptide, and/or a SynGAP1-γ polypeptide within that cell. For example, one or more agents that can modulate SYNGAP1 splicing (e.g., one or more SSOs) can be delivered to a cell to increase expression of a SynGAP1-α1 polypeptide and/or a SynGAP1-α2 polypeptide within that cell. For example, one or more agents that can modulate SYNGAP1 splicing (e.g., one or more SSOs) can be administered to a mammal (e.g., a human) having, or at risk of developing a SYNGAP1-associated NDD, to reduce or eliminate expression of a SynGAP1-α2 polypeptide, a SynGAP1-α3 polypeptide, a SynGAP1-β polypeptide, and/or a SynGAP1-γ polypeptide in cells within that mammal (e.g., to treat the mammal). For example, agents that can modulate SYNGAP1 splicing (e.g., one or more SSOs) can be administered to a mammal (e.g., a human) having, or at risk of developing a SYNGAP1-associated NDD, to increase expression of a SynGAP1-α1 polypeptide and/or a SynGAP1-α2 polypeptide in cells within that mammal (e.g., to treat the mammal).

Any appropriate agent that can modulate SYNGAP1 splicing (e.g., any appropriate SSO) can be delivered to a cell to modulate SYNGAP1 splicing within that cell. For example, an agent that can target (e.g., can target and bind) a splice site (e.g., a splice donor site or a splice acceptor site) within a SYNGAP1 gene can be delivered to a cell to modulate SYNGAP1 splicing within that cell. For example, an agent that can target (e.g., can target and bind) splice enhancer (e.g., an exonic splice enhancer (ESE) or an intronic splice enhancer (ISE)) within a SYNGAP1 gene can be delivered to a cell to modulate SYNGAP1 splicing within that cell. For example, an agent that can target (e.g., can target and bind) splice silencer (e.g., an exonic splice silencer (ESS) or an intronic splice silencer (ISS)) within a SYNGAP1 gene can be delivered to a cell to modulate SYNGAP1 splicing within that cell.

In some cases, an agent that can target (e.g., can target and bind) a splice site (e.g., a splice donor site or a splice acceptor site) within a SYNGAP1 gene to modulate SYNGAP1 splicing within a cell can target a canonical splicing donor/acceptor pair such that canonical SYNGAP1 splicing is inhibited and the expression of a SynGAP1-α2 polypeptide, a SynGAP1-α3 polypeptide, a SynGAP1-β polypeptide, and/or a SynGAP1-γ polypeptide is reduced or eliminated. For example, a SSO that can target a splice site comprising, consisting essentially of, or consisting of the nucleic acid sequence GCTCAGgtggaaattacaatgtcatttatcttctccgtgtcccatccccatccatcccac (SEQ ID NO:4) within a SYNGAP1 gene can modulate SYNGAP1 splicing to reduce or eliminate expression of a SynGAP1-α2 polypeptide, a SynGAP1-α3 polypeptide, a SynGAP1-β polypeptide, and/or a SynGAP1-γ polypeptide. In some cases, a SSO that can target a splice site comprising, consisting essentially of, or consisting of the nucleic acid sequence GCTCAGgtggaa (SEQ ID NO: 5) within a SYNGAP1 gene can modulate SYNGAP1 splicing to reduce or eliminate expression of a SynGAP1-α2 polypeptide, a SynGAP1-α3 polypeptide, a SynGAP1-β polypeptide, and/or a SynGAP1-γ polypeptide. For example, a SSO that can target a splice site comprising, consisting essentially of, or consisting of the nucleic acid sequence ttgcagGAGAGGCAGCTTCCCCCCTTGGGTCCAACAAACCCGC (SEQ ID NO:6) within a SYNGAP1 gene can modulate SYNGAP1 splicing to reduce or eliminate expression of a SynGAP1-α2 polypeptide, a SynGAP1-α3 polypeptide, a SynGAP1-β polypeptide, and/or a SynGAP1-γ polypeptide. In some cases, a SSO that can target a splice site comprising, consisting essentially of, or consisting of the nucleic acid sequence ttgcagGAGAGG (SEQ ID NO: 7) within a SYNGAP1 gene can modulate SYNGAP1 splicing to reduce or eliminate expression of a SynGAP1-α2 polypeptide, a SynGAP1-α3 polypeptide, a SynGAP1-β polypeptide, and/or a SynGAP1-γ polypeptide. In some cases, two or more agents that can modulate SYNGAP1 splicing (e.g., two or more SSOs) can be used. For example, a SSO that can target a splice site comprising, consisting essentially of, or consisting of the nucleic acid sequence GCTCAGgtggaaattacaatgtcatttatcttctccgtgtcccatccccatccatcccac (SEQ ID NO:4) within a SYNGAP1 gene and a SSO that can target a splice site comprising, consisting essentially of, or consisting of the nucleic acid sequence ttgcagGAGAGGCAGCTTCCCCCCTTGGGTCCAACAAACCCGC (SEQ ID NO:6) within a SYNGAP1 gene can modulate SYNGAP1 splicing to reduce or eliminate expression of a SynGAP1-α2 polypeptide, a SynGAP1-α3 polypeptide, a SynGAP1-β polypeptide, and/or a SynGAP1-γ polypeptide. For example, a SSO that can target a splice site comprising, consisting essentially of, or consisting of the nucleic acid sequence GCTCAGgtggaa (SEQ ID NO: 5) within a SYNGAP1 gene and a SSO that can target a splice site comprising, consisting essentially of, or consisting of the nucleic acid sequence ttgcagGAGAGG (SEQ ID NO: 7) within a SYNGAP1 gene can modulate SYNGAP1 splicing to reduce or eliminate expression of a SynGAP1-α2 polypeptide, a SynGAP1-α3 polypeptide, a SynGAP1-β polypeptide, and/or a SynGAP1-γ polypeptide. In some cases, a SSO that can target a splice site comprising, consisting essentially of, or consisting of the nucleic acid sequence cccactgcagCTCCTCATCAGGTAATT (SEQ ID NO:69) within a SYNGAP1 gene can modulate SYNGAP1 splicing to reduce or eliminate expression of a SynGAP1-α2 polypeptide, a SynGAP1-α3 polypeptide, a SynGAP1-β polypeptide, and/or a SynGAP1-Y polypeptide. In some cases, a SSO that can target a splice site comprising, consisting essentially of, or consisting of the nucleic acid sequence cccactgaagCCCGTCCCTT (SEQ ID NO: 70) within a SYNGAP1 gene can modulate SYNGAP1 splicing to reduce or eliminate expression of a SynGAP1-α2 polypeptide, a SynGAP1-α3 polypeptide, a SynGAP1-β polypeptide, and/or a SynGAP1-γ polypeptide. For example, a SSO that can target a splice site comprising, consisting essentially of, or consisting of the nucleic acid sequence cccactgcagCTCCTCATCAGGTAATT (SEQ ID NO:69) within a SYNGAP1 gene and a SSO that can target a splice site comprising, consisting essentially of, or consisting of the nucleic acid sequence cccactgaagCCCGTCCCTT (SEQ ID NO:70) within a SYNGAP1 gene can modulate SYNGAP1 splicing to reduce or eliminate expression of a SynGAP1-α2 polypeptide, a SynGAP1-α3 polypeptide, a SynGAP1-polypeptide, and/or a SynGAP1-γ polypeptide. For example, an agent (e.g., an SSO) that can reduce or eliminate expression of a SynGAP1-α2 polypeptide, a SynGAP1-α3 polypeptide, a SynGAP1-β polypeptide, and/or a SynGAP1-γ polypeptide can be used together with an agent (e.g., an SSO) that can increase expression of a SynGAP1-α1 polypeptide and/or a SynGAP1-α2 polypeptide. Examples of agents that can modulate SYNGAP1 splicing can be as shown in Table 1.

TABLE 1
Examples of agents that can modulate SYNGAP 1 splicing.

| SSO Start | SSO sequence | SEQ ID NO | target sequence | SEQ ID NO |
|---|---|---|---|---|
| SYNGAP1 E11 SA(−194) | 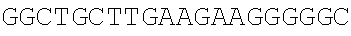 | 8 | GCCCCCTTCTTCAAGCAGCC | 115 |
| SYNGAP1 E11 SA(−184) | CAAGATGGGAGGCTGCTTGA | 9 | TCAAGCAGCCTCCCATCTTG | 116 |
| SYNGAP1 E11 SA(−174) | AGGAGCAAGATGGGAGGCTG | 10 | CAGCCTCCCATCTTGCTCCT | 117 |
| SYNGAP1 E11 SA(−169) | ACCGCAGGAGCAAGATGGGA | 11 | TCCCATCTTGCTCCTGCGGT | 118 |
| SYNGAP1 E11 SA(−13) | AGACCCTCAGCTTCCAGGGA | 12 | TCCCTGGAAGCTGAGGGTCT | 119 |
| SYNGAP1 E11 + 11 | AAACACCTCCTTCAGCTCCC | 13 | GGGAGCTGAAGGAGGTGTTT | 120 |
| SYNGAP1 E18 SA(−30) | 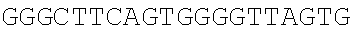 | 14 | CACTAACCCCACTGAAGCCC | 121 |
| SYNGAP1 E18 SA(−27) | 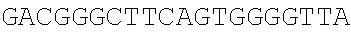 | 15 | TAACCCCACTGAAGCCCGTC | 122 |
| SYNGAP1 E18 SA(−20) | CTGAAGGGACGGGCTTCAGT | 16 | ACTGAAGCCCGTCCCTTCAG | 123 |
| SYNGAP1 E18 + 84 | TTGTAATTTCCACCTGAGCG | 17 | CGCTCAGGTGGAAATTACAA | 124 |
| SYNGAP1 E18 + 46 | GGCTCAGGCAGCGGCTCAGC | 18 | GCTGAGCCGCTGCCTGAGCC | 125 |
| SYNGAP1 I18 + 0 | AATGACATTGTAATTTCCAC | 19 | GTGGAAATTACAATGTCATT | 126 |
| SYNGAP1 I18 + 5 | AGATAAATGACATTGTAATT | 20 | AATTACAATGTCATTTATCT | 127 |
| SYNGAP1 I18 + 10 | GGAGAAGATAAATGACATTG | 21 | CAATGTCATTTATCTTCTCC | 128 |
| SYNGAP1 I18 + 20 | GATGGGACACGGAGAAGATA | 22 | TATCTTCTCCGTGTCCCATC | 129 |
| SYNGAP1 I18 + 30 | 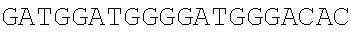 | 23 | GTGTCCCATCCCCATCCATC | 130 |
| SYNGAP1 I18 + 40 | AAGACAGTGGGATGGATGGG | 24 | CCCATCCATCCCACTGTCTT | 131 |
| SYNGAP1 I18 + 50 | GAGTGCACGAAAGACAGTGG | 25 | CCACTGTCTTTCGTGCACTC | 132 |
| SYNGAP1 I18 + 60 | CTGGTGTAGTGAGTGCACGA | 26 | TCGTGCACTCACTACACCAG | 133 |
| SYNGAP1 I18 + 70 | GGCTAGGTGGCTGGTGTAGT | 27 | ACTACACCAGCCACCTAGCC | 134 |
| SYNGAP1 E18 + 64 19 mer | TCGAGCAGCCTCTTCTTGG | 71 | CCAAGAAGAGGCTGCTCGA | 135 |
| SYNGAP1 E18 + 66 19 mer | CGTCGAGCAGCCTCTTCTT | 72 | AAGAAGAGGCTGCTCGACG | 136 |
| SYNGAP1 E18 + 69 19 mer | GAGCGTCGAGCAGCCTCTT | 73 | AAGAGGCTGCTCGACGCTC | 137 |
| SYNGAP1 E18 + 71 18 mer | TGAGCGTCGAGCAGCCTC | 74 | GAGGCTGCTCGACGCTCA | 138 |
| SYNGAP1 E18 + 74 18 mer | ACCTGAGCGTCGAGCAGC | 75 | GCTGCTCGACGCTCAGGT | 139 |
| SYNGAP1 E18 + 79 | AATTTCCACCTGAGCGTCGA | 76 | TCGACGCTCAGGTGGAAATT | 140 |
| SYNGAP1 E18 + 82 | TGTAATTTCCACCTGAGCGT | 77 | ACGCTCAGGTGGAAATTACA | 141 |
| SYNGAP1 E18 + 86 22 mer | TGACATTGTAATTTCCACCTGA | 78 | TCAGGTGGAAATTACAATGTCA | 142 |
| SYNGAP1 I18 + 2 22 mer | GATAAATGACATTGTAATTTCC | 79 | GGAAATTACAATGTCATTTATC | 143 |
| SYNGAP1 I18 + 8 22 mer | GGAGAAGATAAATGACATTGTA | 80 | TACAATGTCATTTATCTTCTCC | 144 |
| SYNGAP1 I18 + 13 | CACGGAGAAGATAAATGACA | 81 | TGTCATTTATCTTCTCCGTG | 145 |
| SYNGAP1 I18 + 15 | GACACGGAGAAGATAAATGA | 82 | TCATTTATCTTCTCCGTGTC | 146 |
| SYNGAP1 I18 + 16 | GGACACGGAGAAGATAAATG | 83 | CATTTATCTTCTCCGTGTCC | 147 |
| SYNGAP1 I18 + 17 | GGGACACGGAGAAGATAAAT | 84 | ATTTATCTTCTCCGTGTCCC | 148 |
| SYNGAP1 I18 + 18 | TGGGACACGGAGAAGATAAA | 85 | TTTATCTTCTCCGTGTCCCA | 149 |
| SYNGAP1 I18 + 18 22 mer | GATGGGACACGGAGAAGATAAA | 86 | TTTATCTTCTCCGTGTCCCATC | 150 |
| SYNGAP1 I18 + 19 | ATGGGACACGGAGAAGATAA | 87 | TTATCTTCTCCGTGTCCCAT | 151 |
| SYNGAP1 I18 + 19 21 mer | GATGGGACACGGAGAAGATAA | 88 | TTATCTTCTCCGTGTCCCATC | 152 |
| SYNGAP1 I18 + 20 21 mer | GGATGGGACACGGAGAAGATA | 89 | TATCTTCTCCGTGTCCCATCC | 153 |
| SYNGAP1 I18 + 20 22 mer | GGGATGGGACACGGAGAAGATA | 90 | TATCTTCTCCGTGTCCCATCCC | 154 |
| SYNGAP1 I18 + 21 | GGATGGGACACGGAGAAGAT | 91 | ATCTTCTCCGTGTCCCATCC | 155 |
| SYNGAP1 I18 + 22 19 mer | GGATGGGACACGGAGAAGA | 92 | TCTTCTCCGTGTCCCATCC | 156 |
| SYNGAP1 I18 + 23 18 mer | GGATGGGACACGGAGAAG | 93 | CTTCTCCGTGTCCCATCC | 157 |
| SYNGAP1 I18 + 24 18 mer | GGGATGGGACACGGAGAA | 94 | TTCTCCGTGTCCCATCCC | 158 |
| SYNGAP1 I18 + 20 19 mer | ATGGGACACGGAGAAGATA | 95 | TATCTTCTCCGTGTCCCAT | 159 |
| SYNGAP1 I18 + 21 19 mer | GATGGGACACGGAGAAGAT | 96 | ATCTTCTCCGTGTCCCATC | 160 |
| SYNGAP1 I18 + 20 18 mer | TGGGACACGGAGAAGATA | 97 | TATCTTCTCCGTGTCCCA | 161 |
| SYNGAP1 I18 + 21 18 mer | ATGGGACACGGAGAAGAT | 98 | ATCTTCTCCGTGTCCCAT | 162 |
| SYNGAP1 I18 + 22 18 mer | GATGGGACACGGAGAAGA | 99 | TCTTCTCCGTGTCCCATC | 163 |
| SYNGAP1 I18 + 20 17 mer | GGGACACGGAGAAGATA | 100 | TATCTTCTCCGTGTCCC | 164 |
| SYNGAP1 I18 + 21 17 mer | TGGGACACGGAGAAGAT | 101 | ATCTTCTCCGTGTCCCA | 165 |
| SYNGAP1 I18 + 22 17 mer | ATGGGACACGGAGAAGA | 102 | TCTTCTCCGTGTCCCAT | 166 |
| SYNGAP1 I18 + 23 17 mer | GATGGGACACGGAGAAG | 103 | CTTCTCCGTGTCCCATC | 167 |
| SYNGAP1 I18 + 20 16 mer | GGACACGGAGAAGATA | 104 | TATCTTCTCCGTGTCC | 168 |
| SYNGAP1 I18 + 21 16 mer | GGGACACGGAGAAGAT | 105 | ATCTTCTCCGTGTCCC | 169 |
| SYNGAP1 I18 + 22 16 mer | TGGGACACGGAGAAGA | 106 | TCTTCTCCGTGTCCCA | 170 |
| SYNGAP1 I18 + 23 16 mer | ATGGGACACGGAGAAG | 107 | CTTCTCCGTGTCCCAT | 171 |
| SYNGAP1 I18 + 24 16 mer | GATGGGACACGGAGAA | 108 | TTCTCCGTGTCCCATC | 172 |
| SYNGAP1 I18 + 20 15 mer | GACACGGAGAAGATA | 109 | TATCTTCTCCGTGTC | 173 |
| SYNGAP1 I18 + 21 15 mer | GGACACGGAGAAGAT | 110 | ATCTTCTCCGTGTCC | 174 |
| SYNGAP1 I18 + 22 15 mer | GGGACACGGAGAAGA | 111 | TCTTCTCCGTGTCCC | 175 |
| SYNGAP1 I18 + 23 15 mer | TGGGACACGGAGAAG | 112 | CTTCTCCGTGTCCCA | 176 |
| SYNGAP1 I18 + 24 15 mer | ATGGGACACGGAGAA | 113 | TTCTCCGTGTCCCAT | 177 |
| SYNGAP1 I18 + 25 15 mer | GATGGGACACGGAGA | 114 | TCTCCGTGTCCCATC | 178 |
| SYNGAP1 E19 SA(−11) | GATGAGGAGCTGCAGTGGGA | 28 | TCCCACTGCAGCTCCTCATC | 179 |
| SYNGAP1 E19 SA(−3) | AATTACCTGATGAGGAGCTG | 29 | CAGCTCCTCATCAGGTAATT | 180 |
| SYNGAP1 E19 + 6 | ACCAGGAGAATTACCTGATG | 30 | CATCAGGTAATTCTCCTGGT | 181 |
| SYNGAP1 E19 + 31 | CCTCCGCCCGTGGCCAAA | 31 | TTTGGCCACGGGCGGAGG | 182 |
| SYNGAP1 E19 + 38 | CCTGTGTCCTCCGCCCGT | 32 | ACGGGCGGAGGACACAGG | 183 |
| SYNGAP1 E19 + 56 | TGGTCCGGAGTCACCTCCC | 33 | GGGAGGTGACTCCGGACCA | 184 |
| SYNGAP1 E19 + 62 | CTGCAGTGGTCCGGAGTCA | 34 | TGACTCCGGACCACTGCAG | 185 |
| SYNGAP1 E20 SA(−87) | CCTAGCAGTGGCCTCCCCTA | 35 | TAGGGGAGGCCACTGCTAGG | 186 |
| SYNGAP1 E20 SA(−77) | ATGCCAGTCCCCTAGCAGTG | 36 | CACTGCTAGGGGACTGGCAT | 187 |
| SYNGAP1 E20 SA(−11) | CTGCCTCTCCTGCAAGACAC | 37 | GTGTCTTGCAGGAGAGGCAG | 188 |
| SYNGAP1 E20 + 4 | 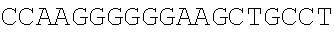 | 38 | AGGCAGCTTCCCCCCTTGG | 189 |
| SYNGAP1 E20 + 16 | GGTTTGTTGGACCCAAGGG | 39 | CCCTTGGGTCCAACAAACC | 190 |

* BOLD ASOs are alpha1-increasing top hits
* Italicized ASOs are alpha1-decreasing top hits
* Highlighted bases indicate G-quadruplex sequences

When one or more agents that can modulate SYNGAP1 splicing (e.g., one or more SSOs) described herein are used to treat a mammal (e.g., a human) having, or at risk of developing a SYNGAP1-associated NDD, any type of mammal having, or at risk for developing, a SYNGAP1-associated NDD (e.g., SRID) can be treated as described herein. In some cases, a mammal to be treated as described herein can be an infant (e.g., a newborn). In some cases, a mammal to be treated as described herein can be in utero. Examples of mammals that can have, or can be at risk for developing, a SYNGAP1-associated NDD (e.g., SRID) and can be treated as described herein include, without limitation, humans, non-human primates such as monkeys, dogs, cats, horses, cows, pigs, sheep, rabbits, mice, and rats.

In some cases, methods described herein can include identifying a mammal (e.g., a human) as having or being at risk of developing pancreatitis (e.g., chronic pancreatitis). Any appropriate method can be used to identify a mammal as having, or at risk for developing, a SYNGAP1-associated NDD (e.g., SRID). For example, genetic testing, EEG, and/or epilepsy can be used to identify a human or other mammal as having, or at risk for developing, a SYNGAP1-associated NDD (e.g., SRID).

When one or more agents that can modulate SYNGAP1 splicing (e.g., one or more SSOs) described herein are used to treat a mammal (e.g., a human) having, or at risk of developing a SYNGAP1-associated NDD, the SYNGAP1-associated NDD can be any appropriate SYNGAP1-associated NDD. Examples of SYNGAP1-associated NDD that can be treated as described herein (e.g., by administering one or more agents that can modulate SYNGAP1 splicing such as one or more SSOs described herein) include, without limitation, SRID, SYNGAP1-related autism spectrum disorder (ASD), SYNGAP1-related epilepsy, sleep disorders, intellectual disability, and schizophrenia.

When one or more agents that can modulate SYNGAP1 splicing (e.g., one or more SSOs) are used to treat a mammal (e.g., a human) having, or at risk of developing a SYNGAP1-associated NDD, the mammal (e.g., a human) having, or at risk for developing, a SYNGAP1-associated NDD (e.g., SRID) can be administered or instructed to self-administer any one or more agents that can modulate SYNGAP1 splicing (e.g., one or more SSOs) described herein.

In some cases, one or more agents that can modulate SYNGAP1 splicing (e.g., one or more SSOs) can be used to reduce or eliminate the level of a SynGAP1-β polypeptide and/or a SynGAP1-γ polypeptide within neurons within a mammal (e.g., a human). For example, one or more agents that can modulate SYNGAP1 splicing (e.g., one or more SSOs) can be administered to a mammal (e.g., a human) in need thereof (e.g., a human having, or at risk for developing, a SYNGAP1-associated NDD such as SRID) to reduce or eliminate the level of a SynGAP1-β polypeptide and/or a SynGAP1-γ polypeptide within neurons within the mammal. The term “reduced level” as used herein with respect to a level of a SynGAP1-β polypeptide and/or a SynGAP1-γ polypeptide in a mammal having, or at risk for developing, a SYNGAP1-associated NDD (e.g., SRID) refers to any level that is lower than the level of that SynGAP1 polypeptide observed in that mammal prior to being treated as described herein. In some cases, a reduced level of a SynGAP1-β polypeptide and/or a SynGAP1-γ polypeptide can be a level that is at least 5 percent (e.g., at least 10, at least 15, at least 20, at least 25, at least 35, at least 50, at least 75, at least 100, or at least 150 percent) less than the level of that SynGAP1 polypeptide prior to being treated as described herein. It will be appreciated that levels from comparable samples are used when determining whether or not a particular level is an increased level.

In some cases, one or more agents that can modulate SYNGAP1 splicing (e.g., one or more SSOs) can be used to increase the level of SynGAP1-α1 polypeptides and/or SynGAP1-α2 polypeptides within neurons within a mammal (e.g., a human). For example, one or more agents that can modulate SYNGAP1 splicing (e.g., one or more SSOs) can be administered to a mammal (e.g., a human) in need thereof (e.g., a human having, or at risk for developing, a SYNGAP1-associated NDD such as SRID) to increase the level of a SynGAP1-α1 polypeptide and/or a SynGAP1-α2 polypeptide within neurons within the mammal. The term “increased level” as used herein with respect to a level of a SynGAP1-α1 polypeptide and/or a SynGAP1-α2 polypeptide in a mammal having, or at risk for developing, a SYNGAP1-associated NDD (e.g., SRID) refers to any level that is greater than the level of that SynGAP1 polypeptide observed in that mammal prior to being treated as described herein. In some cases, an increased level of a Syngap1-α1 polypeptide and/or a SynGAP1-α2 polypeptide can be a level that is at least 5 percent (e.g., at least 10, at least 15, at least 20, at least 25, at least 35, at least 50, at least 75, at least 100, or at least 150 percent) higher than the level of that SynGAP1 polypeptide prior to being treated as described herein. In some cases, when samples have an undetectable level of a Syngap1-α1 polypeptide prior to treatment as described herein, an increased level can be any detectable level of a Syngap1-α1 polypeptide and/or a SynGAP1-α2 polypeptide. It will be appreciated that levels from comparable samples are used when determining whether or not a particular level is an increased level.

In some cases, one or more agents that can modulate SYNGAP1 splicing (e.g., one or more SSOs) described herein can be used to reduce the severity of one or more symptoms of a SYNGAP1-associated NDD (e.g., SRID). For example, one or more agents that can modulate SYNGAP1 splicing can be administered to a mammal (e.g., a human) in need thereof (e.g., a human having or at risk of developing a SYNGAP1-associated NDD (e.g., SRID)) to reduce the severity of one or more symptoms of the SYNGAP1-associated NDD (e.g., SRID). Examples of symptoms of a SYNGAP1-associated NDD (e.g., SRID) include, without limitation, gross motor delays (e.g., in infancy), developmental delays, seizures, language impairment, sleep disorders, and intellectual disability. In some cases, the methods and materials described herein can be effective to reduce the severity of one or more symptoms of a SYNGAP1-associated NDD (e.g., SRID) in a mammal having SYNGAP1-associated NDD (e.g., SRID) by, for example, 10, 20, 30, 40, 50, 60, 70, 80, 90, 95, or more percent.

In some cases, one or more agents that can modulate SYNGAP1 splicing (e.g., one or more SSOs) described herein can be used to reduce or slow the progression of a SYNGAP1-associated NDD (e.g., SRID). For example, one or more agents that can modulate SYNGAP1 splicing can be administered to a mammal (e.g., a human) in need thereof (e.g., a human having a SYNGAP1-associated NDD such as SRID) to reduce or slow the progression of a SYNGAP1-associated NDD (e.g., SRID) in the mammal. In some cases, the methods and materials described herein can be effective to reduce or slow the progression of a SYNGAP1-associated NDD (e.g., SRID) in a mammal having a SYNGAP1-associated NDD (e.g., SRID) by, for example, 10, 20, 30, 40, 50, 60, 70, 80, 90, 95, or more percent. In some cases, the methods and materials described herein can be effective to reduce or slow the progression of a SYNGAP1-associated NDD (e.g., SRID) in a mammal having a SYNGAP1-associated NDD (e.g., SRID) by, for example, at least 6 months (e.g., about 6 months, about 8 months, about 10 months, about 1 year, about 1.5 years, about 2 years, about 2.5 years, about 3 years, about 4 years, about 5 years, or more).

In some cases, one or more agents that can modulate SYNGAP1 splicing (e.g., one or more SSOs) can be used to delay or prevent the development of a SYNGAP1-associated NDD (e.g., SRID). For example, one or more agents that can modulate SYNGAP1 splicing can be administered to a mammal (e.g., a human) in need thereof (e.g., a human at risk of developing a SYNGAP1-associated NDD such as SRID) to delay or prevent the development of a SYNGAP1-associated NDD (e.g., SRID) in the mammal. In some cases, the methods and materials described herein can be effective to delay the development of a SYNGAP1-associated NDD (e.g., SRID) in a mammal at risk of developing a SYNGAP1-associated NDD (e.g., SRID) by, for example, 10, 20, 30, 40, 50, 60, 70, 80, 90, 95, or more percent. In some cases, the methods and materials described herein can be effective to delay the development of a SYNGAP1-associated NDD (e.g., SRID) in a mammal at risk of developing a SYNGAP1-associated NDD (e.g., SRID) by, for example, at least 6 months (e.g., about 6 months, about 8 months, about 10 months, about 1 year, about 1.5 years, about 2 years, about 2.5 years, about 3 years, about 4 years, about 5 years, or more).

A composition containing one or more (e.g., one, two, three, four, or more) agents that can modulate SYNGAP1 splicing (e.g., one or more SSOs) described herein can be administered to a mammal (e.g., a human) having, or at risk for developing, a SYNGAP1-associated NDD (e.g., SRID) in any appropriate amount (e.g., any appropriate dose). An effective amount of a composition containing one or more agents that can modulate SYNGAP1 splicing can be any amount that can treat a mammal having, or at risk for developing, a SYNGAP1-associated NDD (e.g., SRID) as described herein without producing significant toxicity to the mammal. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and/or severity of the pancreatitis (e.g., chronic pancreatitis) in the mammal being treated may require an increase or decrease in the actual effective amount administered.

A composition containing one or more (e.g., one, two, three, four, or more) agents that can modulate SYNGAP1 splicing (e.g., one or more SSOs) described herein can be administered to a mammal (e.g., a human) having, or at risk for developing, a SYNGAP1-associated NDD (e.g., SRID) in any appropriate frequency. The frequency of administration can be any frequency that can treat a mammal having, or at risk for developing, a SYNGAP1-associated NDD (e.g., SRID) without producing significant toxicity to the mammal. For example, the frequency of administration can be from about twice a day to about one every other day, once a day to about once a week, from about once a week to about once a month, or from about twice a month to about once a month. The frequency of administration can remain constant or can be variable during the duration of treatment. As with the effective amount, various factors can influence the actual frequency of administration used for a particular application. For example, the effective amount, duration of treatment, use of multiple treatment agents, and/or route of administration may require an increase or decrease in administration frequency.

A composition containing one or more (e.g., one, two, three, four, or more) agents that can modulate SYNGAP1 splicing (e.g., one or more SSOs) described herein can be administered to a mammal (e.g., a human) having, or at risk for developing, a SYNGAP1-associated NDD (e.g., SRID) for any appropriate duration. An effective duration for administering or using a composition containing one or more agents that can modulate SYNGAP1 splicing can be any duration that can treat a mammal having, or at risk for developing, a SYNGAP1-associated NDD (e.g., SRID) without producing significant toxicity to the mammal. For example, the effective duration can vary from several weeks to several months, from several months to several years, or from several years to a lifetime. Multiple factors can influence the actual effective duration used for a particular treatment. For example, an effective duration can vary with the frequency of administration, effective amount, use of multiple treatment agents, and/or route of administration.

In some cases, methods for treating a mammal (e.g., a human) having, or at risk for developing, a SYNGAP1-associated NDD (e.g., SRID) as described herein (e.g., by administering one or more agents that can modulate SYNGAP1 splicing such as one or more SSOs described herein) can include administering to the mammal one or more (e.g., one, two, three, four, or more) agents that can modulate SYNGAP1 splicing (e.g., one or more SSOs) as the sole active ingredient to treat the mammal. For example, a composition containing one or more agents that can modulate SYNGAP1 splicing described herein can include the agent(s) that can modulate SYNGAP1 splicing as the sole active ingredient in the composition that is effective to treat a mammal having, or at risk for developing, a SYNGAP1-associated NDD (e.g., SRID).

In some cases, methods for treating a mammal (e.g., a human) having, or at risk for developing, a SYNGAP1-associated NDD (e.g., SRID) as described herein (e.g., by administering one or more agents that can modulate SYNGAP1 splicing such as one or more SSOs described herein) also can include administering to the mammal one or more (e.g., one, two, three, four, five or more) additional agents used to treat SYNGAP1-associated NDD (e.g., SRID) to the mammal and/or performing therapies used to treat SYNGAP1-associated NDD (e.g., SRID) on the mammal. For example, a combination therapy used to treat SYNGAP1-associated NDD (e.g., SRID) can include administering to the mammal (e.g., a human) one or more agents that can modulate SYNGAP1 splicing described herein and one or more (e.g., one, two, three, four, five or more) agents used to treat SYNGAP1-associated NDD (e.g., SRID). Examples of agents that can be administered to a mammal to treat SYNGAP1-associated NDD (e.g., SRID) include, without limitation, anti-seizure agents, antipsychotic agents (e.g., Risperdal), ADHD treatments (e.g., Guanfacine), sleep disorder treatments, and any combinations thereof. In cases where one or more agents that can modulate SYNGAP1 splicing described herein are used in combination with additional agents used to treat SYNGAP1-associated NDD (e.g., SRID), the one or more additional agents can be administered at the same time (e.g., in a single composition containing both one or more agents that can modulate SYNGAP1 splicing described herein and the one or more additional agents) or independently. For example, one or more agents that can modulate SYNGAP1 splicing described herein can be administered first, and the one or more additional agents administered second, or vice versa.

In some cases, a combination therapy used to treat SYNGAP1-associated NDD (e.g., SRID) can include administering to the mammal (e.g., a human) one or more (e.g., one, two, three, four, or more) agents that can modulate SYNGAP1 splicing (e.g., one or more SSOs) and performing one or more (e.g., one, two, three, four, five or more) additional therapies used to treat SYNGAP1-associated NDD (e.g., SRID) on the mammal. Examples of therapies used to treat SYNGAP1-associated NDD (e.g., SRID) include, without limitation, occupational therapy, physical therapy, speech and language therapy, applied behavioral analysis therapy, and/or developmental therapy. In cases where one or more agents that can modulate SYNGAP1 splicing described herein are used in combination with one or more additional therapies used to treat SYNGAP1-associated NDD (e.g., SRID), the one or more additional therapies can be performed at the same time or independently of the administration of one or more agents that can modulate SYNGAP1 splicing described herein. For example, one or more viral vectors provided herein can be administered before, during, or after the one or more additional therapies are performed.

In certain instances, a course of treatment and the severity of one or more symptoms related to the condition being treated (e.g., a SYNGAP1-associated NDD such as SRID) can be monitored. Any appropriate method can be used to determine whether or not the severity of a symptom is reduced. For example, the severity of a symptom of a SYNGAP1-associated NDD (e.g., SRID) can be assessed using EEG, epilepsy measurements, hyperactivity, working memory behavior, biomarker assays, sleep measurements, and/or behavioral assessments at different time points.

The invention will be further described in the following examples, which do not limit the scope of the invention described in the claims.

### EXAMPLES

### Example 1: Modulation of SYNGAP1 Non-Canonical Splicing and Development of Anti-Sense Oligonucleotides (ASOs) for SYNGAP1 Haploinsufficiency Treatment and Other SynGAP-Related Developmental Disorders

This Example describes the design and generation of chromatic splice reporter nucleic acid constructs (e.g., chromatic splice reporter plasmids) that can be used to distinguish which isoform of a SynGAP1 polypeptide is being expressed by a cell, and can be used to screen for agents that can modulate SYNGAP1 splicing (e.g., splice-switching ASOs (SSOs)). For example, splice reporter nucleic acid constructs described herein can be used screen for SSOs that can suppress expression of a SynGAP1-α2 polypeptide, a SynGAP1-α3 polypeptide, a SynGAP1-β polypeptide, and/or a SynGAP1-γ polypeptide. For example, splice reporter nucleic acid constructs described herein can be used screen for SSOs that can increase expression of a SynGAP1-α1 polypeptide and/or a SynGAP1-α2 polypeptide.

SYNGAP1-α1 Arises from a Non-Canonical Splicing

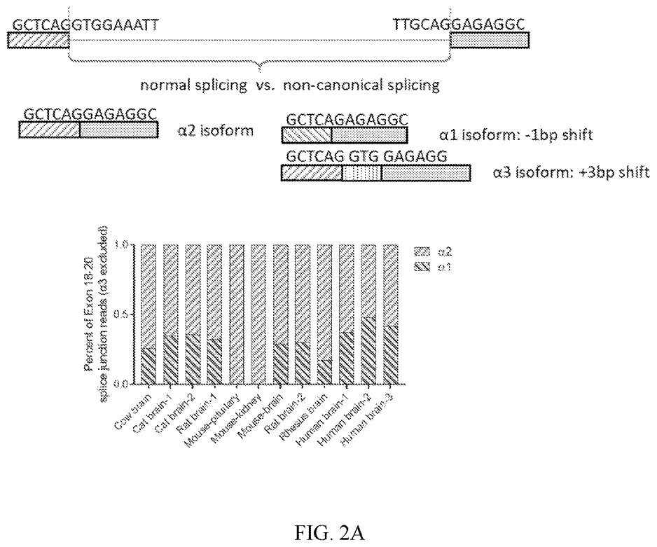

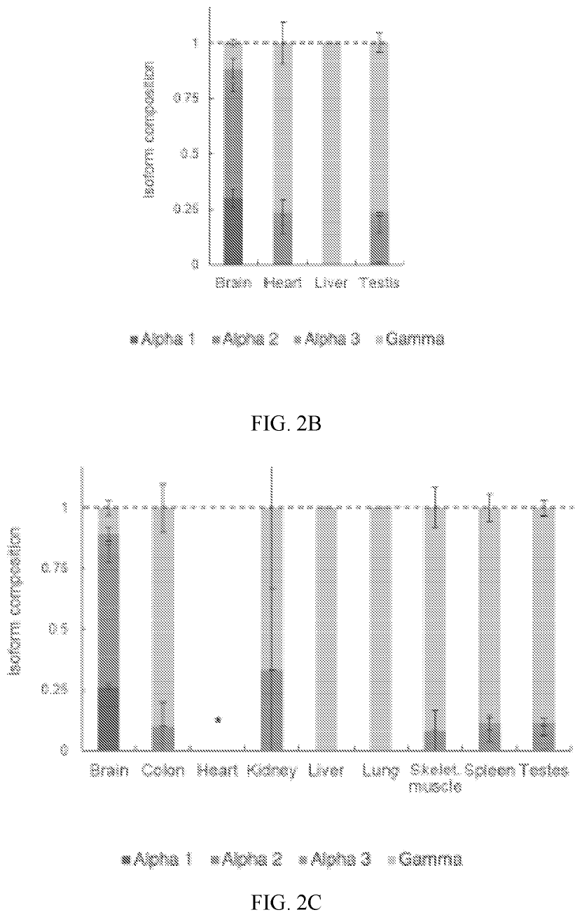

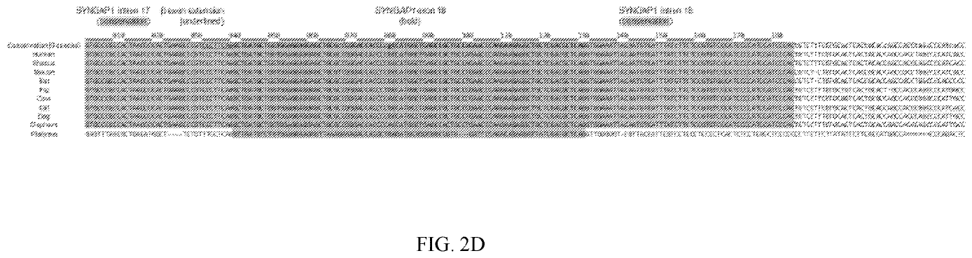

It was discovered that SynGAP-α1 arises from a non-canonical splicing event (GG-AG, FIG. 2A, Table 2, and Table 3) that is unique to brain cells (FIGS. 2B and 2C) and conserved across many mammalian species (FIG. 2D).

Splice junctions and corresponding c-terminal isoforms in human SYNGAP1 are shown in Table 2. The non-canonical nature of SynGAP-α1 and SynGAP-α3, and the ambiguity of the correct alignment for sequenced mRNA junction reads lead to difficulties in quantifying the splicing. Through mutational analysis, it was established that the non-canonical splicing occurs through deletion or addition of bases from the splice donor site (hg38 chr6:33447934). Most current transcriptomes contain annotation for the SynGAP-α1 non-canonical junction, but SynGAP-α3 has not been annotated yet. Providing these junction annotations at alignment time allows accurate quantification of RNA-seq reads. The SynGAP-α1 non-canonical splicing gives rise to a unique c-terminal sequence which includes a PDZ ligand that contributes to PSD-95 binding.

TABLE 2
Human SYNGAP1 splice junctions between exon 18/19/20
and corresponding c-terminal isoforms

| Isoform | Junction (hg38) | Can be misaligned to: | Splice event |
|---|---|---|---|
| α1 | chr6: 33447933-33451759 | chr6: 33447934-33451760 | non-canonical GG-AG |
| α2 | chr6: 33447934-33451759 | | canonical GT-AG |
| α3 | chr6: 33447937-33451759 | chr6: 33447936-33451758 | non-canonical GA-AG |
| γ | chr6: 33447934-33448788 | | canonical GT-AG |

Upstream of this junction, an exon extension at the 5′ of exon 18 is alternatively spliced to give rise of SynGAP-β and non-β isoforms (Table 3). Exons 17 and 18 encode a coiled coil domain critical for trimerization and interaction with PSD-95, and phase separation. The SynGAP-β isoform terminates within exon 18, leading to a truncated coiled-coil domain and substantially lower synaptic localization (see, e.g., Araki et al., eLife, 9: e56273 (2020)). This stop codon is within 43 nucleotides of the 3′ exon junction, which could trigger NMD especially when downstream splicing includes exon 19 (γ), due to a sufficient distance from the 3′ most junction of the transcript (43+81 bp>50 bp).

TABLE 3
Human SYNGAP1 splice junctions between exon
17/18 and corresponding c-terminal isoforms

| Isoform | Junction (hg38) | Outcome | Splice event |
|---|---|---|---|
| β | chr6: 33446787-33447829 | Truncated coiled-coil domain | Exon extension |
| Non-β | chr6: 33446787-33447842 | Intact coiled-coil domain | α1/α2/α3/γ |

SynGAP-α1 non-canonical splicing (NCS) is highly dependent on the first 54 bases of the intervening intron, which is highly conserved across mammalian species, spanning elephants and dogs to humans (FIG. 2D). Transcriptome-wide searches verified several splice junctions where a non-canonical GG-AG splice junction gave 1 bp shorter transcripts compared to the canonical GT-AG junction, but all to a much lesser extent compared to SYNGAP1 (Table 4). The SynGAP-α1 non-canonical splicing gives rise to a unique c-terminal sequence which includes a PDZ ligand that contributes to PSD-95 binding.

A novel SynGAP-α3 isoform was also identified, which retains 3 additional bases from the splice donor site (‘GTG’) compared to SynGAP-α2 and like SynGAP-α1 arises from a noncanonical donor-acceptor pair (GA-AG, FIG. 2B). This isoform only occurs at the exon 18-20 junction and would retain the reading frame of SynGAP-α2 in exon 20.

The noncanonical nature of SynGAP-α1 splicing, as well as its unique frame-shifting impact on protein function, prompted a search for similar phenomena across the genome (Table 4). A mouse and human transcriptome-wide search for Gg(t)-ag or gt-(a)gG splice sites, and a more general search for all 1 bp-shifted splice sites (in mouse brain samples) both led to only splice sites with agG splice acceptors. This suggested that the ‘G’ in these ag′G′ splice acceptor sites might be the nucleotide that is removed during the NCS.

SYNGAP1 and LDB1 were the only two genes to display >10% levels of 1 bp-shifted NCS, suggesting that the SYNGAP1 α1 NCS mechanism is highly specific to a small number of genes. The two genes showed NCS that is conserved in mice and humans, and have strong predicted functional outcomes with the ensuing frameshift. The LDB1 gene NCS junction had two canonical splice donor sites 6 bp apart that both displayed SYNGAP1 α1-like splicing that resulted in a missing ‘G’. Both of these LDB1 NCS products will lead to protein without the critical c-terminal LIM2-binding domain, which should impair the protein's transcriptional activity. The other genes displayed lower levels of α1-like NCS and low levels of expression in the brain.

TABLE 4
Transcriptome-wide search for SYNGAP1 α1-like 1 bp-shifted NCS.

| | | | | SEQ | | SEQ | | | | |
|---|---|---|---|---|---|---|---|---|---|---|
| | | Intron | Donor | ID | | ID | Anno- | Functional | Validation | Validation |
| Gene | Location | retention | (Canonical) | NO | Acceptor | NO | tation | outcome | Human hg38 | Mouse mm10 |
| SYNGAP1 | Last; | yes | GCTCAGgtggaa | 5 | ttgcagGA | 7 | yes | PDZ ligand | Verified in | SRR11596211 |
| | last-2 | | | | GAGG | | (RefSeq) | (alpha1) | SRR7368895 | |
| | exon | | | | | | | | | |
| LDB1 | Last; | yes | GTACCTgtaagc | 40 | tctcagGA | 41 | no | No LIM2- | SRR7368895 | SRR8425025 |
| | last-1 | | AGCCAGgtacct | 42 | TGTG | | | binding | | |
| | exon | | | | tctcagGA | 41 | ″ | domains | | |
| | | | | | TGTG | | | (Ldb1b) | | |
| NAA10 | Last-1; | yes | GAAGAGgtggaa | 43 | ccccagGA | 44 | yes | | SRR7368895 | |
| | last-3 | | | | TCAG | | FL-CDS | | | |
| | exon | | | | | | (MGC) | | | |
| CERCAM | Mid-gene | no | GCCCAGgtggtg | 45 | cctcagGT | 46 | no | | SRR7368895 | |
| | | | | | TCTA | | | | | |
| HERC2P2 | Mid-gene | no | GATCAGgtactc | 47 | tctcagGA | 48 | no | | SRR7368895 | |
| | | | | | TGGG | | | | | |
| HERC2P9 | | | GATCAGgtactc | 47 | tctcagGA | 48 | no | | SRR7368895 | |
| | | | | | TGGG | | | | | |
| NACC2 | | | | | | | | | Exp low in | |
| | | | | | | | | | brain | |
| AQP4 | | | | | | | | | Jxn rare in | |
| | | | | | | | | | brain | |
| Hyou1 | | | | | | | | | | can't |
| | | | | | | | | | | see in |
| | | | | | | | | | | SRR9591096 |
| Dpp10 | | | | | | | | | | can't |
| | | | | | | | | | | see in |
| | | | | | | | | | | SRR7425025 |
| Zdhhc8 | | | | | | | | | | can't see |
| | | | | | | | | | | in |
| | | | | | | | | | | SRR11596211 |
| Bcap31 | Last-1; | | AGAAAAgtgagg | 49 | ttcaagAA | 50 | | Insert, | can't | SRR11596211 |
| | last-2 | | | | CTAG | | | not | see in | (A/T insert) |
| | exon | | | | | | | deletion | SRR7368895 | |

| | Proteomic | CDNA | Relative | | Non-canonical | Canonical Intron | Adjacent |
|---|---|---|---|---|---|---|---|
| Gene | evidence | evidence | Level | Strand | Intron Coordinates | Coordinates | splice site |
| SYNGAP1 | yes | yes | high 32% | + | chr6:33447933- | chr6:33447934- | ‘GTG’ insert |
| | | | | | 33451759 | 33451759 | |
| LDB1 | no | no | med 15% | − | chr10:102108323- | chr10:102108324- | ‘GTACCT’ |
| | ″ | | | | 102109028 | 102109028 | deletion also |
| | | | | | chr10:102108323- | chr10:102108324- | showing NCS |
| | | | | | 102109034 | 102109034 | |
| NAA10 | | yes | low 4.6% | | chrX:153930847- | | |
| | | | | | 153932315 | | |
| CERCAM | | | low 3% | + | chr9:128422978- | | |
| | | | | | 128423145 | | |
| HERC2P2 | | | low 5.2% | + | chr15:22554572- | | |
| | | | | | 22556011 | | |
| HERC2P9 | | | low 5.7% | + | chr15:28649067- | | |
| | | | | | 28650506 | | |
| NACC2 | | | | | chr9:136024212- | | |
| | | | | | 136024249 | | |
| AQP4 | | | | − | chr18:26862474- | | |
| | | | | | 26865658 | | |
| Hyou1 | | | | | | mm10 chr9:44387092- | |
| | | | | | | 44387249 | |
| Dpp10 | | | | | | mm10 chr1:123402871- | |
| | | | | | | 123411710 | |
| Zdhhc8 | | | | | | mm10 chr16:18227874- | |
| | | | | | | 18228057 | |
| Bcap31 | | | med | | ambiguous | mm10 chrX:73687938- | |
| | | | 13.5% | | | 73688659 | |

*Shaded rows are junctions that are first found in rodents, and not observed in human samples.
**Bcap31 is unique from others in that a single bp ‘A’ insert is detected at a junction with multiple A's on both sides. This leads to an alignment with an ‘A’ insert at semi-random locations.
***hg38, mm10 unless noted otherwise
****SRR links lead to publicly available mouse/human brain RNAseq alignments
*****Underlined letters are in consensus with the SYNGAP1 junction. Red bases have been experimentally determined to be essential for NCS.

### Chromatic Reporter Plasmids

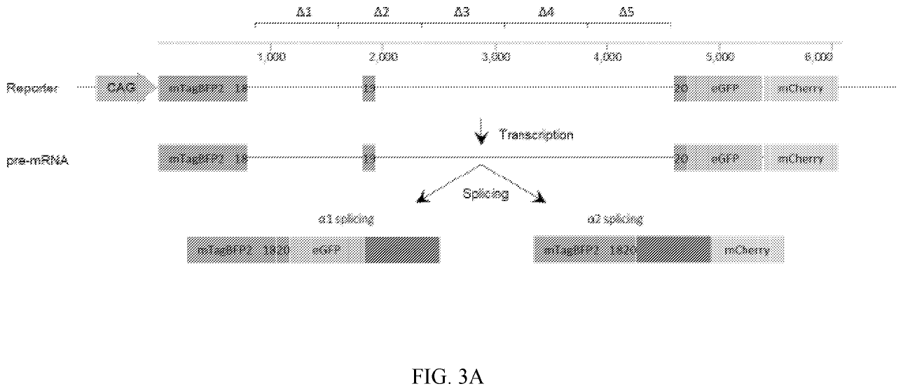

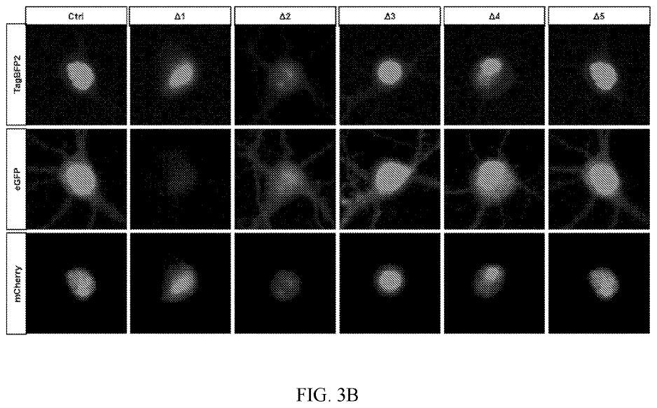

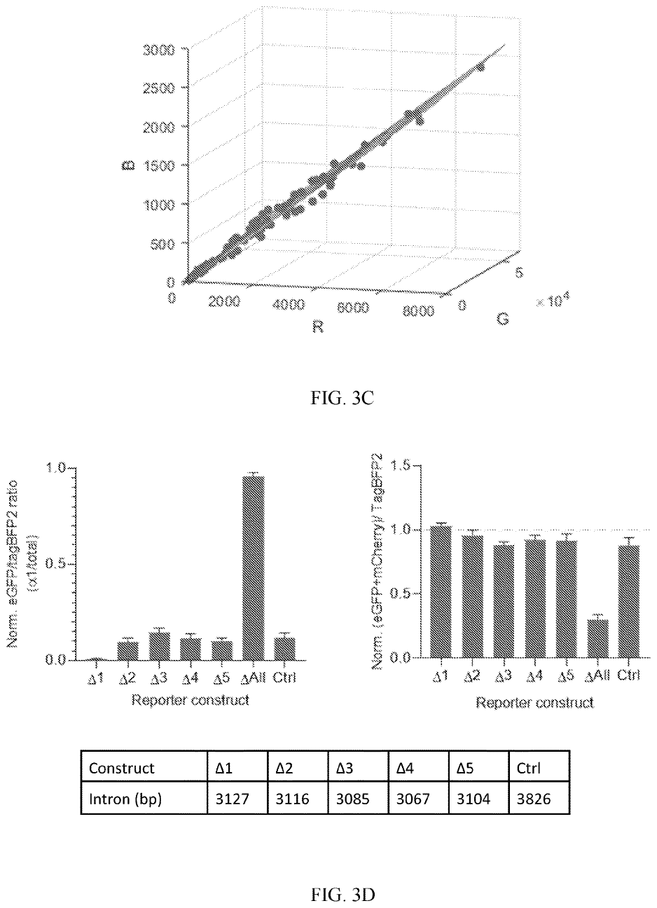

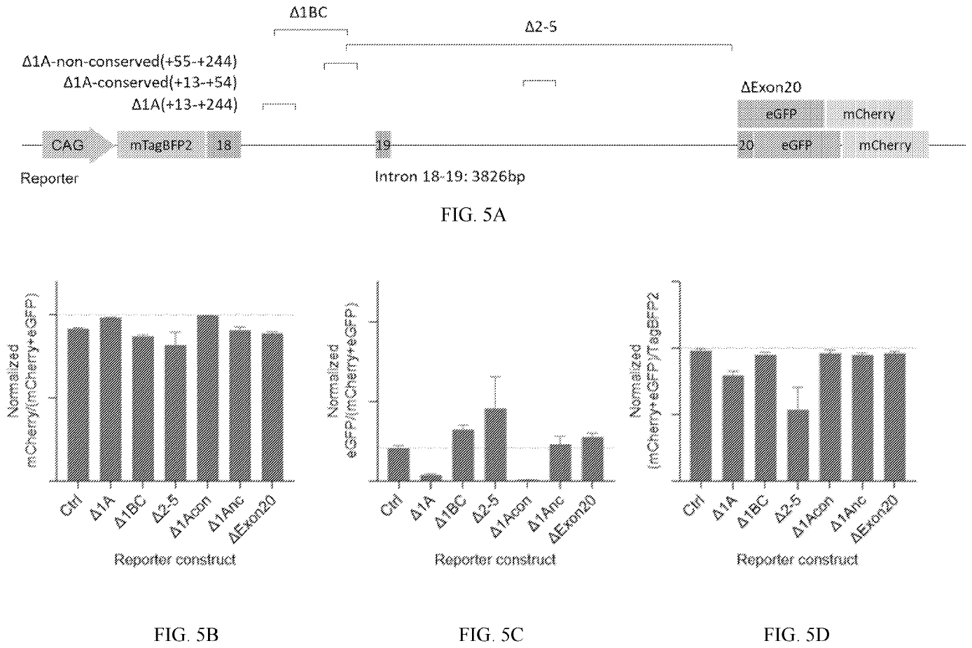

A SYNGAP1 minigene-based trichromatic and tetrachromatic splice reporters were developed and it was found that they reflect the normal SYNGAP1 splicing occurring in each cell (FIGS. 3-9). These reporters allow the identification of bases critical to each splice event, as well as the rapid screening of ASOs (anti-sense oligonucleotides) that can suppress SynGAP-α2, SynGAP-α3, SynGAP-β, and SynGAP-γ isoforms to, in turn, increase the SynGAP-α1 isoform expression.

To identify exonic/intronic sequences critical for the α1/α2/α3/γ splice decision (as well as β/non-β splice decision discussed later), a splice reporter construct was designed that included a CAG promoter, TagBFP2 constitutive fluorophore protein (FP), SYNGAP1 c-terminal minigene, eGFP, and mCherry, with eGFP and mCherry in different reading frames (FIG. 3A). The reporter takes advantage of the fact that the −1 reading frame of eGFP gene contains no stop codons to report the 1 bp difference in α1/α2 reading frame. When α1 splicing occurs, a stop codon arises at the end of the eGFP gene, and a TagBFP2-eGFP conjugated protein is expressed. Conversely, when α2 splicing occurs, a 1 bp frame-shift triggers the loss of the stop codon at the end of the eGFP gene and TagBFP2-mCherry conjugated protein is expressed instead.

In this trichromatic splice reporter including the exons and introns spanning the α1/α2/γ junction, systematic deletions were used to find that 800 bp at the beginning of intron 18 (Δ1) is essential for the alternative splicing of α1 (FIG. 3B). The blue, green and red fluorescence (FL) values of each cell, are designed to fall on a single plane when plotted in 3D space (FIG. 3C). A simple linear regression provides an estimate of coefficients κ1, κ2 that satisfy κ₁·green+κ₂·red≈blue FL. Using these coefficients, the normalized ratio of eGFP/TagBFP2 can be plotted as an estimate of the portion of α1 splicing in this minigene reporter. The results show the Δ1 intronic region is critical for α1 non-canonical splicing. A drop in the normalized (eGFP+mCherry)/TagBFP2 values would suggest TagBFP2-only expression likely arising from other frame-shifting splice sites including γ and cryptic splice sites. The normalized (eGFP+mCherry)/TagBFP2 values are relatively unchanged with the large intronic deletions, including Δ2 which includes the γ exon. This suggests that the occurrence of γ exon inclusion in this splice reporter is low, as in the endogenous SYNGAP1 gene in neurons (˜9%, FIGS. 2B and 2C).

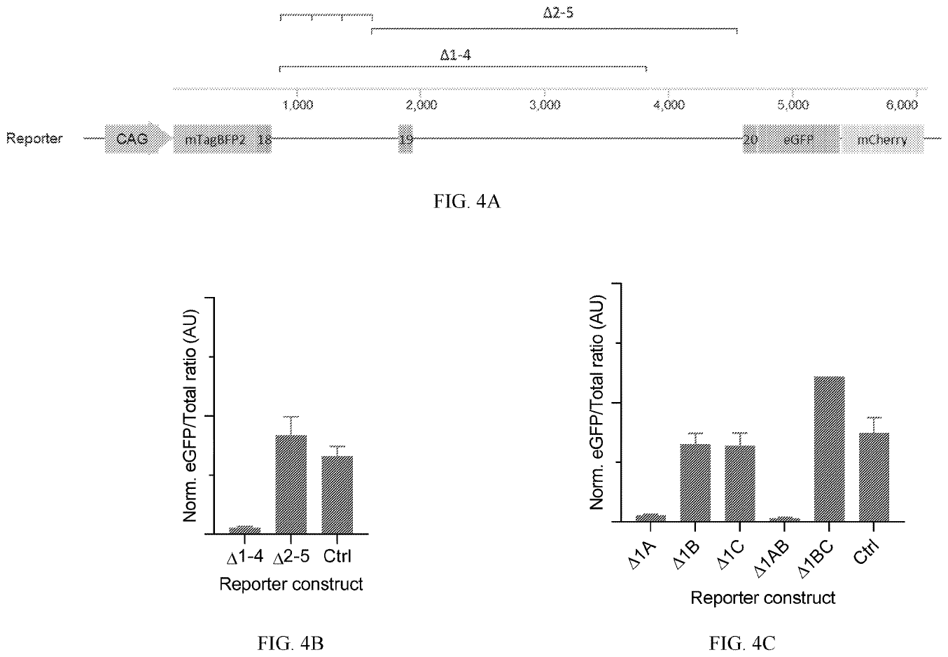

Large deletions were made that verified D2-5 region is dispensable for α1 splicing, whereas Δ1 is essential (FIG. 4B). Smaller deletions within the Δ1 region demonstrated that the 232 bp region at the beginning of the intron is crucial (Δ1A; +13-+244), while the remaining Δ1B and Δ1C regions are dispensable.

Further deletions were made to pinpoint a conserved intronic region (+13-+54) as the core determinant of α1 splicing (FIG. 4). Small deletions of this Δ1A-conserved region revealed that the conserved intronic region (FIG. 2D) is necessary for α1 non-canonical splicing, whereas Δ1A-non-conserved was dispensable. Δ1BC, Δ2-5 and ΔExon20 deletions increased α1:(α1+α2) ratio, suggesting a suppressive effect of these regions on α1 non-canonical splicing. It is currently unclear why Δ1BC had an effect on splicing with separate Δ1B and Δ1C deletions. Meanwhile the Δ1A and Δ2-5 constructs displayed a drop in (mCherry+eGFP)/TagBFP2, suggesting an increase in cryptic splicing or γ (exon 19) inclusion that leads to TagBFP2 expression alone. All together, these results point to the Δ1BC and ΔExon20 (37 bp) regions as priority targets for ASO screens to discover SSOs that increase α1 splicing.

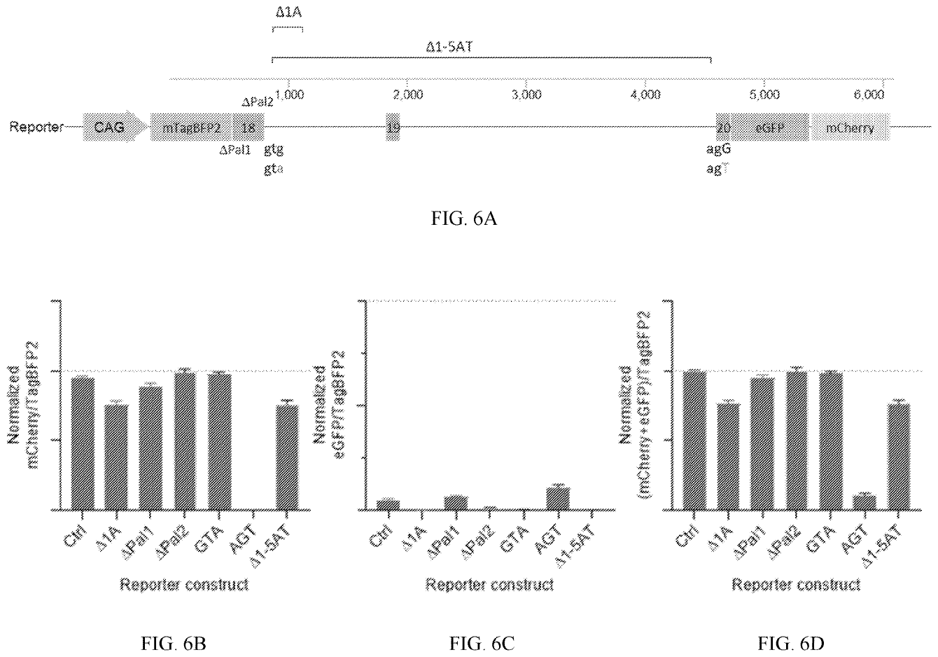

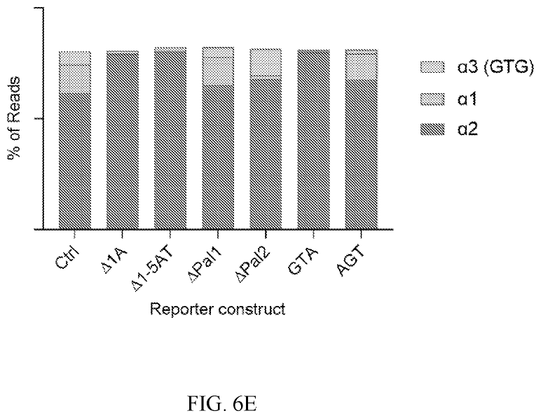

To study the molecular mechanism of α1 non-canonical splicing, base mutations were made at critical sites in the splice reporter. RNA secondary structure models predicted that palindromic sequences within exon 18 may support a hairpin structure. Therefore, mutations were introduced in each palindromic region (ΔPal1, ΔPal2), and surprisingly only the latter at the end of exon 18 (ΔPal2) eliminated α1 splicing. Another potential base involved in secondary structure at the beginning of intron 18 abolished GFP expression when mutated (GTG→GTA). A mutation at the beginning of exon 20 (agG→agT) did not decrease relative GFP expression, but abolished mCherry fluorescence, due to a premature stop codon introduced by the mutation. A large deletion of Δ1-5 (+13 to +3630) together with a deletion of a cryptic splice site shortly after the Δ5 region (Δ1-5 ag→tg; referred to in FIG. 6 as Δ1-5at) led to abolished GFP expression, but also a low (mCherry+eGFP)/TagBFP2 value, suggesting an increase in cryptic splicing or translation from non-spliced transcript that leads to TagBFP2 expression alone.

Amplicon sequencing with primers specific to the trichromatic splice reporter can be used to investigate splicing at single transcript resolution (FIG. 6E). mRNA was extracted, cDNA was synthesized through reverse transcription (RT) with a reporter-specific primer, and an amplicon was generated with reporter-specific primers including Illumina adapters. The results showed high concordance to the FL measurements, with the control reporter resulting in 76.6% α2, 15.8% α1 splicing. α3 splicing was also detected at levels (7.5%) similar to endogenous brain splicing (˜9%).

Deletion of Δ1A and mutation of the 3^{rd} base in intron 18 (GTG→GTA) both eliminated α3 as well as α1 splicing, confirming the fluorescence results and extending them to α3. Mutation of the palindromic region at the end of exon 18 (ΔPal2) reduced α1 but increased α3, demonstrating a dissociation between the expression levels of the two non-canonical splice products). A mutation at the beginning of exon 20 (agG→agT) did not change α1 but decreased α3. Amplicon sequencing proved advantageous here by directly reading out the splicing outcome rather than relying on a fluorescence measurement, which is misleading due to the premature stop codon formed by the mutation. The agG→agT mutation provided another important insight: the mutated ‘T’ was found in transcripts with α1-like splicing. Therefore, the missing ‘G’ in the α1 transcript in comparison to the α2 transcript is likely the last base at the end of exon 18 rather than the first of exon 20.

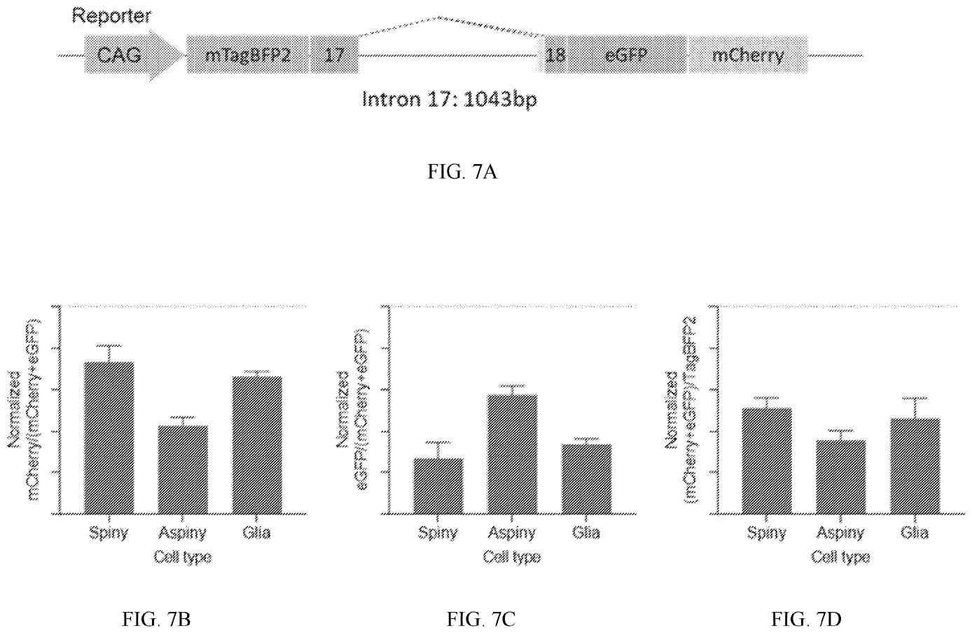

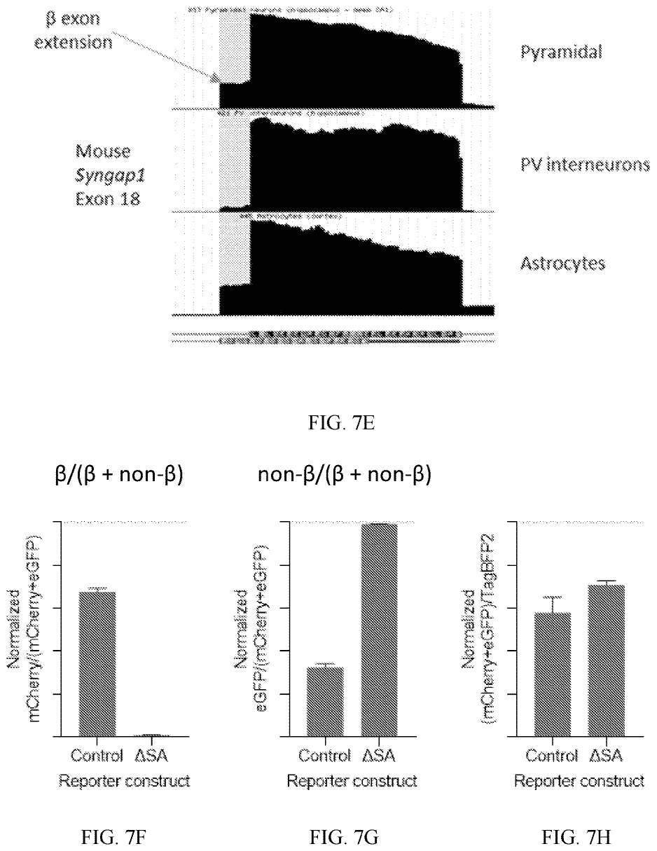

Another trichromatic splice reporter was designed to interrogate the β/non-β splice decision between exon 17 and the exon 18 extension (FIG. 7A). This reporter construct fluorescently reports the relative expression of SYNGAP1 β (mCherry) and non-β (eGFP) and through the frameshift arising from alternative splicing. Total SYNGAP1 minigene expression is reported by mTagBFP2 expression which is in all minigene transcripts. Primary cultured rat hippocampal cells were transfected with the β/non-β trichromatic splice reporter construct. Aspiny neurons (based on eGFP morphology), likely GABAergic interneurons, display lower mCherry and higher eGFP ratios compared to spiny pyramidal neurons and glial cells. Single-cell RNA-seq data shows lower β usage in PV+ interneurons in comparison to (spiny) pyramidal neurons and astrocytes (glial, FIG. 7E), consistent with the results from the β splice reporter (FIG. 7B). Based on coefficients estimated from the c-terminal reporter (FIG. 3C), the values of (mCherry+eGFP)/TagBFP2 are lower than unity, suggesting the existence of splicing outcomes other than the expected β and non-β.

Additional splice reporter constructs including a tetrachromatic α1/α2/γ splice reporter that adds a far-red FL to report γ splicing were generated and validated for the simultaneous measurement of 3 splicing outcomes. To facilitate large scale genetic modifier screens in ES/iPS-derived neurons, a piggyBac transposon-based trichromatic α1/α2 splice reporter was generated. The splice reporter was flanked by ITRs that allow the stable integration of the entire reporter to transfected cell genomes in the presence of PB transposase. This reporter was generated with a deletion of the Δ2 region (which includes the γ exon) to focus on the α1 vs. α2 splice decision. The TagBFP2 coding sequence was replaced with emiRFP670 to confer compatibility with BFP-expressing cell lines. The list of currently available human SYNGAP1 splice reporters therefore includes:
Trichromatic α1/α2 splice reporter (TagBFP2/eGFP/mCherry)
   Δ1, Δ1Acon, ΔPal2, GTA as ‘no α1’ positive control
   Δ2 as ‘no γ’ positive control.
Tetrachromatic α1/α2/γ splice reporter (TagBFP2/eGFP/mCherry/emiRFP670)
   ΔPal2 mutant as ‘no α1’ positive control
   ‘ΔPal2/Δγ splice acceptor’ mutant as ‘no α1/γ’ positive control
   ‘ΔeGFP and mCherry coding sequence’ deletion for ‘no α1/α2’ positive control
   ‘short’ version with no α2-specific CDS nor 3′UTR
   ‘full’ version with α2 CDS and human 3′UTR after mCherry CDS
   mCherry coding sequence′ deletion for ‘no α1/α2’ positive control
piggyBAC trichromatic α1/α2/γ splice reporter (Δ2) (emiRFP670/eGFP/mCherry)
   ΔPal2 mutant as ‘no α1’ positive control
Trichromatic β/non-β splice reporter (TagBFP2/mCherry/eGFP)
   Δβ-splice acceptor mutant as ‘no β’ positive control

### Anti-Sense Oligonucleotides (ASOs) for SYNGAP1 Haploinsufficiency Treatment

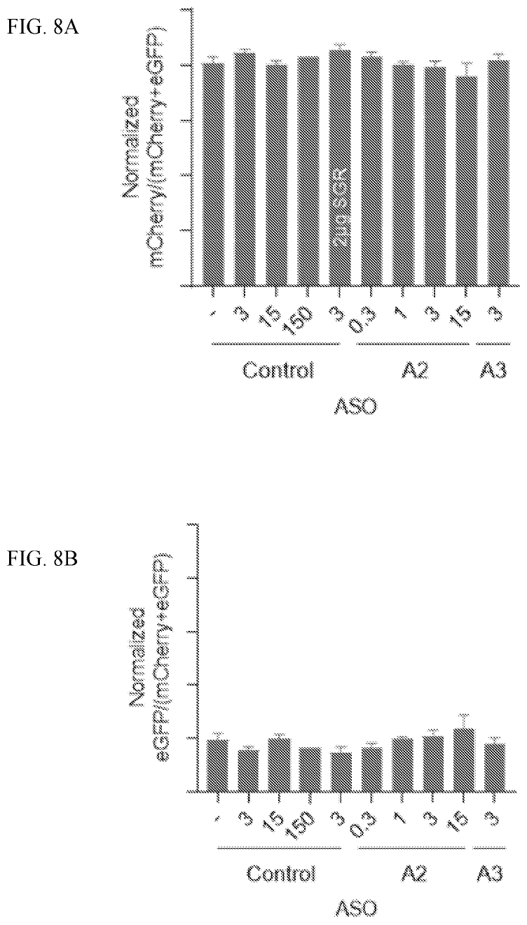

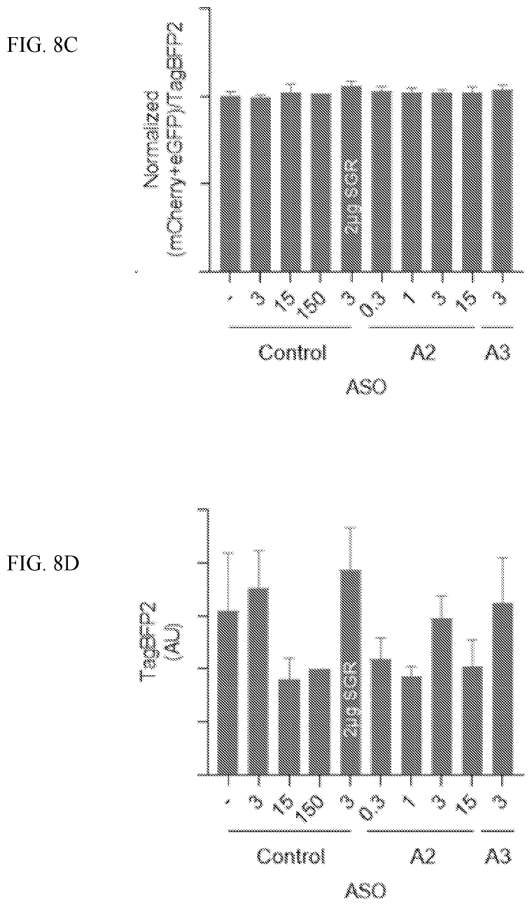

15-20 bp ASOs tiling critical splice-modifying sequences were screened in focused ASO ‘walks’ within SYNGAP1 with the goal of increasing SynGAP-α1 expression. A titration experiment was first conducted to determine the dose of ASO treatment on rat hippocampal neurons and the ratio to the co-transfected splice reporter plasmid that would allow sufficient FL expression (FIG. 8). High molar excesses of the ASOs led to lower transfection rates and TagBFP2 FL expression levels, while ratios remained stable. 2 μg of splice reporter plasmid and 15 pmol ASO per well were co-transfected into 12 well plate cover slips of cultured neurons in each of the following experiments.

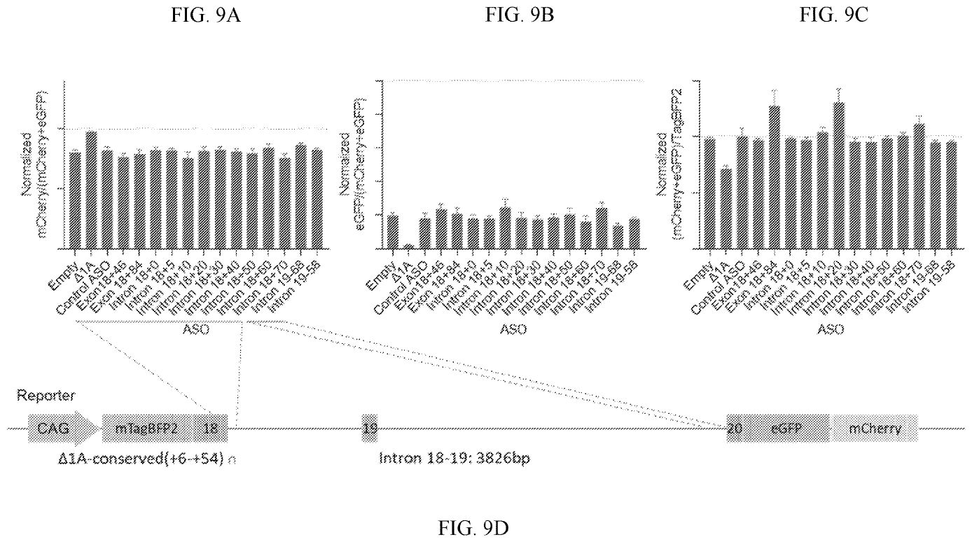

An SSO screen of 14 ASOs spaced at 5-10 bp near the Δ1A-conserved region as well as some targeting exon 18 and at the end of intron 19 was performed (FIG. 9). The Δ1A positive control showed lower eGFP/(mCherry+eGFP) levels (F_{15,101}=4.833, p<0.0001, 1 way ANOVA; p<0.0001 for Control ASO vs. Δ1A, Dunnett's multiple comparisons test), however all other groups did not display statistically different levels (p>0.05 for Control ASO vs. all other groups). The (mCherry+eGFP)/TagBFP2 values were significantly increased with the treatment of ASOs Exon 18+84 and Intron 18+20 (F_{15,101}=6.701, p<0.0001, 1 way ANOVA; p=0.0157 and 0.0008 for Control ASO vs. Exon 18+84 and Intron 18+20, respectively, Dunnett's multiple comparisons test). This suggests a suppressive effect on γ splicing that leads to a reduction of TagBFP2-only expression, thus increasing the value of (mCherry+eGFP)/TagBFP2. These results demonstrate that the splice reporter can be used to efficiently screen ASOs that modulate SYNGAP1 splicing.

Together, these results demonstrate that chromatic splice reporters can be used to identify agents that can modulate SYNGAP1 splicing (e.g., SSOs that can suppress expression of a SynGAP1-α2 polypeptide, a SynGAP1-α3 polypeptide, a SynGAP1-β polypeptide, and/or a SynGAP1-γ polypeptide, and/or can increase expression of a SynGAP1-α1 polypeptide and/or a SynGAP1-α2 polypeptide) that can be used to treat diseases and disorders associated with SYNGAP1 haploinsufficiency.

### Example 2: Modulation of SYNGAP1 Non-Canonical Splicing and Development of ASOs for SYNGAP1 Haploinsufficiency Treatment and Other SynGAP-Related Developmental Disorders

The results in this Example re-present and expand on at least some of the results provided in other Examples.

### Targeted Deletions of Intronic and Exonic Regions Identify a Core Region Essential for α1 Splicing

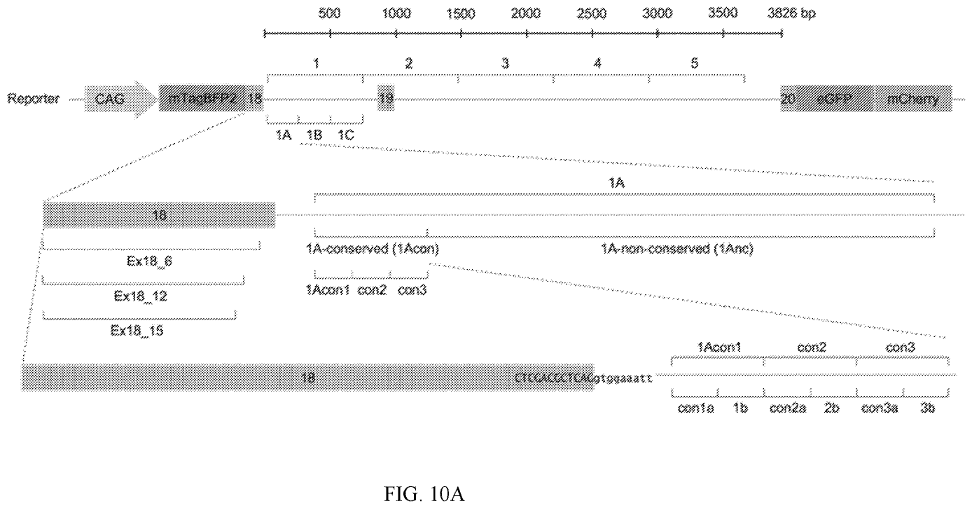

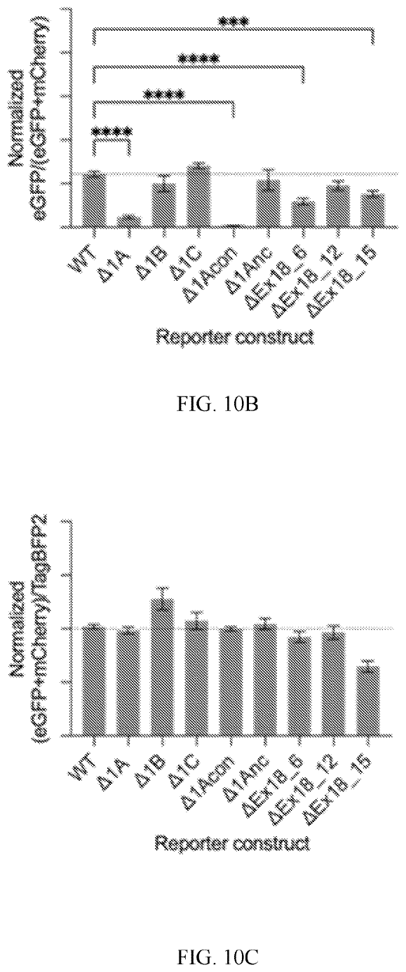

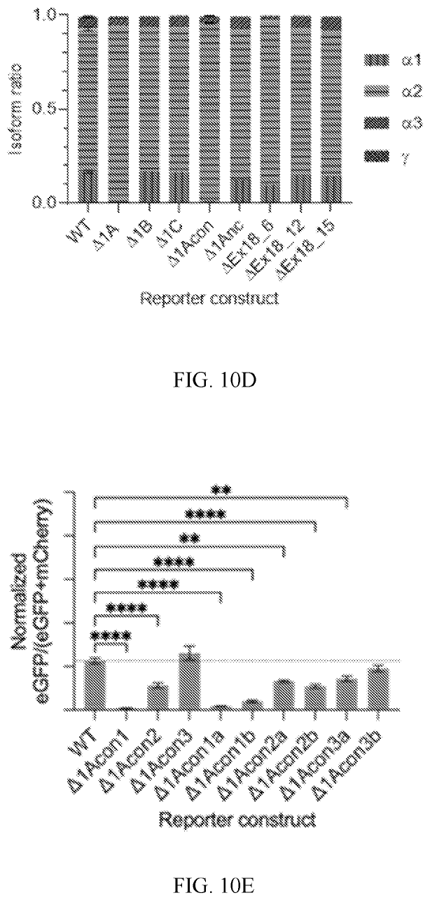

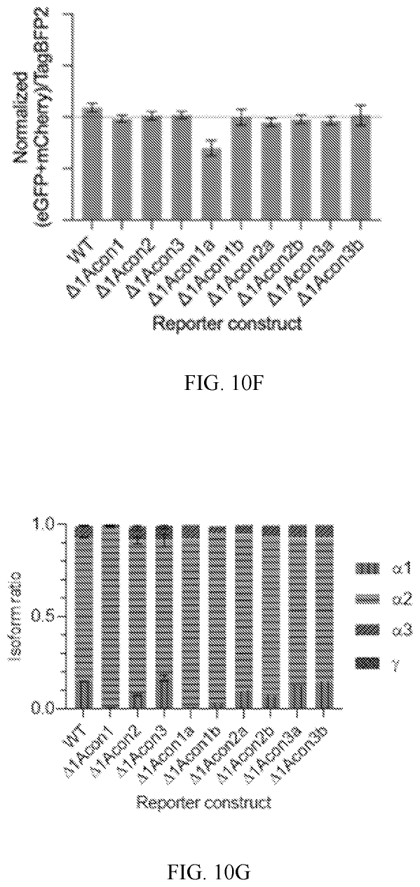

Further deletions within the evolutionarily conserved region 1Acon revealed a 14-basepair (bp) region 1Acon1 critical in SYNGAP1-α1 splicing (FIG. 10). The region 1Acon2 facilitates α1 splicing. Removing all of exon 18 except the last 6 bp (ΔEx18_6), 12 bp (ΔEx18_12), or 15 bp (ΔEx18_15) revealed the 5′ portion of exon 18 may have a minor modulatory role on α1 splicing.

### Specific Mutations and Deletions Identify Nucleotides Essential for α1 Splicing

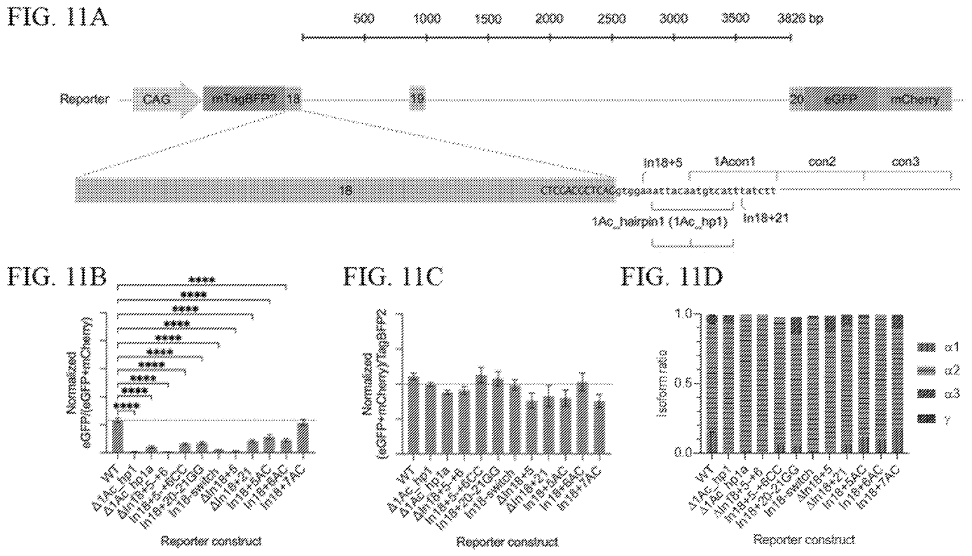

Deletions or mutations of adenosine nucleotides within the 1Acon1 region (ΔIn18+5, +6, +7) lead to substantial decrease of α1 splicing (FIG. 11). These results identified critical motifs and nucleotides that support the non-canonical splicing.

### Manipulations of the Splice Donor Site Disrupted α1 Splicing and Induced the Use of an Alternative Donor Site

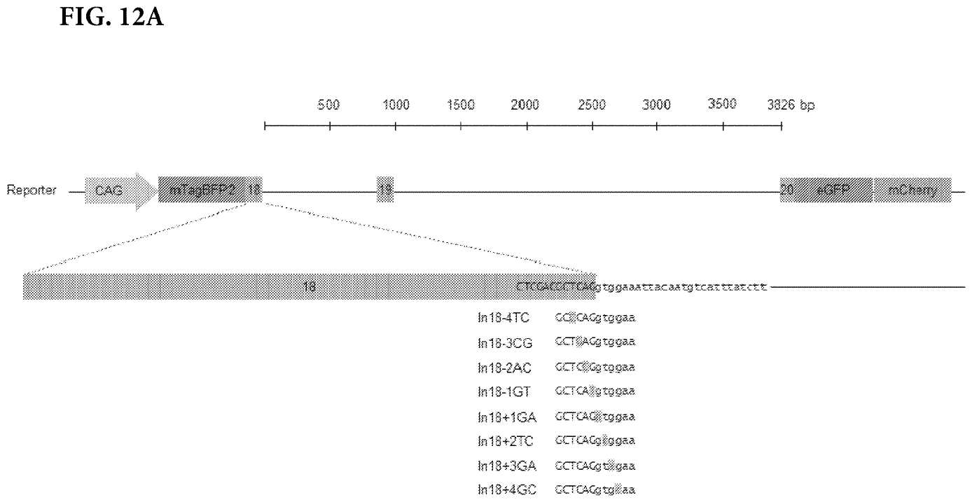

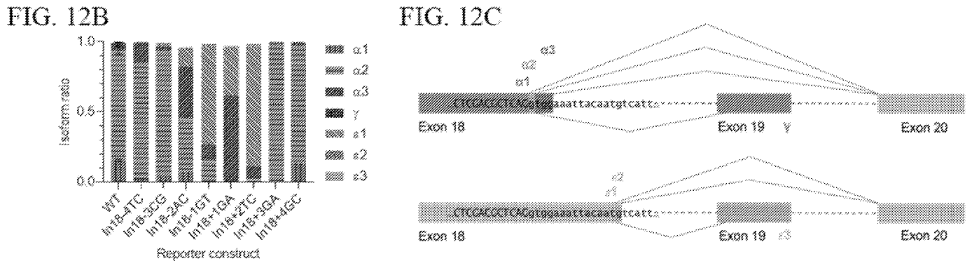

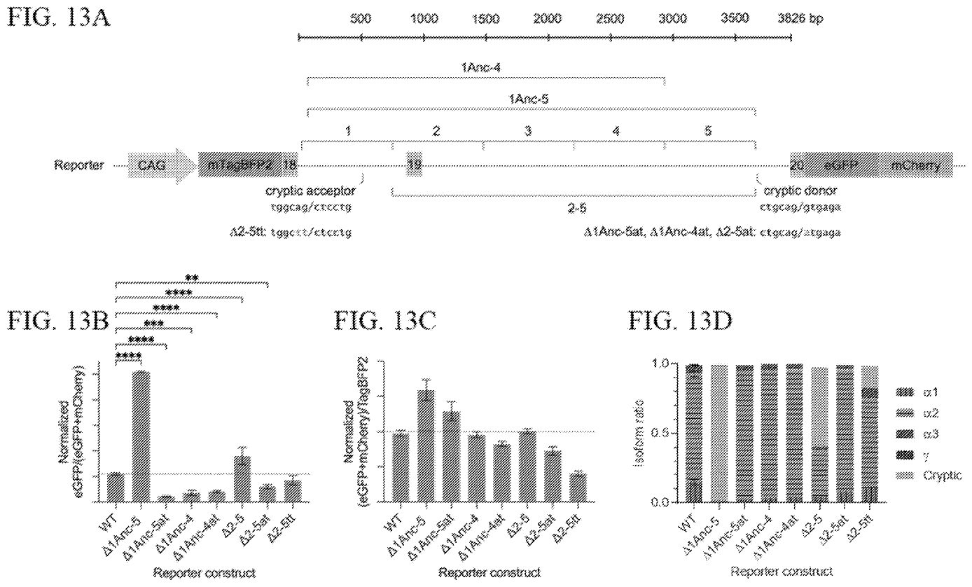

Mutations of the eight nucleotides around the splice donor site of exon 18 revealed that all splice donor mutations except In18+4GC reduced α1 splicing (FIG. 12). In addition to disrupting α1 splicing, mutations of the four nucleotides immediately around the splice junction of exon 18 (In18−2AC, In18−1GT, In18+1GA, and In18+2TC) induced the use of an alternative donor site 14 bp downstream of the α2 splice site (FIG. 12C). Surprisingly, this alternative donor site produced a splicing pattern strikingly similar to that of the original site. It gave rise to three splice variants, which were named ε1, ε2, and ε3. Like α2 and α1, ε1 (canonical) and ε2 (non-canonical) differ by 1 bp at the 5′ end and both splice to exon 20. The variant ε3 behaves like γ in which it shares the same 5′ splice site as ε1 but splices to exon 19 instead. Although α1 and ε2 are both produced by non-canonical splicing, they are different in two ways. First, α1 splices 1 bp upstream of α2, whereas ε2 splices 1 bp downstream of ε1. Second, α1 is produced by GG/AG splicing, while ε2 is generated by TC/AG splicing. In the samples with noticeable use of the alternative donor site, ε1 was the dominant cryptic form. Aside from reducing α1 splicing and inducing cryptic splicing, In18−2AC and In18+1GA also increased the ratio of α3 reads compared to WT (WT: 0.0547±0.0183 vs. In18−2AC: 0.372; In18+1GA: 0.617) (FIG. 12B).

These results show that the SYNGAP1 exon 18 to 20 junction can support non-canonical splicing at more than one site and donor motif.

### Large Deletions Disrupted α1 Splicing and Triggered Cryptic Splicing

Large deletions involving multiple regions (FIG. 6A) showed that the genomic regions between exon 18 and exon 20 are cooperatively involved in α1 splicing such that large (>2500 bp) deletions disrupt α1 splicing and trigger the use of cryptic donor and acceptor sites.

### Anti-Sense Oligonucleotides (ASOs) for SYNGAP1 Haploinsufficiency Treatment

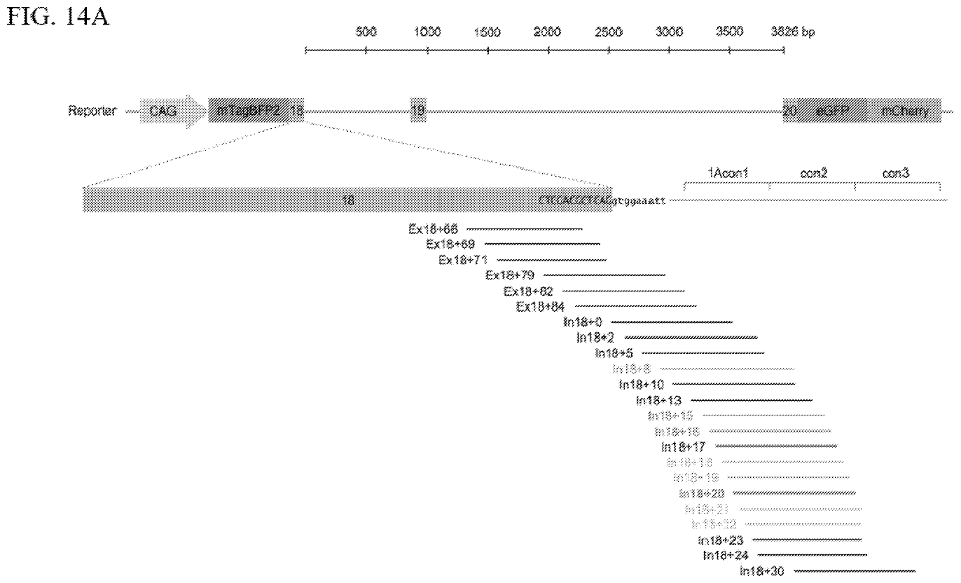

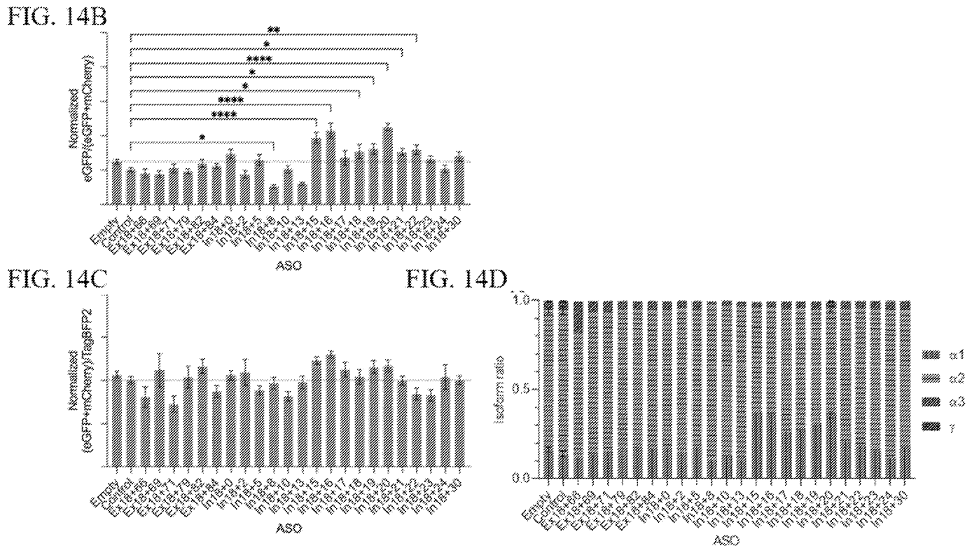

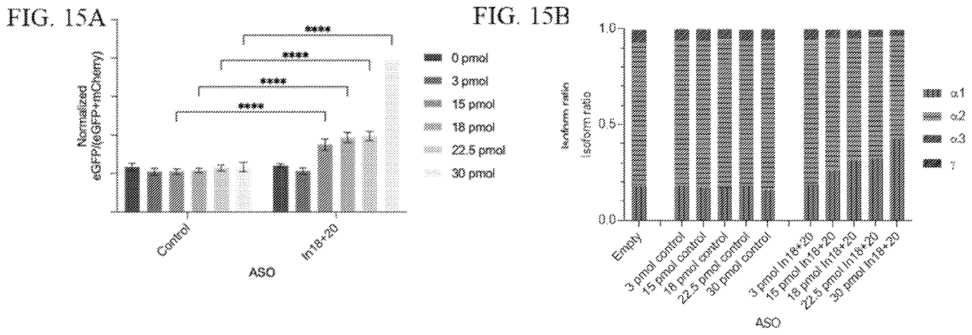

15-22 bp ASOs tiling critical splice-modifying sequences were screened in focused ASO ‘walks’ within SYNGAP1 with the goal of increasing SynGAP-α1 expression (FIG. 14A). The walk highlighted three ASOs with significant positive effects (dark green; Control: 0.202±0.0110 vs. In18+15:0.386±0.0314, p<0.0001; In18+16:0.4286±0.0439, p<0.0001; In18+20:0.449±0.0210, p<0.0001), four ASOs with moderate positive effects (light green; In18+18:0.307±0.0410, p<0.05; In18+19:0.323±0.0304, p<0.05; In18+21:0.305±0.0204, p<0.05; In18+22:0.318±9.0273, p<0.01), and one ASO with significant negative effects (red; In18+8:0.104±0.0101, p<0.05) on α1 splicing compared to the negative control ASO (Control) (FIGS. 14A-D). Some ASOs also had a large impact on ratios of α1 reads based on amplicon sequencing but failed to reach statistical significance with their fluorescence results. These results demonstrate SYNGAP1 c-terminal splicing can be effectively modulated with ASOs.

### A Lead ASO Induced a Dose-Dependent Increase of α1 Splicing

To evaluate the dose dependency of In18+20, 3, 15, 18, 22.5, and 30 pmole of control and In18+20 ASO were co-transfected with the WT reporter. A two-way ANOVA was conducted on the normalized eGFP/(eGFP+mCherry) ratios to determine the effect of ASO (control vs. In18+20) and dose (amount of ASO transfected) (FIG. 14A). A statistically significant interaction (F (5, 134)=11.25, p<0.0001) was revealed, suggesting a dose-dependent effect of In18+20. Post hoc multiple comparison suggested that the effect of In18+20 was significantly different from that of the control with 15 pmole and higher doses (p<0.0001). Quantification of the ratio of α1 reads in amplicon sequencing corroborated the conclusion about dose dependency of In18+20 (FIG. 14B). Therefore, the In18+20 ASO substantially increases the ratio of α1 splicing from the SYNGAP1 splice reporter and is a promising candidate for further optimization and evaluation.

### Example 3: Diseases and Disorders Associated with SYNGAP1 Haploinsufficiency

ASOs that target additional SYNGAP1 genetic variations are screened for the ability to modulate SYNGAP1 splicing (e.g., to act as an SSO that can suppress expression of a SynGAP1-α2 polypeptide, a SynGAP1-α3 polypeptide, a SynGAP1-β polypeptide, and/or a SynGAP1-γ polypeptide, and/or can increase expression of a SynGAP1-α1 polypeptide and/or a SynGAP1-α2 polypeptide). For example, ASOs that target SYNGAP1 genetic variations shown in Table 5 are screened for the ability to act as SSOs. For example, ASOs that target SYNGAP1 genetic variations described elsewhere (e.g., Vlaskamp et al., Neurology, 8; 92(2):e96-e107 (2019); Bowling et al., Genome Med., 30; 9(1):43 (2017); Hamdan et al., Am. J. Hum. Genet., 101(5):664-685 (2017); and Gieldon et al., PLOS One,; 13(8):e0201041 (2018)) are screened for the ability to act as SSOs.

Due to the high penetrance of heterozygous SYNGAP1 mutations for epilepsy, intellectual disability, and neurodevelopmental delay, human SYNGAP1 variation informs considerably on the structure-function relationship as well support the proposed therapeutic strategy. For instance, human SYNGAP1 disease variants have revealed that loss-of-function mutations in exons 1-3 are less severe than mutations beyond exon 4 (Mignot et al., J. Med. Genet., 53 (8): 511-22 (2016); and Vlaskamp et al., Neurology, 92(2):e96-e107 (2019)), likely due to alternative TSS sites between exon 3 and 4 and beyond.

On the c-terminal side of SYNGAP1, several disease variants have been noted in exon 17-20 that lead to premature termination and potentially NMD. Of these, c.3826dup leads to a frameshift and premature termination of non-β isoforms at the stop codon of the β isoform. β-spliced transcripts instead get elongated but include frameshifted exon 18 sequences. This disrupts the coiled-coil domain (which is critical for trimerization and interaction with PSD-95) in all c-terminal isoforms, leading to an ‘all-β-like’ scenario. The symptoms of this patient confirm that β-like c-termini are not sufficient for normal neurodevelopment and mental function, suggesting the non-β isoforms are critical.

The splice acceptor for all non-β isoforms is mutated in the two reports of c.3795-1G>A variant carriers in Clinvar diagnosed as SRID/MRD5, which would likely lead to large loss of non-β isoform expression. A nonsense mutation c.3811G>T, which would lead to truncated non-β c-terminal isoforms, has a carrier in Clinvar reported as SRID/MRD5. These human variants confirm that the loss of non-β c-terminal isoforms of SYNGAP1 leads to SRID/MRD5.

TABLE 5
Human genetic variation at the end of SYNGAP1. Note that first four variants likely
recruit significant NMD, confounding interpretation. The last mutation is unique
to α2, but the carrier displayed mild symptoms (focal epilepsy, no ID).

| Allele Change | Residue Change | Variant type | Inheritance | Family type |
|---|---|---|---|---|
| c.3657T > G | p.Tyr1219Ter | stop_gained | De novo | Multi-gen |
| c.3718C > T | p.Arg1240Ter | stop_gained | De novo | E-Multiplex |
| c.3740_3746del | p.Ile1247SerfsTer2 | frameshift_variant | De novo | — |
| c.3748C > T | p.Gln1250Ter | stop_gained | De novo | Simplex |
| c.3795-1G > A | | splice_acceptor | De novo | c.3795-1G > A |
| c.3795-1G > A | | splice_acceptor | Unknown | c.3795-1G > A |
| c.3811G > T | p.Glu1271Ter | stop_gained | Unknown | c.3811G > T |
| c.3826dup | p.Asp 1276GlyfsTer7 | frameshift_variant | De novo | — |
| c.3834dup | p.Ala1279fs | frameshift_variant | Unknown | |
| c.3959C > A | p.Pro 1320His | missense_variant | Unknown | Multi-gen |

### Example 4: Exemplary Sequences

An exemplary exon 17-18 minigene sequence
SEQ ID NO: 1
AGCAAAATCCTGATGCAGTATCAGGCCCGACTGGAGCAGAGTGAGAAGAGGCTAAGGCAGCA
GCAGGCAGAGAAGGATTCCCAGATCAAGAGCATCATTGGCAGGTGAGGGGCGGCctggggag
ggggttgtgagggagagcctgaggctggagagagcaagtgggcgagctactcctctgactcc
catccccaaactcaggagcccaccaggagagcccaccactctcctccccaggaagccaccca
ctcactcatcaccagatggagagaaaccccaacctgcttagtgcattaaatatctctttacc
aaaccctgacctctcttctgatagagtagcttcggaagcccttggaaaatgtacctgttcct
gtccaaccatcactgcatttgcatttaccctaggccagagctccccactagttattctcaac
ttaacctgtgatgttcactccaaacctaagcagggctccctagccagagtaggccctgccct
tcctgggtggaccctccctctctagccttggaaaggtgttctgttagaaagggtcttttagc
ctgtgtatgttttcagctgctccagcaagtcctgggctccaaagagggtatcctcagcaaag
aggtcaattatcttcagagtggtggggtcggggtgggggggaccctgggcgcactccaacca
gagccacctccattttgatccattctaaatgtattttatgtaagatttaattagaagaaaag
ggcttcttgaaatattttttgaaaaccactgctctaattgatatcctttatgataaatcacc
tcgaggatcttcacagtgaggtgacatgggggatgcagaaggcaggtcctcagccatggaag
gtctggggaaggggcactgctgtcctgattgggacgatggaggcctggaggtgtctggatgg
tagaagtctttgaggcacagaaagctgccttagcagggaggtgtaagggttcctgggaggaa
ggtggagagcatgatcctgaggaacagggagtcttgcatcacggcaatggagggactctgat
tctaagGGATAAGGATGCCTGAGGTTTTTCcagagagctatggggttccatgggcaggctct
gagcctgtgcccgccactaaccccactgaagCCCGTCCCTT
An exemplary exon 10-11 minigene sequence
SEQ ID NO: 2
GAGAATTCATCCGTGCTCTGTATGAATCTGAGGAAAACTGCGAGGTAGACCCTATCAAGTGC
ACAGCATCCAGTTTGGCAGAGCACCAGGCCAACCTGCGAATGTGCTGTGAGTTGGCCCTGTG
CAAGGTGGTCAACTCCCACTGGTGAGACTGGGAACGCTGGGCTGGGGGGCCAGGGTCGGGGG
AATTATGTGTTCATCTGTTCATCTATCTGTCCATCCTCAAAGAGGactgagcaccatttatg
ggcaaagcattgttctaggcgctatagagcaaacaggtgaaagaggcctggtccctgccctc
agagggcctccaccagaatggggacaaattagaagaaaaaaaaaaaaagccacagagccata
atggtgtgtaagtgctGAGTAAGGGTCCCCCCAACCTCTGTGTGACATAAGGTCAGAGAGAA
GGCAGAGCTTTGAGATAAGTGGGGAAGAGGTGCCCCCTTGGGTAGGCTTTGAAGACTGGTTT
AGGTTCTGATATATGGACATAGTTGGCAAGAAAGACATTTCAGAAGAAGGCTGTGAGAAAGG
CACATGTGTGATGGTGAAAAGGCCCAGGAGTTTTCAGGGGACATTAAAGTAGGTTAGTAGCA
ATTACATCAGGTTTAGTGGAGCATGTGCCTCATAATGGGGAGTGGCGGGAGAGATGTCTGGG
CAGGAAGATTAGCTTTAGAAACTGGAAGGCCTCAAGGAGTCTGAGGTCATTGGTAGGCCTTG
GGATGCCATTAAAGGTGTCAGAAATATTGTGATTTAGAAGATTAATCCATAggctgggcacg
gtggctcacacctgtaataccagcactttgggaggctgagggggcagaccacctgaggtca
ggagtttgagaccaggctgaccaacatggagaaaccctgtctctactaaaaatacaaaatta
gccagatgtggtggcacatgcctgtaatcccagctattcgggaggctaaggcaggagaatca
cttgaatctgggaggtggaggttgtagtgagccgagatcacacgattgcactctagcctggg
caacaagagtgaaactccatctcaaaaaaaaaaaaTTAATCCATAAGTAGAGTGTATGTAGT
AGAGTAGAGGGATGTATGTTGGGGGATGACTGGAATAGAGCCAGCTGGGGGCTCGGAGGCAT
GAAAGTCAGATcctgaatcagaacagtgacagaagttaggaaggagctactggaaggaccct
ctcgggaaaggatcagtaggaattgtcagcttattggaaaggagggagcatgtctgtgggga
gtcacggatgactcaagaggccatgaggctggtggttgggagaccggtggcctcattgacaa
ccaggaaagtcaggaggaggagccagttggaggtgtgggggcgatggtgagctctgctttac
accagccgagtttaaggtgtcagtgggacattgaagtggaactgGGAGGTGCGGGGAGTGAG
CTCTGAGCCATTCCAGGGACTGGGGATCATGCCTGGGGCACCTCCATCCCCATTTCCCTGGA
ATCCAGAAGAGTTGGGGGGTCCGAGCTCCCTGTACCTCAAGTGACCCTccatctctctccca
tctctgtctctccctggtgtctgtttttcttctcctcctctccttgtctctctcccacaccc
ctccatctctctcccacgtgtctctcccctcaccttctctccccctccatttctctctccct
aatctgtctgttccctctGCCATGGCCCCCTTCTTCAAGCAGCCTCCCATCTTGCTCCTGCG
GTCCCTCCTTCCCTGTCTCTCTCACCCCTGTTTCCACACCCTCACCTCCTACCACCCCCCTC
AGCATGTTCCCTGGAAGCTGAGGGTCTCTGGGGCTCAGTCCCGGtctctctctttctctctc
tctctctctgtctcCCCGACCCTTCCCCCCAGCGTGTTCCCGAGGGAGCTGAAGGAGGTGTT
TGCTTCGTGGCGGCTGCGCTGCGCAGAGCGAGGCCGGGAGGACATCGCAGACAGGCTTATCA
GCGCCTCACTCTTCCTGCGCTTCCTCTGCCCAGCGATTATGTCGCCCAGTCTCTTTGGGCTT
ATGCAGGAGTACCCAGATGAGCAGACCTCACGAACCCTCACCCTCATTGCCAAGGTCATCCA
GAACCTGGCCAACTTTTCCAA
An exemplary exon 18-19-20 minigene
SEQ ID NO: 3
ATGCTGGTGGAGGAGGAGCTGCGCCGGGACCACCCCGCCATGGCTGAGCCGCTGCCaGAaCC
CAAGAAGAGGCTGCTCGACGCTCAGGTGGAAATTACAATGTCATTTATCTTCTCCGTGTCCC
ATCCCCATCCATCCCACTGTCTTTCGTGCACTCACTACACCAGCCACCTAGCCCCATCACCA
TCTGTCTCTCATAGTCTGCTGTTTGTCCACTGGCTGCTCCTGGCAGCCCCCTAGTGACCCCA
TCTTCATCCCATCGTCTGTGCCTTTGTCACTCCTGGCAGCGTCAGCCCAACTCCTGTGCCTT
CCCATCCAGTCTTCCCACTCCTCTCTGCATCTCAGGACCTTCTCTACCAGAACCTTGGTCTT
TCTGCCCCTAGACCCCACCTAGTTCCAAGAACCCCTGCCCCTTCTTTGCTCACTCCTATTCA
AGCCACGTTGTTCAGCTTCCTCTGCGCTCTTGGGCCAGAGGGCTAGAAGCTGCCGTTTTCTG
GAATAGAGCACAGGGCAGTATGATCTGTAGTTTCTCCAGGCCCTGGCCGGTACCCTGAAAAC
TTGGGGACCCATCACCTCTGTTCTCTTGGCTCCCTAATTTTCCTGTCTCCTTGGCAGCTCCT
GCATAGCTTCCTCTTCCTGACTCTTCAGATCTTGAAGGCCTTCCATCCTGTAACCTCCCTTT
GCCCTCAGTATTTAAGTCCAGCCTCCCTCTGGCCTCCCTCCCACTCTGGCCCTCAGACCTTC
CCAGCTGCCTGCTGCCCAGCCTCTCTTCTCACAAGCCAGCTTCTAGGACCTCCCTTCTGCAC
CCTTACCCCTTGCTTTCCCAAAATTCTGCTCATTTTCCTACCCATACTCCTCTTTGCTCTGA
CTGCTAGGCTCCCCCCGCCTGCCATCCCCCCACCAAGGCTCCTGACCCCATGACCCCACTCT
CTCCCACTGCAGCTCCTCATCAGGTAATTCTCCTGGTTCCGCTTTGGCCACGGGCGGAGGAC
ACAGGGGGAGGTGACTCCGGACCACTGCAGGTTGGTCGTGAAGCCCACTCCCTCCAACACCT
CCGGAGCCTCTCCCCTCTCACTGCTGCCCTCCACACCCAGAGAACCTCCACAGACTCCAGCC
CTCCGACACCTGCACAGATCCATCTCCCAAGACACCACCCAAAGAGAGCATTTGCTGCTGCT
TCCCAGAACTGTCCAACAATACCTTAGCAACACCAAGAGTTGGGCCCTAGATGGGCCCAGCA
CATTCACAGGTCACACCCACTTCCCTGCAAAACCCACCCCCTCCCAGCCTCCTCCTGACTCT
AAGCCCTCCTCTTCCTCTACCTCTCCAGTGTATGTCTGTCACCCCCCATTTCACCAGAGCGT
CCTTAGGGGCTGGGGGTGGGTTTGTTAATGGGGTGGAGGCAATGATGGGTTGGAGGATCTTG
GCTATAGGGGCTGTGCTGACTGCAGCAGGTAGGTTGGGTTTCCCTCTTCCTTCCCTAATCTT
GGTTCTCTACCCTCCTTTCCACTCCTCACCTGATTCTCTCTCTTCCTCCTCCTTATATCTGT
GAGGCAGAAGGCATCTGAAGCTCATATTAGCCCCCATTGGGTGGGAATTAGGAGTGGGTAGT
TAACTCAGGGAGACTTGAGATACCCTGGAAAAAATGCTATTGAGATGTCCTGACATTAGGCA
GGGTGGATGGAACAAGAAGGAGCAAGAAAGGAACCTCAGGCAGATGTTAGGACATGGACTTG
ATCATGTGGCCTGGGAGTTTAGAAATGGGGAGAGACATCCTCCTAGATCAGATCGTGGGCTC
AGTAGGCATGTTGATTCCCAGGGAGAGGTGCCAGGAACAGCATGGTAAAGAATGTACTCTTC
ACAGCTCACATCCCCAGGTTGCTGATGCCACTCACTCCCCCTCTCCTGCCATCGAGTGGCCT
TGCCGGACACATCACCCTACCTAAAAAGCCAGTAAATGAGAACCTGTCAGCTATAGCCATCA
TTTCTGAGATGCGATTTTCTTTGGGATTGAGCTGCAGTGGGCAGTGGCTCCTTACACTGTAA
TTTTAATTCTCTGCCTGCCCAGCCTCTCTGTCAAAGTAGCTGGTGATCTATAAAGATGCTAA
AAGGCACCAGGGGACTTTGCCATTTAAAGGACTCCTGCAGTGAATTCTTTTGTAAAATGAAT
AATGGCACCCTAATTTATCCACTTTCTAAATTTGGGTCCATGGGGGTGTCCAGGGCATGCTT
ATGTGCTGTCACCAGCAGACAAACAGAGGGAATGGAATCTGGGGGTTCCTTCCCTGCTCTCC
CGCCATACTCAGGATACCCTACCATAAGTGATTTCCTCTCACTGACTTGCAGAAAATGTGTG
AGATACCCAGCAAGCTAAGAAGGCAGTTTTGCTGGGTATCTCATACCCAAGGCTGGGGTTTG
GGTGATCTGAGAGGTTAGCTCCTTGATCCTAGGATGGAAGGGAGAGCTTATATAGAAGCTTT
TACTTGGAAGGTTTTGTATCCTAAGGTCAGACATAGCTATATTACCAAGCCTAAATGCCATG
TGGCCCAGGAAATAATTTGGACATTTGTTCTAAACCACTTGTGGTAGGTATTGGTCTCTCTG
CAACTCAGCCATTAATTAGAAATTAGTTTTGAGCCTGAATTTTAAAAAGCCAAGTGTTGCCC
CCAGCCCACACACACACACACGGACATGTACAGTACAAACCCCAGATAATTACAACAGCCAA
AGAGAGAAGGAAGTGAATTTCCCAACCAGAAGCGTAGGGAAATTCAGATGGCTTTCTTTTCT
CCCAGCAGAGGAACAGAAGGCGGAGCTAAGGGCAGGAACCAGGAGTTGGTCAAGGAGCTATA
GGAGGTGATGAGAGTAGAACCAGGGGTAGGAGCTGGTCTGGTACCCCTCACCCTCTAATTGG
GAGCCCAGGGAGAAGGACTGAAAAGAAGATGGGAGTGGAAAAGAATAAAGCCAGTTTCTGCT
TCCCAGGGATGCAGAGATTGGGGCATGCTGTGTCTGCAGAAGCTCCTAGTCATTTCCGCCAT
AATTGTGAGAGAGAGGGGCAGCCCTCCCACAAGATTTTTCCCTTCCCATATCACTTCCCTGA
ATCCCCTTCCTTTCCCCCCAGTACAGTTAAACCTCTCTCTAATTTGGAATGTTATATTTGAG
AAGATGGCCACTGTGAATAAGTGACAAACGGAATGACAGTGTCTATTTAATGAAATGCATGT
CTTCAAAATATATACAGAACTCGATGAACAAGGCTTTTTCCACTCCTCAGGGAGCATGCATT
AATGAATAGATAGGATTCAAAAGTCTGTTTTCTGGTATGGGTTAAATATCCCCTCCTACAGA
CATATTTCCACCACTAATTTGCTTAGTACACCTTTTCTTCACAGATAAAGGAAAATGCAAGC
TCAGTTTTTCTTCAGATTATGAAGAAATTCCAAATCCACAGGGGTTTGGATTAATGAGGTTT
TGCTGTACTGCCTCCCCTTATTCCTCAACATGAAGTTCCCACCTCGGATTGGGGATGGGTGG
GAGGGGGTTTCAAGAGGAGGAGGGTGGGATGGGCAAGGAATATACACAGGTGAAGCCAGAGA
AGGGTTAGGTTGGGGGTGCGGTGGGAACTTGCTGTTTTGATCTGGTTTCCTGGTGTGACACT
CTGGGTTAAAGGCTTGAAGGCCCCTGTTAGGAGTCTAGGGGTGAGATTCTCTTCTCTCTGAT
CCCAGAGGACGTTAACTTCTACTGCAGGTGAGAAACAAAATAGGAGGATGGTGGGGACTGTC
CTGGGAGGAGGGGGTGGTCCATGGCTTGTGGTGTGGGCTGGCTATAGGGGAGGCCACTGCTA
GGGGACTGGCATCCAGGCCCCCTTGAAGCGTCTCAATAAGTCCGCGCTCTCCTTTTTGGTGT
CTTGCAGGAGAGGCAGCTTCCCCCCTTGGGTCCAACAAACCCGC

### Example 5: Treating SYNGAP1-Related Developmental Disorders

A human identified as having SYNGAP1-associated NDD (e.g., SRID) is administered (e.g., by ICV injection) one or more SSOs that can modulate SYNGAP1 splicing described herein. The administered one or more SSOs that can modulate SYNGAP1 splicing described herein increase the level of the Syngap1-α1 polypeptide within neurons in the human.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A nucleic acid construct for identifying a SYNGAP1 splice event, said nucleic acid construct comprising:
(a) a promoter sequence operably linked to a nucleic acid encoding a first reporter polypeptide;
(b) a SYNGAP1 minigene comprising (i) at least two exons and intervening intron(s), (ii) a first splice donor/acceptor pair, and (iii) a second splice donor/acceptor pair;
(c) nucleic acid encoding a second reporter polypeptide in frame with said first splice donor/acceptor pair; and
(d) nucleic acid encoding a third reporter polypeptide in frame with said second splice donor/acceptor pair;
wherein said first reporter polypeptide, said second reporter polypeptide, and said third reporter polypeptide are each a different reporter polypeptide.

2. The nucleic acid construct of claim 1, wherein said nucleic acid construct is a plasmid.

3. The nucleic acid construct of claim 1, wherein said promoter is selected from the group consisting of a CAG promoter, a tTA promoter, a CaMKII promoter, and a Syn1 promoter.

4-5. (canceled)

6. The nucleic acid construct of claim 1, wherein said first reporter polypeptide, said second reporter polypeptide, and said third reporter polypeptide are each a fluorescent polypeptide.

7. The nucleic acid construct of claim 1, wherein said first reporter polypeptide, said second reporter polypeptide, and said third reporter polypeptide are each independently selected from the group consisting of a blue fluorescent polypeptide, a green fluorescent polypeptide, a mCherry polypeptide, a emiRFP670 polypeptide, a firefly luciferase polypeptide, and a Renilla luciferase polypeptide.

8. The nucleic acid construct of claim 1, wherein said SYNGAP1 minigene comprises exons 17 and 18, and the intervening intron.

9. The nucleic acid construct of claim 8, wherein said SYNGAP1 minigene comprises a sequence set forth in SEQ ID NO:1.

10. The nucleic acid construct of claim 1, wherein said SYNGAP1 minigene comprises exons 10 and 11, and the intervening intron.

11. The nucleic acid construct of claim 10, wherein said SYNGAP1 minigene comprises a sequence set forth in SEQ ID NO:2.

12. The nucleic acid construct of claim 1, wherein said SYNGAP1 minigene comprises exons 18, 19, and 20, and the intervening introns.

13. The nucleic acid construct of claim 12, wherein said SYNGAP1 minigene comprises a sequence set forth in SEQ ID NO:3.

14. (canceled)

15. A method for identifying a splice-switching anti-sense oligonucleotide (SSO) that can modulate SYNGAP1 gene splicing, said method comprising:
(a) delivering a candidate SSO to a cell;
(b) delivering the nucleic acid construct of claim 1 to said cell; and
(c) detecting said first reporter polypeptide, said second reporter polypeptide, and said third reporter polypeptide,
wherein a SSO that modulates SYNGAP1 splicing at said first splice donor/acceptor pair is identified when said first reporter polypeptide and said second reporter polypeptide are detected; and
wherein a SSO that modulates SYNGAP1 splicing at said second splice donor/acceptor pair is identified when said first reporter polypeptide and said third reporter polypeptide are detected.

16. A method for modulating SYNGAP1 gene splicing in a cell, said method comprising:
administering to said cell an SSO that can reduce or eliminate expression of a SynGAP1-α2 polypeptide, a SynGAP1-α3 polypeptide, a SynGAP1-β polypeptide, or a SynGAP1-γ polypeptide, wherein said SSO targets a splice site within a SYNGAP1 gene.

17. A method for increasing a level of a SynGAP1-α1 polypeptide or a SynGAP1-α2 polypeptide in a cell, said method comprising, administering to said cell an SSO that targets a splice site within a SYNGAP1 gene.

18. The method of claim 16, wherein said splice site is selected from the group consisting of a splice donor site, a splice acceptor site, an exonic splice enhancer, an exonic splice silencer, an intronic splice enhancer, and an intronic splice silencer.

19. The method of claim 16, wherein said splice site comprises the nucleic acid sequence GCTCAGgtggaa (SEQ ID NO:5).

20. The method of claim 16, wherein said splice site comprises the nucleic acid sequence ttgcagGAGAGG (SEQ ID NO:7).

21. The method of claim 16, wherein said SSO comprises a nucleic acid sequence set forth in any one of SEQ ID NOs: 8-39 or SEQ ID NOs: 71-114.

22. A method for treating a mammal having or at risk of developing a SYNGAP1-associated neurodevelopmental disorder (NDD), wherein said method comprises:
administering to said mammal an SSO that targets a splice site within a SYNGAP1 gene.

23. The method of claim 22, wherein said mammal is a human.

24-25. (canceled)

26. The method of claim 22, wherein said SYNGAP1-associated NDD is selected from the group consisting of a SYNGAP1-related intellectual disability (SRID), SYNGAP1-related autism spectrum disorder (ASD), SYNGAP1-related epilepsy, sleep disorders, intellectual disability, and schizophrenia.

27. The method of claim 22, wherein said administering comprises an intracerebroventricular (ICV) injection, an intracerebral injection, a retroorbital injection, an intravenous injection, a sinus injection, or an intrathecal injection.

28-36. (canceled)
